(19) United States
(12) Patent Application Publication
(10) Pub. No.: US 2011/0034691 A1
(43) Pub. Date: Feb. 10, 2011

(54) Process for the Production of Fused, Tricyclic Sulfonamides

(75) Inventors: Michael S. Dappen, Emerald Hills, CA (US); Jacek J. Jagodzinski, Redwood City, CA (US); Lee H. Latimer, Oakland, CA (US); Jing Wu, Redwood City, CA (US); Jollie Duaine Godfrey, JR., Ewing, NJ (US); Robert Michael Matunas, Cranbury, NJ (US); Thomas W. Drabb, Trenton, NJ (US); Gary Probst, San Francisco, CA (US)

Correspondence Address: FOLEY & LARDNER LLP, 975 PAGE MILL ROAD, PALO ALTO, CA, 94304, US

(73) Assignee: Elan Pharmaceuticals, Inc.

(21) Appl. No.: 12/731,053

(22) Filed: Mar. 24, 2010

Related U.S. Application Data
(60) Provisional application No. 61/163,309, filed on Mar. 25, 2009.
(60) Provisional application No. 61/163,333, filed on Mar. 25, 2009.

(51) Int. Cl. C07D 471/04; C07D 231/12; C07D 401/12; C07D 231/16

(52) U.S. Cl. 546/82; 548/375.1; 546/275.4

(57) ABSTRACT

The present invention provides methods, i.e., scalable or large-scale processes for the production of fused, tricyclic sulfonamido analogs, such as substituted or unsubstituted 5-(aryl-sulfonyl)-4,5-dihydro-1H-pyrazolo[4,3-c]quinolines and 5-(heteroaryl-sulfonyl)-4,5-dihydro-1H-pyrazolo[4,3-c]quinolines. Exemplary methods of the invention include an intra-molecular cyclization step, in which a carbon-nitrogen bond is formed, and which employs a copper-based catalyst that contains at least one organic ligand, such as DMEDA. The invention further provides compounds, which are useful as intermediates in the methods of the invention.

### CROSS-REFERENCE TO RELATED APPLICATIONS

This application claims the benefit under 35 U.S.C. §119(e) of U.S. Provisional Patent Application No. 61/163,309 filed on Mar. 25, 2009 and from U.S. Provisional Patent Application No. 61/163,333 filed on Mar. 25, 2009, each of which is incorporated herein by reference in its entirety.

### BACKGROUND OF THE INVENTION

The invention relates to processes for the synthesis of fused, tricyclic sulfonamido analogs, such as optionally substituted 5-(aryl-sulfonyl)-4,5-dihydro-1H-pyrazolo[4,3-c]quinolines or 5-(heteroaryl-sulfonyl)-4,5-dihydro-1H-pyrazolo[4,3-c]quinolines. The invention further relates to compounds, which are useful as intermediates in the above processes, as well as methods of making such intermediates.

Certain fused, tricyclic sulfonamides inhibit gamma secretase, β-amyloid peptide release and/or β-amyloid peptide synthesis (see, e.g., U.S. Patent Application Publication 2008/0021056, incorporated herein by reference in its entirety). Such compounds are implicated in the treatment and prevention of cognitive disorders, such as Alzheimer's disease. Cost-effective processes that are amenable for large-scale production of these molecules are needed.

### SUMMARY OF THE INVENTION

This disclosure provides industrially applicable processes for obtaining the subject tricyclic sulfonamides in good yield and purity. In particular, the current disclosure provides a method of affecting an intra-molecular cyclization, the method comprising:
(i) contacting a first molecule having a structure according to Formula (I):

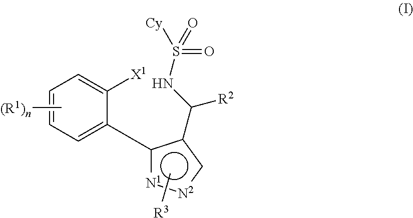

or a salt or solvate thereof,
wherein
n is an integer selected from 0 to 4;
N¹ and N² are nitrogen atoms of a pyrazole ring;
X¹ is F, Cl, Br, I, tosylate or mesylate;
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
   wherein each of the alkyl, alkenyl, alkynyl, heteroalkyl, cycloalkyl, heterocycloalkyl, aryl and heteroaryl is optionally substituted with from 1 to 3 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
   R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
   R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
R² is a member selected from H, alkyl, alkenyl, alkynyl, haloalkyl, cycloalkyl, heterocycloalkyl, aryl, heteroaryl, wherein each of the alkyl, alkenyl, alkynyl, haloalkyl, cycloalkyl, heterocycloalkyl, aryl, and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶;
R³ is an amino protecting group covalently bonded to N¹ or N² of the pyrazole; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
   each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
   each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
with a catalyst comprising copper and at least one organic ligand, under reaction conditions sufficient to form a second molecule having a structure according to Formula (II):

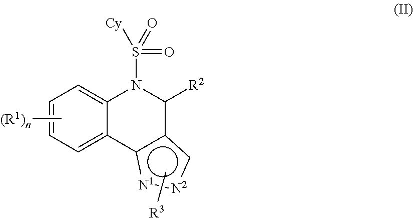

or a salt or solvate thereof, wherein Cy, n, R¹, R² and R³ are defined as for Formula (I).

In another embodiment is provided a method comprising:
(i) contacting a first compound having a structure according to Formula (X):

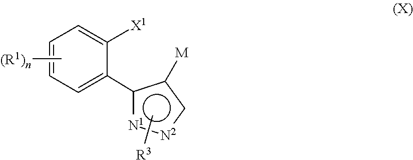

wherein
M is selected from Li and MgX, wherein X is halogen;
n is an integer selected from 0 to 4;
N¹ and N² are nitrogen atoms of a pyrazole ring;
X¹ is F, Cl, Br, I, tosylate or mesylate;
R³ is an amino protecting group covalently bonded to N¹ or N²; and
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein
   each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
   R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
   R⁶ is selected from acyl, C₁-C₆-alkyl, Cᵢ-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
      wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
   with a sulfinylimine having a structure according to Formula (XI):

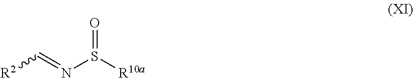

wherein
R² is selected from H, alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶; and
R^{10a} is selected from alkyl, alkenyl, alkynyl, haloalkyl, cycloalkyl, heterocycloalkyl, aryl and heteroaryl, each optionally substituted with from 1 to 5 substituents selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
   wherein
      thereby forming a second compound having a structure according to Formula (XII):

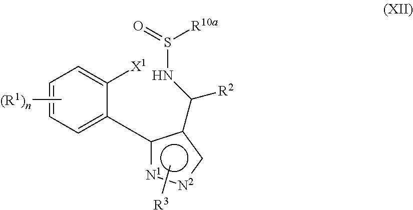

or a salt or solvate thereof.

In yet another embodiment is provided a method comprising:
(i) contacting a first compound having a structure according to Formula (Xm):

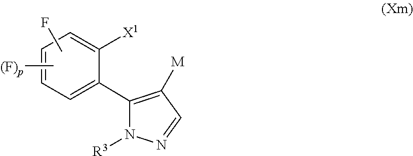

or a salt or solvate thereof,
wherein

M is Li or MgX, wherein X is Cl, Br or I;

X¹ is F, Cl or Br;

p is 0 or 1; and

R³ is an amino protecting group,

with a sulfinylimine having a structure according to Formula (XIa):

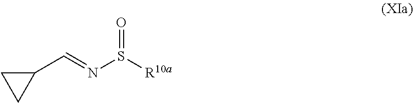

wherein R^{10a} is branched (C₃-C₈)alkyl, branched 3- to 8-membered heteroalkyl, (C₃-C₁₀)cycloalkyl, 3- to 6-membered heterocycloalkyl, aryl, and 5- or 6-membered heteroaryl,
under reaction conditions sufficient to form a second compound having a structure according to Formula (XIIa):

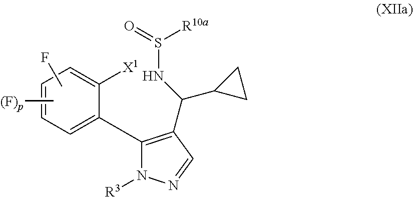

or a salt or solvate thereof; and
(ii) removing a sulfinyl moiety from the second compound of Formula (XIIa), thereby forming a third compound having a structure according to Formula (XIIIa):

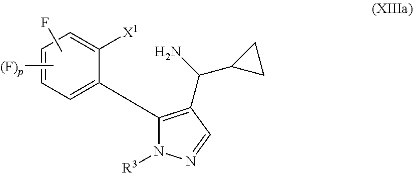

or a salt or solvate thereof.

In still yet another embodiment, is provided a method of affecting an intra-molecular cyclization, the method comprising:
(i) contacting a first molecule having a structure according to Formula (III):

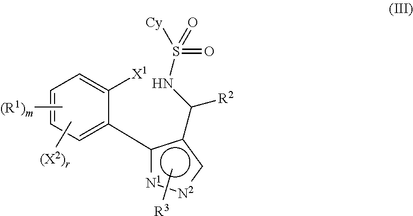

or a salt or solvate thereof,
wherein
r is an integer selected from 2 to 4;
m is an integer selected from 0 to 2, provided that the sum of m and r is not greater than 4;
N¹ and N² are nitrogen atoms of a pyrazole ring;
X¹ is F, Cl, Br, I, tosylate or mesylate;
X² is F, Cl or Br;
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
   wherein each of the alkyl, alkenyl, alkynyl, heteroalkyl, cycloalkyl, heterocycloalkyl, aryl and heteroaryl is optionally substituted with from 1 to 3 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
   R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
   R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
R² is a member selected from H, alkyl, alkenyl, alkynyl, haloalkyl, cycloalkyl, heterocycloalkyl, aryl, heteroaryl, wherein each of the alkyl, alkenyl, alkynyl, haloalkyl, cycloalkyl, heterocycloalkyl, aryl, and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶;
R³ is an amino protecting group covalently bonded to N¹ or N² of the pyrazole; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
   each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
   each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
with a base, in the absence of a metal catalyst, under reaction conditions sufficient to form a second molecule having a structure according to Formula (IV):

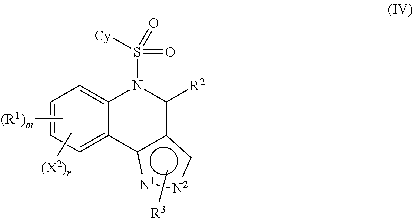

or a salt or solvate thereof, wherein Cy, m, r, X², R¹, R² and R³ are defined as for Formula (I).

This invention is also directed to useful intermediates in the methods just described. In one embodiment, the invention is directed to a compound having a structure according to Formula (XX):

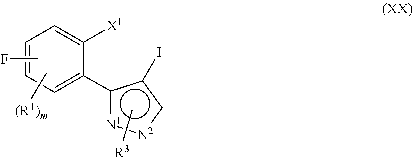

or a salt or solvate thereof,
wherein
N¹ and N² are nitrogen atoms of a pyrazole ring;
I is iodine;
X¹ is halogen;
R³ is an amino protecting group covalently bonded to N¹ or N² of the pyrazole ring;
m is an integer selected from 0 to 3; and
each R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
   wherein each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
   wherein
      R¹⁴, R¹⁵, and R¹⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
      R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
   R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
   R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.

In another embodiment is provided a compound having a structure according to Formula (XXII):

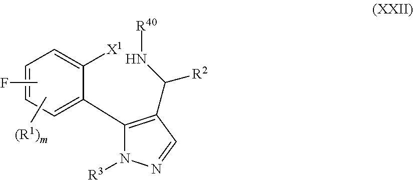

or a salt or solvate thereof,
wherein
X¹ is halogen;
R³ is an amino protecting group;
m is an integer selected from 0 to 3;
each R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
   wherein each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR14R¹⁵, S(O)R¹⁶ and S(O)₂R¹⁶,
   R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
   R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.
R² is selected from H, alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶, with the proviso that R² is other than carboxyl or carboxyl-substituted C₁-C₃-alkyl;
R⁴⁰ is selected from H, alkyl, alkenyl, alkynyl, haloalkyl, cycloalkyl, heterocycloalkyl, aryl, heteroaryl, S(O)R^{10a}, and S(O)₂Cy,
wherein
   each of the alkyl, alkenyl, alkynyl, haloalkyl, cycloalkyl, heterocycloalkyl, aryl, and heteroaryl of R⁴⁰ is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
R^{10a} is selected from alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
   wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.

In yet another embodiment is provided a compound selected from:
5-(2-bromo-5-fluorophenyl)-1-tert-butyl-4-iodo-1H-pyrazole;
5-(2-bromo-4-fluorophenyl)-1-tert-butyl-4-iodo-1H-pyrazole;
5-(2-bromo-4,5-difluorophenyl)-1-tert-butyl-4-iodo-1H-pyrazole; and
1-tert-butyl-4-iodo-5-(2,4,5-trifluorophenyl)-1H-pyrazole;
(5-(2-bromo-5-fluorophenyl)-1-tert-butyl-1H-pyrazol-4-yl)(cyclopropyl)methanamine;
(5-(2-bromo-4-fluorophenyl)-1-tert-butyl-1H-pyrazol-4-yl)(cyclopropyl)methanamine;
(5-(2-bromo-4,5-difluorophenyl)-1-tert-butyl-1H-pyrazol-4-yl)(cyclopropyl)methanamine;
(1-tert-butyl-5-(2,4,5-trifluorophenyl)-1H-pyrazol-4-yl)(cyclopropyl)methanamine;
(1R)-(5-(2-bromo-5-fluorophenyl)-1-tert-butyl-1H-pyrazol-4-yl)(cyclopropyl)methanamine;
(1R)-(5-(2-bromo-4-fluorophenyl)-1-tert-butyl-1H-pyrazol-4-yl)(cyclopropyl)methanamine;
(1R)-(5-(2-bromo-4,5-difluorophenyl)-1-tert-butyl-1H-pyrazol-4-yl)(cyclopropyl)methanamine; and
(1R)-(1-tert-butyl-5-(2,4,5-trifluorophenyl)-1H-pyrazol-4-yl)(cyclopropyl)methanamine;
N-((5-(2-bromo-5-fluorophenyl)-1-tert-butyl-1H-pyrazol-4-yl)(cyclopropyl)methyl)-2-methylpropane-2-sulfinamide;
N-((1R)-(5-(2-bromo-5-fluorophenyl)-1-tert-butyl-1H-pyrazol-4-yl)(cyclopropyl)methyl)-2-methylpropane-2-sulfinamide;
N-((5-(2-bromo-4,5-difluorophenyl)-1-tert-butyl-1H-pyrazol-4-yl)(cyclopropyl)methyl)-2-methylpropane-2-sulfinamide;
N-((1R)-(5-(2-bromo-4,5-difluorophenyl)-1-tert-butyl-1H-pyrazol-4-yl)(cyclopropyl)methyl)-2-methylpropane-2-sulfinamide;
N-((5-(2-bromo-4-fluorophenyl)-1-tert-butyl-1H-pyrazol-4-yl)(cyclopropyl)methyl)-2-methylpropane-2-sulfinamide;
N-((1R)-(5-(2-bromo-4-fluorophenyl)-1-tert-butyl-1H-pyrazol-4-yl)(cyclopropyl)methyl)-2-methylpropane-2-sulfinamide;
N-((1-tert-butyl-5-(2,4,5-trifluorophenyl)-1H-pyrazol-4-yl)(cyclopropyl)methyl)-2-methylpropane-2-sulfinamide; and
N-((1R)-(1-tert-butyl-5-(2,4,5-trifluorophenyl)-1H-pyrazol-4-yl)(cyclopropyl)methyl)-2-methylpropane-2-sulfinamide;
N-((5-(2-bromo-4-fluorophenyl)-1-tert-butyl-1H-pyrazol-4-yl)(cyclopropyl)methyl)-4-(trifluoromethyl)benzenesulfonamide;
N-((1R)-(5-(2-bromo-4-fluorophenyl)-1-tert-butyl-1H-pyrazol-4-yl)(cyclopropyl)methyl)-4-(trifluoromethyl)benzenesulfonamide;
N-((5-(2-bromo-4-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4-fluorophenyl)-1-tert-butyl-1H-pyrazol-4-yl)(cyclopropyl)methyl)-6-(trifluoromethyl)pyridine-3-sulfonamide;
N-((5-(2-bromo-4,5-difluorophenyl)-1-tert-butyl-1H-pyrazol-4-yl)(cyclopropyl)methyl)-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-4-(trifluoromethyl)benzenesulfonamide;
N-((5-(2-bromo-4,5-difluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4,5-difluorophenyl)-1-tert-butyl-1H-pyrazol-4-yl)(cyclopropyl)methyl)-6-(trifluoromethyl)pyridine-3-sulfonamide;
N-((5-(2-bromo-5-fluorophenyl)-1-tert-butyl-1H-pyrazol-4-yl)(cyclopropyl)methyl)-4-(trifluoromethyl)benzenesulfonamide;
N-((1R)-(5-(2-bromo-5-fluorophenyl)-1-tert-butyl-1H-pyrazol-4-yl)(cyclopropyl)methyl)-4-(trifluoromethyl)benzenesulfonamide;
N-((5-(2-bromo-5-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide;
N-((1-tert-butyl-5-(2,4,5-trifluorophenyl)-1H-pyrazol-4-yl)(cyclopropyl)methyl)-4-(trifluoromethyl)benzenesulfonamide;
N-((1R)-(1-tert-butyl-5-(2,4,5-trifluorophenyl)-1H-pyrazol-4-yl)(cyclopropyl)methyl)-4-(trifluoromethyl)benzenesulfonamide;
N-((1-tert-butyl-5-(2,4,5-trifluorophenyl)-1H-pyrazol-4-yl)(cyclopropyl)methyl)-6-(trifluoromethyl)pyridine-3-sulfonamide;
N-((1R)-(1-tert-butyl-5-(2,4,5-trifluorophenyl)-1H-pyrazol-4-yl)(cyclopropyl)methyl)-6-(trifluoromethyl)pyridine-3-sulfonamide,
N-((1R)-(1-tert-butyl-5-(2,4,5-trifluorophenyl)-1H-pyrazol-4-yl)(cyclopropyl)methyl)-3-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-5-fluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-3-methoxy-4-(trifluoromethyl)benzenesulfonamide;
N-((1R)-(1-tert-butyl-5-(2,4,5-trifluorophenyl)-1H-pyrazol-4-yl)(cyclopropyl)methyl)-2-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-2-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-2-methoxy-4-(trifluoromethyl)benzenesulfonamide; and
N-((1R)-(5-(2-bromo-5-fluorophenyl)-1-tert-butyl-1H-pyrazol-4-yl)(cyclopropyl)methyl)-2-methoxy-4-(trifluoromethyl)benzenesulfonamide,
or a salt, solvate, tautomer or mixture of tautomers thereof.

Additional embodiments of the invention are found throughout the disclosure and in the claims.

### BRIEF DESCRIPTION OF THE DRAWINGS

FIG. 1 is a scheme illustrating an exemplary intra-molecular cyclization as described in this disclosure. In FIG. 1, N¹ and N² are nitrogen atoms of a pyrazole ring; n is an integer selected from 0 to 4; X¹ is a leaving group (e.g., Br); R³ is an amino protecting group as defined herein (e.g., tert-butyl); and R¹, R², and Cy are as defined in the specification, e.g., for Formula (I) and Formula (II). In one example in FIG. 1, the amino protecting group R³ is covalently bonded to N¹ of the pyrazole ring.

FIG. 2 is a scheme illustrating exemplary methods of this disclosure. In FIG. 2, n is an integer selected from 0 to 4; M is MgX or Li, wherein X is Cl, Br or I; X¹ is F, Cl or Br; Cy, R¹, R² and R³ are as defined in the specification, e.g., for Formula (I) and Formula (II). In one example in FIG. 2, the amino protecting group R³ is tert-butyl. In another example in FIG. 2, X¹ is Br.

FIG. 3 is a scheme illustrating exemplary methods of this disclosure. In FIG. 3, p is an integer selected from 0 and 1; M is MgX or Li, wherein X is Cl, Br or I; E is N or CH; and R³ and R¹⁰ are as defined in the specification, e.g., for Formula (I) and Formula (Ic), respectively. In one example in FIG. 3, R¹⁰ is CF₃. In another example in FIG. 3, p is 1 and E is CH. In yet another example in FIG. 3, p is 0 and E is N.

FIG. 4 is a scheme illustrating exemplary methods of this disclosure. In FIG. 4, n is an integer selected from 0 to 4; X¹ is F, Cl or Br; X is Cl, Br or I; and R¹ and R³ are as defined in the specification, e.g., for Formula (I), and Formula (II). In one example in FIG. 4, the amino protecting group R³ is tert-butyl. In one example in FIG. 4, X¹ is Br. In another example in FIG. 4, X¹ is F.

FIG. 5 is a scheme illustrating exemplary methods of this disclosure. In FIG. 5, p is an integer selected from 1 and 0; X is Cl, Br or I; and X¹ is F, Cl or Br.

FIG. 6 is a scheme illustrating exemplary methods of this disclosure. In FIG. 6, m is an integer selected from 0 to 3; r is an integer selected from 1 to 4 (e.g., 2 to 4), with the proviso that the sum of m and r is not greater than 4; M is MgX or Li, wherein X is Cl, Br or I; and X², Cy, R¹, R² and R³ are as defined in the specification, e.g., for Formula (I), Formula (II) and Formula (III), respectively. In one example in FIG. 6, the amino protecting group R³ is tert-butyl. In another example in FIG. 6, X² is halogen (e.g., F, Cl or Br). In yet another example in FIG. 6, X¹ is F.

FIG. 7 is a scheme illustrating exemplary methods of this disclosure. In FIG. 7, p is an integer selected from 0 and 1; M is MgX or Li, wherein X is Cl, Br or I; E is N or CH; and R³ and R¹⁰ are as defined in the specification, e.g., for Formula (I) and Formula (Ic), respectively. In one example in FIG. 7, R¹⁰ is CF₃. In another example in FIG. 7, p is 1 and E is CH. In yet another example in FIG. 7, p is 0 and E is N.

### DETAILED DESCRIPTION OF THE INVENTION

### I. Definitions

The definitions and explanations below are for the terms as used throughout this entire document including both the specification and the claims. Throughout the specification and the appended claims, a given formula or name shall encompass all isomers thereof, such as stereoisomers, geometrical isomers, optical isomers, tautomers, and mixtures thereof where such isomers exist, as well as pharmaceutically acceptable salts and solvates thereof, such as hydrates.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5% or 1%.

Where multiple substituents are indicated as being attached to a structure, those substituents are independently chosen. For example “ring A is optionally substituted with 1, 2 or 3 R_{q} groups” indicates that ring A is substituted with 1, 2 or 3 R_{q} groups, wherein the R_{q} groups are independently chosen (i.e., can be the same or different).

Compounds were named using Autonom 2000 4.01.305, which is available from Beilstein Information Systems, Inc, Englewood, Colo.; ChemDraw v. 10.0, (available from Cambridgesoft at 100 Cambridge Park Drive, Cambridge, Mass. 02140), or ACD Name pro, which is available from Advanced Chemistry Development, Inc., at 110 Yonge Street, 14^{th} floor, Toronto, Ontario, Canada M5c 1T4. Alternatively, the names were generated based on the IUPAC rules or were derived from names originally generated using the aforementioned nomenclature programs. A person of skill in the art will appreciate that chemical names for tautomeric forms of the current compounds will vary slightly, but will nevertheless describe the same compound. For example, the names 4-cyclopropyl-7,8-difluoro-5-(4-(trifluoromethyl)phenylsulfonyl)-4,5-dihydro-1H-pyrazolo[4,3-c]quinoline and 4-cyclopropyl-7,8-difluoro-5-(4-(trifluoromethyl)-phenylsulfonyl)-4,5-dihydro-2H-pyrazolo[4,3-c]quinoline describe two tautomeric forms of the same compound.

Where substituent groups are specified by their conventional chemical formulae, written from left to right, they equally encompass the chemically identical substituents, which would result from writing the structure from right to left. For example, “—CH₂O—” is intended to also recite “—OCH₂—”.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one embodiment, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁₀, or C₂-C₈) and at least one heteroatom chosen , e.g., from N, O, S, Si, B and P (in one embodiment, N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl”, “heterocyclic”, “heterocycle”, or “heterocyclyl”, by itself or in combination with other terms, represents a carbocyclic, non-aromatic ring (e.g., 3- to 8-membered ring and for example, 4-, 5-, 6- or 7-membered ring) containing at least one and up to 5 heteroatoms selected from, e.g., N, O, S, Si, B and P (for example, N, O and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule. Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. In one embodiment, the aryl group is selected from phenyl, benzo[d][1,3]dioxolyl and naphthyl. The aryl group, in yet another embodiment, is phenyl.

The term “arylalkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” groups include benzyl, phenethyl, pyridylmethyl and the like.

By “aryloxy” is meant the group —O-aryl, where aryl is as defined herein. In one example, the aryl portion of the aryloxy group is phenyl or naphthyl. The aryl portion of the aryloxy group, in one embodiment, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si and B (for example, N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom. In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable aryl group substituents described below.

For brevity, the term “aryl” when used in combination with other terms (e.g., aryloxy, arylthioxy, arylalkyl) includes both aryl and heteroaryl rings as defined above.

Each of the above terms (e.g., “alkyl”, “cycloalkyl”, “heteroalkyl”, heterocycloalkyl“, “aryl” and “heteroaryl”) are meant to include both substituted and unsubstituted forms of the indicated radical. The term “substituted” for each type of radical is explained below. When a compound of the present disclosure includes more than one substituent, then each of the substituents is independently chosen.

The term “substituted” in connection with alkyl, alkenyl, alkynyl, cycloalkyl, heteroalkyl and heterocycloalkyl radicals (including those groups referred to as alkylene, heteroalkylene, heteroalkenyl, cycloalkenyl, heterocycloalkenyl, and the like) refers to one or more substituents, wherein each substituent is independently selected from, but not limited to, 3- to 10-membered heteroalkyl, C₃-C₁₀ cycloalkyl, 3- to 10-membered heterocycloalkyl, aryl, heteroaryl, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{e}, —S(O)₂R^{e}, —S(O)₂NR^{a}R^{b}, —NR^{c}S(O)₂R^{a}, —CN and —NO₂. R^{a}, R^{b}, R^{c}, R^{d} and R^{e} each independently refer to hydrogen, C₁-C₂₄ alkyl (e.g., C₁-C₁₀ alkyl or C₁-C₆ alkyl), C₃-C₁₀ cycloalkyl, C₁-C₂₄ heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl, wherein, in one embodiment, R^{e} is not hydrogen. When two of the above R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N-alkyl-piperidinyl and morpholinyl.

The term “substituted” in connection with aryl and heteroaryl groups, refers to one or more substituents, wherein each substituent is independently selected from, but not limited to, alkyl (e.g., C₁-C₂₄ alkyl, C₁-C₁₀ alkyl or C₁-C₆ alkyl), cycloalkyl (e.g., C₃-C₁₀ cycloalkyl, or C₃-C₈ cycloalkyl), alkenyl (e.g., C₁-C₁₀ alkenyl or C₁-C₆ alkenyl), alkynyl (e.g., C₁-C₁₀ alkynyl or C₁-C₆ alkynyl), heteroalkyl (e.g., 3- to 10-membered heteroalkyl), heterocycloalkyl (e.g., C₃-C₈ heterocycloalkyl), aryl, heteroaryl, —R^{a}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{e}, —S(O)₂R^{e}, —S(O)₂NR^{a}R^{b}, —NR^{c}S(O)₂R^{a}, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system, wherein R^{a}, R^{b}, R^{c}, R^{d} and R^{e} each independently refer to hydrogen, C₁-C₂₄ alkyl (e.g., C₁-C₁₀ alkyl or C₁-C₆ alkyl), C₃-C₁₀ cycloalkyl, C₁-C₂₄ heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl, wherein, in one embodiment, R^{e} is not hydrogen. When two R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N-alkyl-piperidinyl and morpholinyl.

The term “substituted” in connection with aryl and heteroaryl groups also refers to one or more fused ring(s), in which two hydrogen atoms on adjacent atoms of the aryl or heteroaryl ring are optionally replaced with a substituent of the formula -T-C(O)—(CRR′)_{q}—U—, wherein T and U are independently —NR—, —O—, —CRR′— or a single bond, and q is an integer from 0 to 3. Alternatively, two of the hydrogen atoms on adjacent atoms of the aryl or heteroaryl ring can optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer from 1 to 4. One of the single bonds of the ring so formed can optionally be replaced with a double bond. Alternatively, two of the hydrogen atoms on adjacent atoms of the aryl or heteroaryl ring can optionally be replaced with a substituent of the formula —(CRR′)ₛ—X—(CR″R′″)_{d}—, where s and d are independently integers from 0 to 3, and X is —O—, —NR′—, —S—, —S(O)—, —S(O)₂—, or —S(O)₂NR′—, wherein the substituents R, R′, R″ and R′″ in each of the formulas above are independently selected from hydrogen and (C₁-C₆)alkyl.

The terms “halo” or “halogen,” by themselves or as part of another substituent, mean at least one of fluorine, chlorine, bromine and iodine.

By “haloalkyl” is meant an alkyl radical, wherein alkyl is as defined above and wherein at least one hydrogen atom is replaced by a halogen atom. The term “haloalkyl,” is meant to include monohaloalkyl and polyhaloalkyl. For example, the term “halo(C₁-C₄)alkyl” or “(C₁-C₄)haloalkyl” is mean to include, but not limited to, chloromethyl, 1-bromoethyl, fluoromethyl, difluoromethyl, trifluoromethyl, 1,1,1-trifluoroethyl and 4-chlorobutyl, 3-bromopropyl.

As used herein, the term “acyl” describes the group —C(O)R^{e}, wherein R^{e} is selected from hydrogen, C₁-C₂₄ alkyl (e.g., C₁-C₁₀ alkyl or C₁-C₆ alkyl), C₁-C₂₄ alkenyl (e.g., C₁-C₁₀ alkenyl or C₁-C₆ alkenyl), C₁-C₂₄ alkynyl (e.g., C₁-C₁₀ alkynyl or C₁-C₆ alkynyl), C₃-C₁₀ cycloalkyl, C₁-C₂₄ heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl. In one embodiment, R^{e} is not hydrogen.

By “alkanoyl” is meant an acyl radical —C(O)-Alk-, wherein Alk is an alkyl radical as defined herein. Examples of alkanoyl include acetyl, propionyl, butyryl, isobutyryl, valeryl, isovaleryl, 2-methyl-butyryl, 2,2-dimethylpropionyl, hexanoyl, heptanoyl, octanoyl and the like.

As used herein, the term “heteroatom” includes oxygen (O), nitrogen (N), sulfur (S), silicon (Si), boron (B) and phosphorus (P). In one embodiment, heteroatoms are O, S and N.

By “oxo” is meant the group ═O.

By “sulfonyl” or “sulfonyl group” is meant a group that is connected to the remainder of a molecule via a —S(O)₂— moiety. Hence sulfonyl can be —S(O)₂R, wherein R is, e.g., NHR′, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. An exemplary sulfonyl group is S(O)₂-Cy, wherein Cy is, e.g., substituted or unsubstituted aryl or substituted or unsubstituted heteroaryl.

By “sulfinyl” or “sulfinyl group” is meant a group that is connected to the remainder of the molecule via a —S(O)— moiety. Hence, sulfinyl can be —S(O)R, wherein R is as defined for sulfonyl group.

By “sulfonamide” is meant a group having the formula —S(O)₂NRR, where each of the R variables are independently selected from the variables listed above for R.

The symbol “R” is a general abbreviation that represents a substituent group as described herein. Exemplary substituent groups include alkyl, alkenyl, alkynyl, cycloalkyl, heteroalkyl, aryl, heteroaryl and heterocycloalkyl groups, each as defined herein.

As used herein, the term “aromatic ring” or “non-aromatic ring” is consistent with the definition commonly used in the art. For example, aromatic rings include phenyl and pyridyl. Non-aromatic rings include cyclohexanes.

As used herein, the term “fused ring system” means at least two rings, wherein each ring has at least 2 atoms in common with another ring. “Fused ring systems can include aromatic as well as non-aromatic rings. Examples of “fused ring systems” are naphthalenes, indoles, quinolines, chromenes and the like. Likewise, the term “fused ring” refers to a ring that has at least two atoms in common with the ring to which it is fused.

The term compound and molecule are used interchangeably. Other forms contemplated by the invention when the word “molecule” or “compound” is employed are salts, prodrugs, solvates, tautomers, stereoisomers and mixtures of stereoisomers. In some embodiments, the salts are pharmaceutically acceptable salts.

The term “pharmaceutically acceptable” refers to those properties and/or substances that are acceptable to a patient (e.g., human patient) from a toxicological and/or safety point of view.

The term “pharmaceutically acceptable salts” means salts of the compounds of the present disclosure, which may be prepared with relatively nontoxic acids or bases, depending on the particular substituents found on the compounds described herein. When compounds of the present disclosure contain relatively acidic functionalities (e.g., —COOH group), base addition salts can be obtained by contacting the compound (e.g., neutral form of such compound) with a sufficient amount of the desired base, either neat or in a suitable inert solvent. Examples of pharmaceutically acceptable base addition salts include lithium, sodium, potassium, calcium, ammonium, organic amino, magnesium and aluminum salts and the like. When compounds of the present disclosure contain relatively basic functionalities (e.g., amines), acid addition salts can be obtained, e.g., by contacting the compound (e.g., neutral form of such compound) with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, diphosphoric, monohydrogenphosphoric, dihydrogenphosphoric, sulfuric, monohydrogensulfuric, hydriodic and the like, as well as the salts derived from relatively nontoxic organic acids like formic, acetic, propionic, isobutyric, malic, maleic, malonic, benzoic, succinic, suberic, fumaric, lactic, mandelic, phthalic, benzenesulfonic, p-tolylsulfonic, citric, tartaric, methanesulfonic, 2-hydroxyethylsulfonic, salicylic, stearic and the like. Also included are salts of amino acids such as arginate and the like, and salts of organic acids like glucuronic or galactunoric acids and the like (see, for example, Berge et al., Journal of Pharmaceutical Science, 1977, 66: 1-19). Certain specific compounds of the present disclosure contain both, basic and acidic, functionalities that allow the compounds to be converted into either base or acid addition salts.

The neutral forms of the compounds can be regenerated, for example, by contacting the salt with a base or acid and isolating the parent compound in the conventional manner. The parent form of the compound can differ from the various salt forms in certain physical properties, such as solubility in polar solvents, but otherwise the salts are equivalent to the parent form of the compound for the purposes of the present disclosure.

When a substituent includes a negatively charged oxygen atom “O⁻, e.g., in “—COO⁻”, then the formula is meant to optionally include a proton or an organic or inorganic cationic counterion (e.g., Na+). In one example, the resulting salt form of the compound is pharmaceutically acceptable. Further, when a compound of the present disclosure includes an acidic group, such as a carboxylic acid group, e.g., written as the substituent “—COOH”, “—CO₂H” or “—C(O)₂H”, then the formula is meant to optionally include the corresponding “de-protonated” form of that acidic group, e.g., “—COO⁻”, “—CO₂⁻” or “—C(O)₂⁻”, respectively.

In addition to salt forms, the present disclosure provides compounds, which are in a prodrug form. Prodrugs of the compounds described herein are those compounds that readily undergo chemical changes under physiological conditions to provide the compounds of the present disclosure. Non-limiting examples of “pharmaceutically acceptable derivative” or “prodrug” include pharmaceutically acceptable esters, phosphate esters or sulfonate esters thereof as well as other derivatives of a compound of this present disclosure which, upon administration to a recipient, is capable of providing, either directly or indirectly, a compound of this present disclosure. In one embodiment, derivatives or prodrugs are those that increase the bioavailability of the compounds of this present disclosure when such compounds are administered to a mammal (e.g., by allowing an orally administered compound to be more readily absorbed into the blood stream) or which enhance delivery of the parent compound to a biological compartment (e.g., the brain or lymphatic system) relative to the parent species.

Prodrugs include a variety of esters (i.e., carboxylic acid ester). Ester groups, which are suitable as prodrug groups are generally known in the art and include benzyloxy, di(C₁-C₆)alkylaminoethyloxy, acetoxymethyl, pivaloyloxymethyl, phthalidoyl, ethoxycarbonyloxyethyl, 5-methyl-2-oxo-1,3-dioxol-4-yl methyl, and (C₁-C₆)alkoxy esters, optionally substituted by N-morpholino and amide-forming groups such as di(C₁-C₆)alkylamino. For example, ester prodrug groups include C₁-C₆ alkoxy esters. Those skilled in the art will recognize various synthetic methodologies that may be employed to form pharmaceutically acceptable prodrugs of the compounds of the present disclosure (e.g., via esterification of a carboxylic acid group).

In an exemplary embodiment, the prodrug is suitable for treatment/prevention of those diseases and conditions that require the drug molecule to cross the blood brain barrier. In one embodiment, the prodrug enters the brain, where it is converted into the active form of the drug molecule. In another example, a prodrug is used to enable an active drug molecule to reach the inside of the eye after topical application of the prodrug to the eye. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure can exist in multiple crystalline or amorphous forms (“polymorphs”). In general, all physical forms are of use in the methods contemplated by the present disclosure and are intended to be within the scope of the present disclosure. “Compound or a pharmaceutically acceptable salt, hydrate, polymorph or solvate of a compound” intends the inclusive meaning of “and/or”, in that materials meeting more than one of the stated criteria are included, e.g., a material that is both a salt and a solvate is encompassed.

The compounds of the present disclosure can contain unnatural proportions of atomic isotopes at one or more of the atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure. Compounds described herein, in which one or more of the hydrogen atoms are replaced with another stable isotope of hydrogen (i.e., deuterium) or a radioactive isotope (i.e., tritium), are part of this disclosure.

The term “solvate” is intended to refer to a complex formed by combination of solute molecules or ions with solvent molecules. The solvent can be an organic compound, an inorganic compound, or a mixture of both. Exemplary solvents for the formation of solvates include, but are not limited to, methanol, N,N-dimethylformamide, tetrahydrofuran, dimethylsulfoxide, toluene, and water. In one embodiment, solvents having a higher boiling point, such as for example, DMF, DMA, and the like.

The term “tautomer” is intended to refer to alternate forms of a compound that differ in the position of a proton, such as enol keto and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

### Compositions Including Stereoisomers

Compounds of the present disclosure can exist in particular geometric or stereoisomeric forms. The present disclosure contemplates all such compounds, including cis- and trans-isomers, (−)- and (+)-enantiomers, diastereomers, (D)-isomers, (L)-isomers, the racemic mixtures thereof, and other mixtures thereof, such as enantiomerically or diastereomerically enriched mixtures, as falling within the scope of the present disclosure. Additional asymmetric carbon atoms can be present in a substituent such as an alkyl group. All such isomers, as well as mixtures thereof, are intended to be included in this disclosure. When the compounds described herein contain olefinic double bonds or other centers of geometric asymmetry, and unless specified otherwise, it is intended that the compounds include both E and Z geometric isomers. Likewise, all tautomeric forms and mixtures of tautomers are included.

Optically active (R)- and (S)-isomers and d and l isomers can be prepared using chiral synthons or chiral reagents, or resolved using conventional techniques. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound. If, for instance, a particular enantiomer of a compound of the present disclosure is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

As used herein, the term “chiral”, “enantiomerically enriched” or “diastereomerically enriched” refers to a compound having an enantiomeric excess (ee) or a diastereomeric excess (de) of greater than about 50%, for example, greater than about 70%, such as greater than about 90%. In one embodiment, the compositions have higher than about 90% enantiomeric or diastereomeric excess, e.g., those compositions with greater than about 95%, greater than about 97% and greater than about 99% ee or de.

The terms “enantiomeric excess” and “diastereomeric excess” are used in their conventional sense. Compounds with a single stereocenter are referred to as being present in “enantiomeric excess”, those with at least two stereocenters are referred to as being present in “diastereomeric excess”. The value of ee will be a number from 0 to 100, zero being racemic and 100 being enantiomerically pure. For example, a 90% ee reflects the presence of 95% of one enantiomer and 5% of the other(s) in the material in question.

Hence, in one embodiment, the disclosure provides a composition including a first stereoisomer and at least one additional stereoisomer of a compound of the present disclosure. The first stereoisomer can be present in a diastereomeric or enantiomeric excess of at least about 80%, such as at least about 90%, and for example, at least about 95%. In another embodiment, the first stereoisomer is present in a diastereomeric or enantiomeric excess of at least about 96%, at least about 97%, at least about 98%, at least about 99% or at least about 99.5%. In yet another embodiment, the compounds of the present disclosure is enantiomerically or diastereomerically pure (diastereomeric or enantiomeric excess is about 100%). Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

“Amino-protecting group” refers to those organic groups intended to protect the nitrogen atom against undesirable reactions during synthetic procedures and includes, but is not limited to, silyl ethers, such as 2-(trimethylsilyl)ethoxymethyl (SEM) ether, or alkoxymethyl ethers, such as methoxymethyl (MOM) ether, tert-butoxymethyl (BUM) ether, benzyloxymethyl (BOM) ether or methoxyethoxymethyl (MEM) ether. Additional protecting groups include, tert-butyl, acetyl, benzyl, benzyloxycarbonyl (carbobenzyloxy, CBZ), p-methoxybenzyloxycarbonyl, p-nitrobenzyloxycarbonyl, tert-butoxycarbonyl (BOC), trifluoroacetyl, and the like.

Certain protecting groups may be preferred over others due to their convenience or relative ease of removal, or due to their stereo specific effects in subsequent steps of the process. Additional suitable amino protecting groups are taught in T. W. Greene and P. G. M. Wuts, Protecting Groups in Organic Synthesis, Third Edition, Wiley, N.Y., 1999, and references cited therein which are all incorporated by reference in its entirety.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, or catalyst, etc. Reaction conditions may be named after the particular chemical reaction in which the conditions are employed, such as, coupling conditions, hydrogenation conditions, acylation conditions, reduction conditions, etc. Reaction conditions for known reactions are generally known to those skilled in the art or can be readily obtained from the literature. It is also contemplated that the reaction conditions can include reagents in addition to those listed with the specific reaction.

The term “isolated” or “isolating” in conjunction with a compound of this disclosure, refers to a compound that is essentially separated from other reactants of a reaction mixture (e.g., conventional work-up and/or is subjected to purification, e.g., crystallization or chromatography). An isolated compound is also essentially stripped of liquid solvent. In one example, the isolated compound is essentially dried (e.g., can be weight to determine reaction yield). For example, a compound is “not isolated” after a reaction or a reaction sequence, when it is used for the next reaction step essentially without purification (e.g., removal of other reactants, i.e., by conventional workup and/or chromatography or crystallization). The term “isolating” excludes solvent-swapping. For example, the compound is “not isolated”, when the crude reaction product is merely transferred into another solvent, e.g., by at least partial removal of one solvent (e.g., by distillation) and addition of another solvent.

“Deprotection”, “deprotecting”, “removal” or “removing” (or grammatical variation thereof) in connection with a protecting group, refers to the process by which a protecting group (e.g., an amino-protecting group) is removed from a molecule (e.g., after completion of a reaction or reaction sequence, which required the protecting group). Protected molecules may be deprotected by standard means as appropriate for the specific protecting group utilized as described, for example, in T. W. Greene and P. G. M. Wuts, Protecting Groups in Organic Synthesis, Third Edition, Wiley, N.Y., 1999, and references cited therein. Reagents suitable for the deprotection of protected amino groups include but are not limited to hydrogenolysis and treatment with acids. In the case of protected pyrazoles, deprotection can be affected, e.g., by common acidic, nucleophilic, oxidative or reductive conditions to yield the free NH-pyrazole. For example, removal of a tert-butyl group, which is covalently bonded to a nitrogen atom of a pyrazole ring, is accomplished, e.g., by treatment with aqueous acid, including but not limited to hydrochloric acid and formic acid.

The term “acid” is intended to refer to a chemical species that can either donate a proton or accept a pair of electrons from another species. Examples of acids include organic acids, carboxylic acids, sulfonic acids, mineral acids, Lewis acids, etc.

The term “Lewis acid” is used herein according to its generally accepted meaning in the art. For example, “Lewis acid” means a molecule or ion that can combine with another molecule or ion by forming a covalent bond with two electrons from the second molecule or ion. For use in the process of the invention, a Lewis acid is considered as an electron deficient species that can accept a pair of electrons. Examples of Lewis acids that can be used in the present invention are cations of metals and their complexes including magnesium, calcium, aluminum, zinc, titanium, chromium, copper, boron, tin, mercury, iron, manganese, cadmium, gallium and barium. Their complex may include hydroxides, alkyls, alkoxides, halides and organic acid ligands such as acetates. Preferred examples of Lewis acids useful in the instant process are titanium alkoxides, particularly Ti(OEt)₄ which additionally possesses dehydrating properties.

The term “base” is intended to refer to a chemical species that are proton acceptors. Suitable bases for use in the present invention include inorganic or organic bases. Examples of inorganic base include but are not limited to potassium hydroxide (KOH), barium hydroxide (Ba(OH)₂), caesium hydroxide (CsOH), sodium hydroxide (NaOH), strontium hydroxide (Sr(OH)₂), calcium hydroxide (Ca(OH)₂), lithium hydroxide (LiOH), rubidium hydroxide (RbOH), and magnesium hydroxide (Mg(OH)₂). Organic bases can be neutral or negatively charges compounds which typically contain nitrogen atoms such as amines and nitrogen-containing heterocyclic compounds. Examples of neutral nitrogen containing organic bases include ammonia, pyridine, methyl amine, imidazole, 2,2,6,6-tetramethylpiperidine, 4-(dimethylamino)pyridine and the like. Examples of negatively charged organic bases includes alkyl lithium reagents, lithium dialkylamides, lithium alkyloxides, alkylmagnesium halides and the like.

The term “large-scale” in connection with the methods described in this disclosure means that the method can produce (e.g., safely produce) of at least 10 g (e.g., at least 100 g or at least 1 kg) of the indicated product. A person of ordinary skill in the art will be able to determine whether or not a method is amenable for large-scale production (e.g., production of commercial quantities). For example, reaction steps which are associated with safety concerns, or require instant heating of the reaction mixture to a very high temperature (e.g., at least 100° C. or at least 150° C.) are often not suitable for large-scale production.

The term “catalyst” is intended to refer to a substance which, when used in certain chemical reactions, usually used in small amounts relative to the reactants, that modifies and increases the rate of a reaction without being consumed in the process. Catalysts can be heterogeneous or homogeneous, organic or transition metal-based. Catalysts useful in this invention are discussed below.

The following numbering scheme is used when numbering ring positions of the tricyclic core structure shown below:

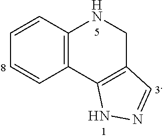

### II. Methods

Gamma secretase inhibitors are useful in the treatment and prevention of cognitive disorders, such as Alzheimer's disease. Fused, tricyclic sulfonamides are known to inhibit gamma secretase, β-amyloid peptide release and/or β-amyloid peptide synthesis and have previously been synthesized (see, e.g., U.S. Patent Application Publication 2008/0021056, incorporated herein by reference in its entirety). However, safe and cost-effective processes, which are amenable for the large-scale (e.g., at least 10 g or at least 100 g) production of these molecules have not been described.

The current disclosure describes improved processes (i.e., large-scale processes) for the production of 5-(sulfonyl)-4,5-dihydro-1H-pyrazolo[4,3-c]quinolines, such as substituted or unsubstituted 5-(aryl-sulfonyl)-4,5-dihydro-1H-pyrazolo[4,3-c]quinolines or 5-(heteroaryl-sulfonyl)-4,5-dihydro-1H-pyrazolo[4,3-c]quinolines. Compared to known methods, the current processes are scalable, cost-efficient (e.g., starting materials are more readily available and the number of isolation procedures are minimized, e.g., chromatography steps are omitted), generally more reliable and safer to perform (e.g., diazotization steps are omitted) and are characterized by significantly reduced environmental impact (e.g., less solvents, reduced amount of metal catalysts). Overall, the current processes are higher yielding and result in a final product, which is of greater chemical and chiral purity.

For example, known methods involve the use of a copper-mediated cyclization reaction, which requires a large amount of copper reagent. Copper catalyzed carbon-nitrogen bond-formation reactions that can be performed with a reduced amount of copper, e.g., by employing organic copper ligands such as cyclohexyldiamines (see, e.g., Buchwald et al., U.S. Pat. No. 6,759,554 and Buchwald et al., U.S. Pat. No. 7,115,784, both disclosures of which are incorporated herein by reference in their entirety) and N-alkylglycines (see, e.g., Deng et al., Tetrahedron Letters 2005, 46: 7295-7298, incorporated herein by reference) have been described. However, such methods have not been applied to affect intra-molecular cyclizations involving an amide or a sulfonamide group.

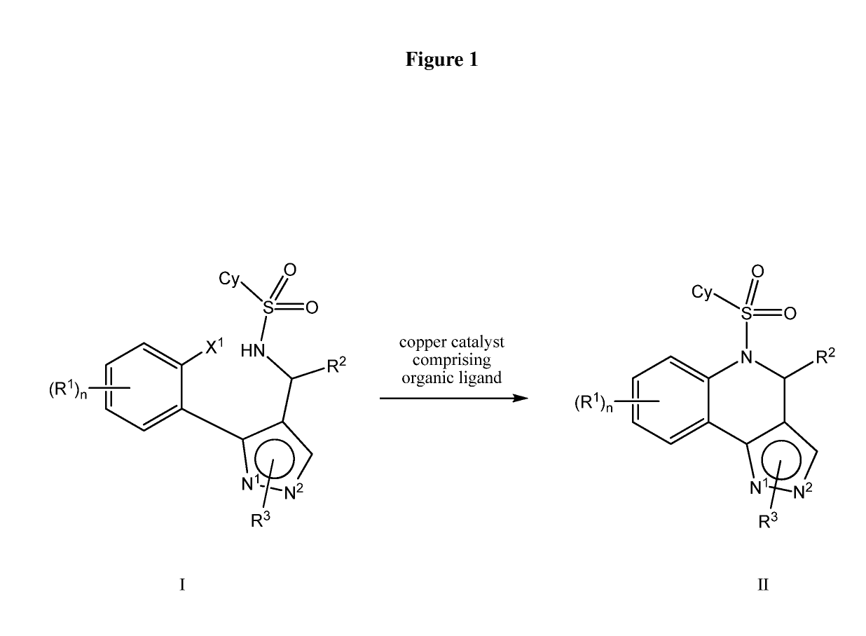

In one example, the disclosure provides a method of affecting an intra-molecular cyclization reaction, wherein a bond is formed between a nitrogen atom of a sulfonamide group and a carbon atom, which is part of an aromatic or a hetero-aromatic ring, thereby displacing a leaving group, such as a halogen atom. An exemplary method leads to a tricyclic core structure comprising a tertiary sulfonamide moiety. Exemplary core structures, which can be prepared using a method of the invention include 5-(sulfonyl)-4,5-dihydro-2H-pyrazolo[4,3-c]quinoline and 5-(sulfonyl)-4,5-dihydro-1H-pyrazolo[4,3-c]quinoline. An exemplary intra-molecular cyclization reaction according to a method of this disclosure is illustrated in FIG. 1 and Scheme 1, below.

### Method 1

An exemplary method includes (i) contacting a first molecule having a structure according to Formula (I), or a salt, solvate, tautomer, mixture of tautomers, stereoisomer or mixture of stereoisomers thereof, with a catalyst that includes copper (e.g., Cu(0), Cu(I) or Cu(II)) and at least one organic ligand (e.g., a 1,2-diamine). The reactants are contacted under conditions sufficient to form a second molecule having a structure according to Formula (II), or a salt, solvate, tautomer, mixture of tautomers, stereoisomer or mixture of stereoisomers thereof. The catalyst can be formed by contacting a copper ion (i.e., copper salt, such as CuI) or a copper complex with an organic ligand. Exemplary organic ligands, which are useful in the methods of the invention, are described herein below. In one example, the ligand is capable of forming a complex with the copper. In one example, the above method is a large-scale method.

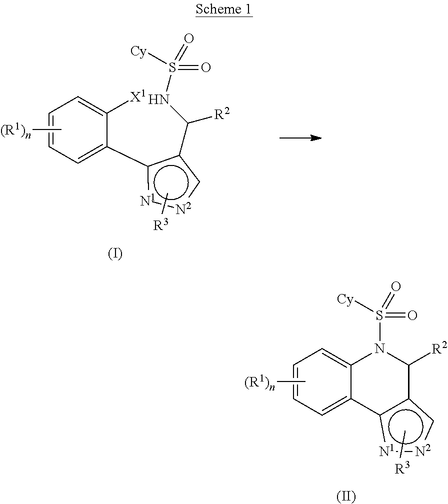

In Formula (I), X¹ represents a leaving group (e.g., a halogen). In one example, X¹ is a member selected from I, Br, Cl, F, tosylate (4-CH₃—C₆H₄—S(O)₂—O—) and mesylate (CH₃—S(O)₂—O—). In another example, X¹ is I, Br or F. In yet another example, X¹ is Br. In a further example, X¹ is F. In Formula (I) and Formula (II), N¹ and N² are nitrogen atoms of a pyrazole ring, and n is an integer selected from 0 to 4.

In Formula (I) and Formula (II), each R¹ is independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶, and S(O)₂R⁶, wherein the alkyl, alkenyl, alkynyl, heteroalkyl, cycloalkyl, heterocycloalkyl, aryl and heteroaryl is optionally substituted, e.g., with from 1 to 5 (e.g., from 1 to 3) substituents independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In one embodiment, R¹ is selected from halogen, CN, C₁-C₄ alkyl, C₁-C₄ haloalkyl, or C₁-C₄ haloalkoxy.

In Formula (I) and Formula (II), R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring. R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.

In one example, in Formula (I) and (II), each R¹ is a member independently selected from substituted or unsubstituted C₁-C₃ alkyl (e.g., methyl, ethyl or propyl), halogen (e.g., F, Cl or Br) and CN. In another example, n is 1 or 2 and each R¹ is halogen. In yet another example, n is 1 or 2 and each R¹ is F. In a further example, n is 1 and R¹ is F. In another example, n is 2 and each R¹ is F.

In Formula (I) and Formula (II), R² is selected from H, alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), wherein the alkyl, alkenyl, alkynyl, haloalkyl, cycloalkyl, heterocycloalkyl, aryl, and heteroaryl is optionally substituted with from 1 to 5 (e.g., from 1 to 3) substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In one example, R² is selected from C₁-C₄ alkyl, C₃-C₆ cycloalkyl, and aryl, which are all optionally substituted. In one example, R² is optionally substituted C₃-C₆-cycloalkyl. In another example, R² is optionally substituted cyclopropyl. In yet another example, R² is cyclopropyl.

In Formula (I) and Formula (II), R³ is an amino protecting group covalently bonded to either N¹ or N² of the pyrazole ring. Amino protecting groups are known to those of skill in the art and exemplary amino protecting groups are described herein. In one example, R³ is selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), cycloalkyl (e.g., C₃-C₆-cycloalkyl), heterocycloalkyl (e.g., 3- to 8-membered heterocycloalkyl), aryl (e.g., phenyl), and heteroaryl (e.g., 5- or 6-membered heteroaryl), wherein the alkyl, alkenyl, alkynyl, haloalkyl, cycloalkyl, heterocycloalkyl, aryl, and heteroaryl is optionally substituted with from 1 to 5 (e.g., from 1 to 3) substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In another example, R³ is selected from optionally substituted C₁-C₆ alkyl, optionally substituted C₁-C₆ alkenyl, and optionally substituted C₁-C₆ alkynyl. In yet another example, R³ is tert-butyl or benzyl. In another example, R³ is tert-butyl. In yet another example, R³ is a silyl ether, such as 2-(trimethylsilyl)ethoxymethyl (SEM) ether; or an alkoxymethyl ether, such as methoxymethyl (MOM) ether, tert-butoxymethyl (BUM) ether, benzyloxymethyl (BOM) ether, or methoxyethoxymethyl (MEM) ether. In yet another example, R³ is a s2-(trimethylsilyl)ethoxymethyl (SEM ether) or methoxymethyl (MOM ether). In one example, R³ in Formula (I) or (II) is covalently bonded to N¹ of the pyrazole ring. In another example, R³ is covalently bonded to N² of the pyrazole ring.

In Formula (I) and Formula (II), Cy is a member selected from cycloalkyl (e.g., C₃-C₆-cycloalkyl), heterocycloalkyl (e.g., 3- to 8-membered heterocycloalkyl), aryl (e.g., phenyl) and heteroaryl (e.g., 5- or 6-membered heteroaryl), wherein the cycloalkyl, heterocycloalkyl, aryl or heteroaryl is optionally substituted with from 1 to 5 substituents, wherein each substituent is independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, N¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In one embodiment, Cy is aryl or heteroaryl, each of which is optionally substituted with halogen, C₁-C₄ haloalkyl, or C₁-C₄ haloalkoxy. In one example, Cy is optionally substituted phenyl. In another example, Cy is optionally substituted pyridyl (e.g., pyridin-3-yl). In yet another example, Cy is haloalkyl-substituted phenyl. In a further example, Cy is haloalkyl-substituted pyridyl. In yet another example, Cy is CF₃-substituted phenyl or CF₃-substituted pyridyl. In another example Cy is phenyl or pyridyl, wherein the phenyl or pyridyl is optionally substituted with 1 to 4 substituents selected from halogen, C₁-C₄ haloalkyl (e.g., —CF₃), and C₁-C₄ haloalkoxy (e.g., —OCF₃).

In Formula (I) and Formula (II), each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring. R¹⁶ is selected from acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl, and 3- to 8-membered heterocycloalkyl.

It is also contemplated that in Formula (I) and Formula (II), the phenyl ring that carries X¹ and R¹ can be replaced with a 6-membered heteroaromatic ring comprising from 1 to 3 nitrogen atoms. Exemplary heteroaromatic rings include pyridine and pyrimidine.

In one example, X¹ in Formula (I) is Br. In another example, the molecule of Formula (I) has a structure according to Formula (Ia):

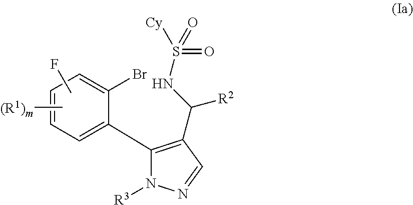

or a salt or solvate thereof, wherein Cy, R¹, R² and R³ are defined as for Formula (I), above (or any embodiment thereof), and m is an integer selected from 0 to 3. In one example m is 0 or 1. In another example m is 0 or 1 and each R¹ is halogen.

In another example, the molecule of Formula (I) has a structure according to Formula (Ib):

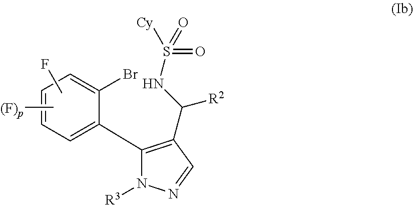

or a salt or solvate thereof, wherein Cy, R² and R³ are defined as for Formula (I) and p is an integer selected from 0 to 3. In one example p is 0 or 1.

In another example, the molecule of Formula (I) has the structure according to Formula (Ic):

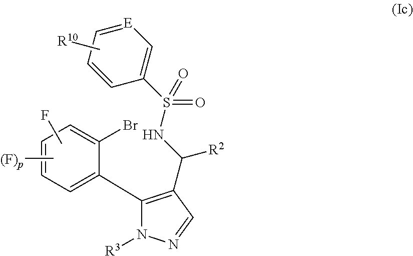

or a salt or solvate thereof, wherein R² and R³ are defined as for Formula (I), p is an integer selected from 0 to 3, and E is CH or N.

In Formula (Ic), R¹⁰ is a member selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR²⁴, SR²⁴, NR²⁴R²⁵, C(O)R²⁶, C(O)NR²⁴R²⁵, OC(O)NR²⁴R²⁵, C(O)OR²⁴, NR²⁷C(O)R²⁶, NR²⁷C(O)OR²⁴, NR²⁷C(O)NR²⁴R²⁵, NR²⁷C(S)NR²⁴R²⁵, NR²⁷S(O)₂R²⁶, S(O)₂NR²⁴R²⁵, S(O)R²⁶ and S(O)₂R²⁶, wherein R²⁴, R²⁵ and R²⁷ are independently selected from H, acyl, C₁-C₆-alkyl, C₁-C₆ haloalkyl, 2- to 6-membered heteroalkyl, aryl, 5- or 6-membered heteroaryl, C₃-C₈ cycloalkyl and 3- to 8-membered heterocycloalkyl, wherein R²⁴ and R²⁵, together with the nitrogen atom to which they are bound are optionally joined to form a 5- to 7-membered heterocyclic ring. R²⁶ is independently selected from acyl, C₁-C₆-alkyl, C₁-C₆ haloalkyl, 2- to 6-membered heteroalkyl, aryl, 5- or 6-membered heteroaryl, C₃-C₈ cycloalkyl and 3- to 8-membered heterocycloalkyl. In one example, R¹⁰ is selected from C₁-C₄ alkyl, C₁-C₄ haloalkoxy, and C₁-C₄ haloalkyl. In another example, in Formula (Ic), R² is cyclopropyl.

In another example, the molecule of Formula (I) has the structure according to Formula (Id):

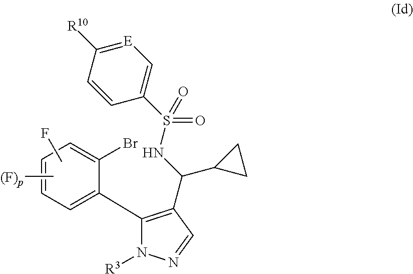

or a salt or solvate thereof, wherein R³, p, E, and R¹⁰ are defined as above. In one example, in Formula (Ic) or Formula (Id), R¹⁰ is C₁-C₄ haloalkyl or C₁-C₄ haloalkoxy. In yet another example, R¹⁰ is CF₃.

In another example, the molecule of Formula (I) has the structure according to Formula (Ie):

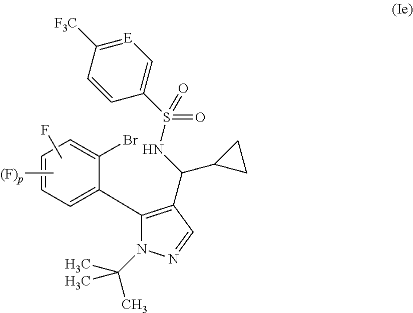

or a salt or solvate thereof, wherein p and E are defined as above. In one example, in Formula (Ic), (Id) or (Ie), E is CH. In another example, in Formula (Ic), (Id) or (Ie), E is N. In another example, in Formula (Ib), (Ic), (Id) or (Ie), the integer p is 0 or 1.

In another example, the molecule of Formula (I) has a structure selected from:

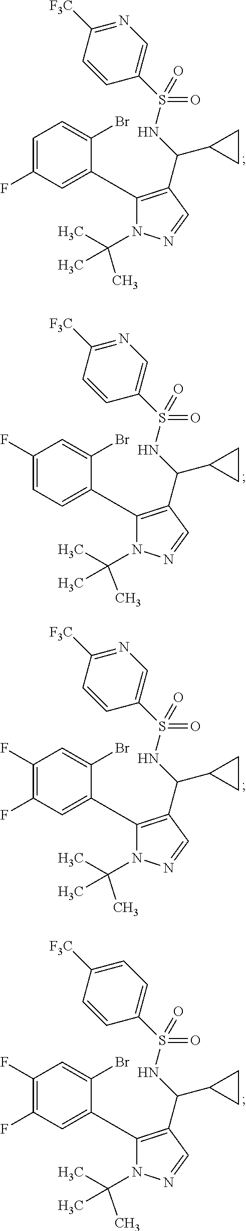

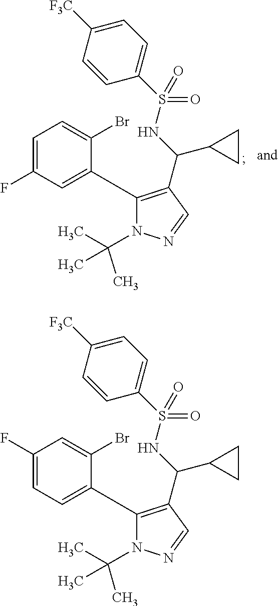

or a salt or solvate thereof.

### Copper Catalyst

### Formation of the Catalyst

In one example, the catalyst of Scheme 1 is formed in situ. For example, the molecule of Formula (I) is contacted with a copper source (described below), such as a copper salt (e.g., CuI or CuCl) and the resulting mixture is then contacted with one or more organic ligand, described herein below. In another example, the molecule of Formula (I) is first contacted with the ligand and the resulting mixture is then contacted with a copper source. In a further example, the catalyst is formed prior to contacting with the molecule of Formula (I). For example, the copper source can be contacted with at least one ligand under conditions sufficient to form a “pre-formed” catalyst. The pre-formed catalyst is then contacted with the molecule of Formula (I).

### Amount of Copper

The amount of copper used in the methods of the invention is typically less than 2 equivalents (less than 200 mol % (mole percent)) relative to the non-cyclized starting material. In one example, the copper, which is used for the conversion as shown in Scheme 1, is present in the reaction mixture in an amount equivalent to between about 0.01 mol % and about 100 mol % relative to the amount of the first molecule of Formula (I). In another example, the copper is present in the reaction mixture in an amount equivalent to between about 0.01 mol % and about 30 mol % relative to the amount of the first molecule of Formula (I). In another example, the copper is present in an amount equivalent to between about 0.1 mol % and about 50 mol % relative to the amount of the first molecule. In yet another example, the copper is present in an amount equivalent to between about 0.1 mol % and about 30 mol %, between about 0.1 mol % and about 25 mol %, between about 0.1 mol % and about 20 mol %, between about 0.1 mol % and about 15 mol % or between about 0.1 mol % and about 10 mol % relative to the amount of the first molecule. In a further example, the copper is present in an amount equivalent to between about 0.5 mol % and about 20 mol %, between about 0.5 mol % and about 15 mol % or between about 0.5 mol % and about 10 mol % relative to the first molecule. In yet another example, the copper is present in an amount equivalent to between about 1 mol % and about 20 mol %, between about 1 mol % and about 15 mol %, between about 1 mol % and about 10 mol %, between about 1 mol % and about 8 mol %, between about 1 mol % and about 6 mol % or between about 1 mol % and about 4 mol % relative to the first molecule. In a further example, the copper is present in an amount equivalent to between about 1 mol % and about 3 mol % or between about 1 mol % and about 2 mol % relative to the first molecule. In a particular example, the copper is present in an amount equivalent to about 2 mol % relative to the first molecule.

### Copper Source

The copper source can be any copper reagent or mixture of copper reagents. The copper in each reagent can have any oxidative state. The oxidative state of the copper can change upon forming a complex with the one or more ligand. In one embodiment the copper source is a copper salt or a mixture of copper salts. In one example, the copper in the copper salt is Cu(I). In another example, the copper in the copper salt is Cu(II). Exemplary copper salts useful in the methods of the invention include copper halides, such as CuI, CuCl and CuBr. Other suitable copper sources include copper oxides. In a particular embodiment, the copper source comprises CuI. In another particular embodiment, the copper source consists essentially of CuI.

### Organic Ligand

The copper catalyst used in the methods of the invention includes at least one organic ligand. The ligand of the copper catalyst can be any organic ligand. Exemplary organic ligands are capable of forming a complex with a copper ion. Copper-complexing ligands are known in the art. See, e.g., Buchwald et al., U.S. Pat. No. 6,759,554 and Buchwald et al., U.S. Pat. No. 7,115,784, the disclosures of which are incorporated herein in their entirety for all purposes. Exemplary ligands include 1,2-diamines and N,N-dialkylsalicylamides. In one example, the ligand is a member selected from N¹,N²-dialkylcyclohexane-1,2-diamine (e.g., N¹,N²-dimethylcyclohexane-1,2-diamine), N¹,N²-dialkylethane-1,2-diamine (e.g., N¹,N²-dimethylethane-1,2-diamine), N¹, N²-tetraalkylethane-1,2-diamine (e.g., N¹,N¹, N²,N²-tetramethylethane-1,2-diamine) and N,N-dialkylsalicylamides (e.g., N,N-diethylsalicylamide). In one example, the ligand is preferably not acetate (CH₃COO⁻), e.g., is not derived from CsOAc. In another example, the ligand is preferably not an amino acid. For example, in a preferred embodiment, the ligand is not N-alkylglycine (e.g., N-methylglycine) or N,N-dialkylglycine (e.g., N,N-dimethylglycine).

The ligand, which is used in the methods of the invention can be present in any amount. The amount of organic ligand present in the reaction mixture will typically be determined by the amount of copper and the amount of starting material used in the reaction. In one example, the ligand is present in an amount equivalent to between about 0.1 mol % and about 150 mol % relative to the first molecule of Formula (I). In another example, the ligand is present in an amount equivalent to between about 1 mol % and about 100 mol % relative to the first molecule. In yet another example, the ligand is present in an amount equivalent to between about 1 mol % and about 90 mol %, between about 1 mol % and about 80 mol %, between about 1 mol % and about 75 mol %, between about 1 mol % and about 70 mol %, between about 1 mol % and 65 mol %, between about 1 mol % and about 60 mol %, between about 1 mol % and about 55 mol % or between about 1 mol % and about 50 mol % relative to the first molecule. In a further example, the organic ligand is present in an amount equivalent to between about 1 mol % and about 45 mol %, between about 1 mol % and about 40 mol %, between about 1 mol % and about 35 mol %, between about 1 mol % and about 30 mol %, between about 1 mol % and about 25 mol % or between about 1 mol % and about 20 mol % relative to the first molecule. In yet another example, the ligand is present in an amount equivalent to between about 2 mol % and about 20 mol %, between about 2 mol % and about 18 mol %, between about 2 mol % and about 16 mol %, between about 2 mol % and about 14 mol %, between about 2 mol % and about 12 mol % or between about 2 mol % and about 10 mol % relative to the first molecule. In another example, the ligand is present in an amount equivalent to between about 5 mol % and about 15 mol % relative to the amount of the first molecule. In a particular example, the ligand is present in an amount equivalent to about 10 mol % relative to the amount of the first molecule of Formula (I). In yet another example, the ligand is present in an amount between about 1 equivalent and about 10 equivalents relative to the copper source. In a further example, the ligand is present in an amount equivalent to between about 2 equivalents and about 6 equivalents relative to the copper source. In another particular example, the ligand is present in an amount equivalent to about 5 equivalents relative to the copper source.

### Base

In one example, the reactants in Scheme 1, above are contacted in the presence of a base. The base can be any base and is preferably a Bronsted base, such as those known to be useful in metal-catalyzed cross-coupling reactions. Exemplary bases include salts of organic and inorganic anions, such as carbonates, phosphates, acetates and the like. In a particular example, the base is potassium carbonate (K₂CO₃), sodium carbonate (Na₂CO₃), cesium carbonate (Cs₂CO₃), potassium phosphate (K₂PO₄), sodium phosphate (Na₂PO₄) and the like. In one example, the base is preferably not cesium acetate (CsOAc). The base can be present in the reaction mixture in any amount. In one example, the base is used in an amount equivalent to between about 1 equivalent (100 mol %) and about 5 equivalents relative to the molecule of Formula (I). In another example, the base is used in an amount equivalent to between about 1.5 equivalents (150 mol %) and about 3.0 equivalents relative to the first molecule of Formula (I). In yet another example, the base is used in an amount equivalent to between about 1.5 equivalents and about 2.0 equivalents relative to the first molecule of Formula (I). In a particular example, the base is used in an amount equivalent to about 1.7 equivalents (170 mol %) relative to the first molecule of Formula (I).

### Solvent, Reaction Temperature and Reaction Time

In one example, the reactants in Scheme 1 are contacted in the presence of a solvent. The term “solvent” is intended to refer to a liquid that dissolves a solid, liquid, or gaseous solute to form a solution. Common solvents are well known in the art and include but are not limited to, water; saturated aliphatic hydrocarbons, such as pentane, hexane, heptanes, and other light petroleum; aromatic hydrocarbons, such as benzene, toluene, xylene (i.e., ortho-, meta- and para-xylene), etc.; halogenated hydrocarbons, such as dichloromethane, chloroform, carbon tetrachloride, etc.; aliphatic alcohols, such as methanol, ethanol, propanol, etc., ethers, such as diethyl ether, dipropyl ether, dibutyl ether, tetrahydrofuran, dioxane, etc.; ketones, such as acetone, ethyl methyl ketone, etc.; esters, such as methyl acetate, ethyl acetate, etc.; nitrogen-containing solvents, such as formamide, N,N-dimethylformamide, acetonitrile, pyridine, N-methylpyrrolidone, quinoline, nitrobenzene, etc.; sulfur-containing solvents, such as carbon disulfide, dimethyl sulfoxide, sulfolane, etc.; phosphorus-containing solvents, such as hexamethylphosphoric triamide, etc. The term solvent includes a combination of two or more solvents unless clearly indicated otherwise. A particular choice of a suitable solvent will depend on many factors, including the nature of the solvent and the solute to be dissolved and the intended purpose, for example, what chemical reactions will occur in the solution, and is generally known in the art.

The solvent used herein can be any solvent. The term “solvent” includes mixtures of at least two different solvents. Exemplary solvents, which are useful in the methods of the invention, include aromatic solvents, such as xylene (i.e., ortho-, meta- and para-xylene), toluene and mixtures thereof. In one example, the solvent has a boiling point of at least about 100° C., at least about 120° C. or at least about 130° C. The reaction mixture can optionally be pressurized enabling the use of a greater variety of solvents with lower boiling points. The reaction mixture is typically heated to a temperature between about 100° C. and about 160° C. In one example, the reaction mixture is heated to between about 110° C. and 140° C. In another example, the reaction mixture is heated to about 135° C. In a particular example, the solvent is toluene and the reaction mixture is heated to about 135° C. while the reaction mixture is pressurized to between about 1.5 and about 2.5 bar.

In one example, the reaction mixture is heated for a period between about 1 hour (h) and about 100 h. In another example, the reaction mixture is heated for a period between about 2 h and about 72 h. In yet another example, the reaction mixture is heated for a period between about 2 h and about 36 h, between about 2 h and about 24 h or between about 2 h and about 12 h. In a further example, the reaction mixture is heated for a period between about 2 h and about 10 h, between about 2 h and about 8 h or between 2 h and about 6 h. In a particular example, the reaction mixture is heated to between about 100° C. and about 150° C. for a period between about 2 h and about 12 h.

### Reaction Yield

In one example, the second compound of Formula (II) in Scheme 1 is formed from the first molecule of Formula (I) with a reaction yield (e.g., isolated yield, mol/mol) between about 50% and about 100%, between about 60% and about 100%, between about 70% and about 100%, between about 80% and about 100% or between about 90% and about 100% (mol/mol) relative to the amount of starting material (first molecule of Formula (I)) used in the reaction. In another example, the second molecule of Formula (II) in Scheme 1 is formed with a reaction yield of at least about 80%, at least about 90%, at least about 95%, at least about 96%, at least about 97%, at least about 98% or at least about 99% (mol/mol) relative to the first molecule. Reaction yields can alternatively be determined using various chromatography methods (e.g., LC/MS). The ratio between the amount of starting material (first molecule) and the amount of product (second molecule) in the final reaction mixture can be determined, for example, using “area under the curve” (AUC) values.

Method 1 provide a series of advantages over known methods. For example, as a result of using a copper catalyst, which includes at least one organic ligand, such as DMEDA, the amount of copper needed for the intra-molecular cyclization reaction, is significantly reduced, making the current process more cost effective and environmentally friendly. While known methods employ between about 2.5 and 10 equivalents (e.g., 5 equivalents) of catalytic copper, the current process requires less than 2 equivalents, and preferably less than 1 equivalent of catalytic copper.

### Side-Product Formation

In one example, the above described method of cyclization is associated with reaction yields that are significantly higher than those found for known methods. One reason for the improved yield is the significantly reduced amount of de-brominated side-product formed during the cross-coupling reaction. Metal-catalyzed cross-coupling reactions are commonly associated with a side-reaction, in which the leaving group (e.g., a halogen atom) is removed (e.g., de-halogenation) while the intended bond-formation does not take place. For example, a de-halogenation reaction competes with the cyclization (bond-formation) reaction. An exemplary de-bromination reaction, which can occur when treating compounds of Formula (I) with a metal catalyst, is shown in Scheme 2, below:

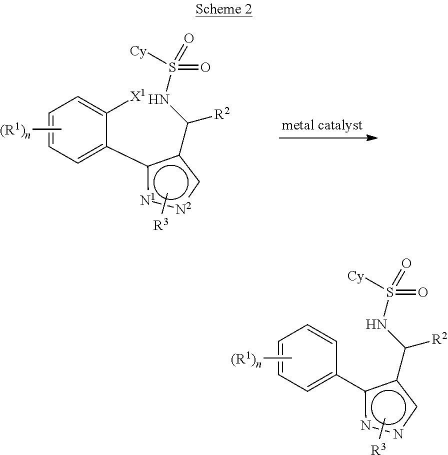

In Scheme 2, X¹, Cy, n, R¹, R², and R³ are defined as for Formula (I). In one example in Scheme 2, X¹ is Br.

The cyclization method of the invention, in which a copper catalyst is used that incorporates at least one organic ligand (e.g., at least one diamine-ligand), produces a final reaction mixture, in which the concentration of de-halogenated (e.g., de-brominated) impurities in the crude product is unexpectedly low (e.g., less than 1% AUC). Example 14 describes a known cyclization procedure employing CuI (2 eq) and CsOAc (5 eq) without an organic ligand. The crude reaction mixture thus produced includes about 13% (AUC) of a de-brominated side-product. Contrarily, when using a cyclization procedure of the invention (see, e.g., Example 4.4.), the concentration of de-brominated side-product in the crude reaction mixture is below the level of detection (e.g., less than 1% AUC).

When using the above described method, the second molecule of Formula (II) is formed with an improved reaction yield, while the amount of a de-halogenated (e.g., de-brominated) impurity formed during the reaction is reduced. In one example, the de-halogenated (e.g., de-brominated) impurity is formed in an amount equivalent to not more than about 10% (mol/mol) relative to the first molecule of Formula (I) (starting material) or not more than about 10% AUC in the crude product mixture. In another example, the de-halogenated impurity is formed in an amount equivalent to not more than about 8%, not more than about 6% or not more than about 4% (mol/mol) relative to the first molecule of Formula (I) or not more than about 8, 6 or 4% AUC in the crude product mixture. In yet another example, the de-halogenated impurity is formed in an amount equivalent to not more than about 3.8%, not more than about 3.6%, not more than about 3.4%, not more than about 3.2% or not more than about 3.0% (mol/mol) relative to the first molecule of Formula (I) or not more than about 3.8, 3.6., 3.4, 3.2 or about 3% AUC in the crude product mixture. In a further example, the de-halogenated impurity is formed in an amount equivalent to not more than about 2.8%, not more than about 2.6%, not more than about 2.4%, not more than about 2.2% or not more than about 2.0% (mol/mol) relative to the first molecule of Formula (I) or not more than about 2.8, 2.6, 2.4, 2.2 or 2% AUC in the crude product mixture. In another example, the de-halogenated impurity is formed in an amount equivalent to not more than about 1.8%, not more than about 1.6%, not more than about 1.4%, not more than about 1.2% or not more than about 1.0% (mol/mol) relative to the first molecule of Formula (I) or not more than about 1.8, 1.6, 1.4, 1.2 or 1% AUC in the crude product mixture. In yet another example, the de-halogenated impurity is formed in an amount equivalent to not more than about 0.8%, not more than about 0.6%, not more than about 0.4%, not more than about 0.2% or not more than about 0.1% (mol/mol) relative to the first molecule of Formula (I) or not more than about 0.8, 0.6, 0.4, 0.2 or about 0.1% AUC in the crude product mixture.

In one example, the above described method of cyclization reduces the formation of an aromatized side product having the formula:

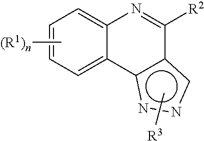

wherein n, R¹, R² and R³ are defined as for Formula (I), above. In one example, the aromatized side product is formed in an amount equivalent to not more than about 5% (mol/mol) relative to the first molecule of Formula (I) (starting material) or not more than about 5% AUC in the crude product mixture. In another example, the aromatized side product is formed in an amount equivalent to not more than about 4%, not more than about 3% or not more than about 2% (mol/mol) relative to the first molecule of Formula (I) or not more than about 4, 3 or 2% AUC in the crude product mixture. In yet another example, the aromatized side product is formed in an amount equivalent to not more than about 1%, not more than about 0.8%, not more than about 0.6%, not more than about 0.4%, not more than about 0.2% or not more than about 0.1% (mol/mol) relative to the first molecule of Formula (I) or not more than about 1, 0.8, 0.6, 0.4, 0.2 or 0.1% AUC in the crude product mixture.

In a particular example, the second molecule of Formula (II) is formed with a reaction yield of between about 80% and about 100% (mol/mol) relative to the first molecule of Formula (I), while a de-halogenated (e.g., de-brominated) impurity is formed in an amount equivalent to not more than about 10%, about 8%, about 6%, about 4%, about 2% or about 1% (mol/mol) relative to the first molecule of Formula (I). In another particular example, the second molecule of Formula (II) is formed with a reaction yield of between about 80% and about 100% (mol/mol) relative to the first molecule of Formula (I), while the de-brominated impurity is formed in an amount equivalent to not more than about 2% (mol/mol) relative to the first molecule of Formula (I) (or not more than about 2% AUC in the crude product mixture); and the aromatized side product is formed in an amount equivalent to not more than about 1% (mol/mol) relative to the first molecule of Formula (I) (or not more than about 1% AUC in the crude product mixture).

### Other Process Steps

The above described method of cyclizing molecules of Formula (I) can further include one or more of the following process steps. In one example, the method further includes: purifying the second molecule of Formula (II). The second molecule can be purified, e.g., by crystallization or precipitation. An exemplary method of purification includes: (a) heating the second molecule in a mixture containing alcohol (e.g., methanol) and water, thereby forming a solution; (b) cooling the solution of step (a), thereby forming a precipitate (e.g., crystals) of the second molecule; and, optionally, (c) isolating the precipitate of step (b). In one example, the mixture of step (a) includes water in an amount equivalent to between about 1% (v/v) and about 50% (v/v). In another example, the mixture of step (a) includes water in an amount equivalent to between about 5% (v/v) and about 20% (v/v). In a further example, the mixture of step (a) includes water in an amount equivalent to between about 8% (v/v) and about 12% (v/v). In another example, the mixture of step (a) includes about 10% water (v/v). In another example, the mixture of step (a) is methanol/water of about 10:1 (v/v).

In one example, the above described method results in a compound of Formula (II) with improved chiral purity compared to the chiral purity before crystallization or precipitation. In one example the above procedure involving methanol and water results in chiral purity after crystallization/precipitation between about 90% and about 100% (AUC on a chiral column). In another example, chiral purity after crystallization/precipitation is between about 95% and about 100%. In yet another example, chiral purity after crystallization/precipitation is greater than 95% AUC (or 90% ee), greater than 96%, greater than 97%, greater than 98% or greater than 99%.

In another example, the method further includes: removing the amino protecting group from the second molecule, thereby forming a third molecule having a structure according to Formula (A):

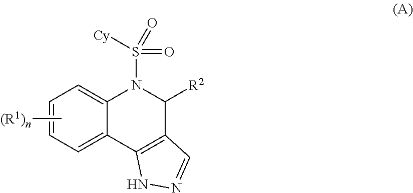

or a salt, solvate, tautomer, mixture of tautomers, stereoisomer or mixture of stereoisomers thereof, wherein Cy, n, R¹ and R² are defined as for Formula (I), above.

In one example, removing the amino protecting group is accomplished using acid. In another example, the amino protecting group is removed using aqueous formic acid and (optionally) heat, thereby forming an acidic reaction mixture. The method can further include (e.g., after the deprotection is complete): contacting (i.e., mixing) the acidic reaction mixture with a sufficient amount of water, thereby forming a precipitate; and isolating the precipitated or crystallized product, e.g., by filtration. Precipitation of the reaction product using water significantly simplifies the overall process and lowers associated costs, when compared with a conventional work-up procedure, i.e., stripping the formic acid and performing a customary extraction procedure using organic solvents. Simple precipitation using water can also reduce the presence of certain by-products, or reduce the formation of certain by-products, such as N²-substituted pyrazoles.

The above method (method 1) can further include: purifying the third molecule of Formula (A) after deprotection. An exemplary purification method for the third molecule includes: (a) forming a solution of the third molecule in a suitable solvent (e.g., ethanol); (b) contacting (i.e., mixing) the solution of step (a) with a sufficient amount of water, thereby forming a precipitate of the third molecule; and optionally (c) isolating the precipitate (e.g., using filtration). In one example, the water of step (b) is cooled to a temperature of about 10° C. or less. In another example, the water of step (b) is cooled to a temperature of 5° C. or less. In yet another example, the water of step (b) is cooled to a temperature between about 1° C. and about 5° C. In one example, the above described method results in a compound of Formula (A) with improved chemical purity compared to the chemical purity before crystallization or precipitation. In one example the above precipitation/crystallization procedure results in chemical purity after crystallization/precipitation between about 90% and about 100%. In another example the above precipitation or crystallization procedure results in chemical purity between about 98% and about 100%.

The above method of cyclizing compounds of Formula (I) can further include processing steps relating to the making of compounds of Formula (I) as outlined hereinbelow (e.g., methods 3-5).

### Method 2

The current disclosure further provides an intramolecular cyclization method that does not require a metal catalyst. As such, this method occurs in the absence of a metal catalyst. What is meant by “in the absence of” is that there may be an amount of metal or metal catalyst is only present in trace amounts in the reaction vessel. This method is particularly useful when the aromatic ring involved in the cyclization is substituted with at least two electron withdrawing groups, e.g., halogen atoms (e.g., at least 3 halogen atoms including the leaving group X¹).

An exemplary method according to this embodiment includes: (i) contacting a first molecule having a structure according to Formula (III):

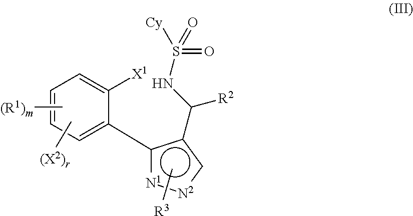

or a salt or solvate thereof, wherein N¹, N², Cy, R¹, R², R³ and X¹ are defined as for Formula (I) (or any of its embodiments), m is an integer selected from 0 to 3, each X² is independently selected from halogen (e.g., F, Cl, Br) and another electron withdrawing group known to those of skill in the art, and r is an integer selected from 1 to 4 (e.g., r is selected from 2 to 4), provided that the sum of m and r is not greater than 4, with a base under conditions (e.g., heat) sufficient to form a second molecule having a structure according to Formula (IV):

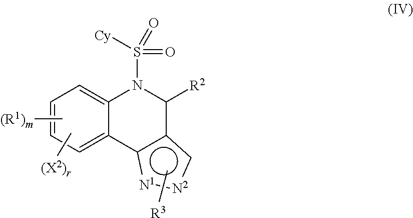

or a salt or solvate thereof, wherein N¹, N², Cy, R¹, R², R³, m, r and X² are defined as above. In one example, in Formula (III) and Formula (IV), R³ is covalently bonded to N¹ of the pyrazole ring. In another example, in Formula (III) and Formula (IV), R³ is covalently bonded to N² of the pyrazole ring. Suitable bases that are useful in the above method are described hereinbelow.

In one example in Formula (III) and Formula (IV), the integer r is 1. In another example, r is 2. In yet another example, r is 2 and both X² are independently selected from halogen. In a further example in Formula (III) and Formula (IV), r is 2 and both X² are F. In another example r is 1 and X² is F.

In one example in Formula (III), X¹ is a member selected from I, Br, Cl, F, tosylate and mesylate. In another example in Formula (III), X¹ is F. In another example, X¹ is F, r is 2 and both X² are independently selected from halogen (e.g., F, Cl or Br). In a further example in Formula (III), r is 1, and X¹ and X² are both F. In another example in Formula (III), r is 2, X¹ is F, and each X² is F.

It is also contemplated that in Formula (III) and Formula (VI), the phenyl ring that carries X¹, R¹ and X² can be replaced with a 6-membered heteroaromatic ring comprising from 1 to 3 nitrogen atoms. Exemplary heteroaromatic rings include pyridine and pyrimidine.

In one example, the molecule of Formula (III) has a structure according to Formula (IIIa):

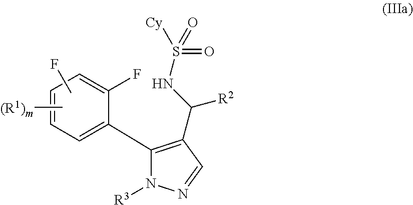

or a salt or solvate thereof, wherein Cy, R¹, R² and R³ are defined as for Formula (I), above, and m is an integer selected from 0 to 3. In one example m is 0 or 1. In another example m is 1 and R¹ is halogen.

In another example, the molecule of Formula (III) has a structure according to Formula (IIIb):

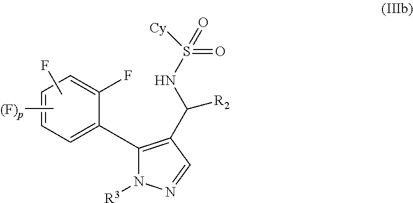

or a salt or solvate thereof, wherein Cy, R² and R³ are defined as for Formula (I) and p is an integer selected from 0 to 3. In one example p is 0 or 1.

In another example, the molecule of Formula (III) has the structure according to Formula (IIIc):

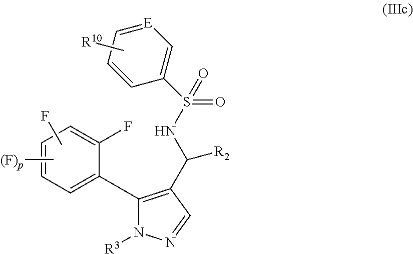

or a salt or solvate thereof, wherein R² and R³ are defined as for Formula (I), p is an integer selected from 0 to 3, and E is CH or N. In Formula (IIIc), R¹⁰ is defined as for Formula (Ic), above. In one example, R¹⁰ in Formula (IIIc) is selected from halogen (e.g., F or Cl), CN, C₁-C₄ alkyl (e.g., methyl), C₁-C₄ haloalkoxy, and C₁-C₄ haloalkyl (e.g., CHF₂ or CF₃).

In another example, the molecule of Formula (III) has the structure according to Formula (IIId):

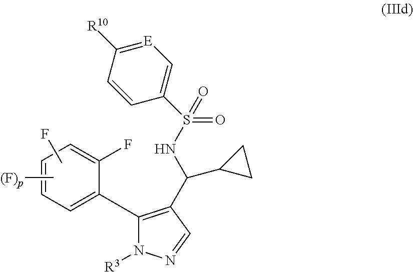

or a salt or solvate thereof, wherein R³, p, E, and R¹⁰ are defined as above. In one example, in Formula (IIIc) or Formula (IIId), R¹⁰ is C₁-C₄ haloalkyl. In yet another example, R¹⁰ is CF₃.

In another example, the molecule of Formula (III) has the structure according to Formula (IIIe):

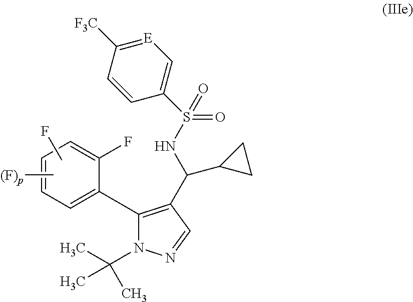

or a salt or solvate thereof, wherein p and E are defined as above. In one example, in Formula (IIIc), (IIId) or (IIIe), E is CH. In another example, in Formula (IIIc), (IIId) or (IIIe), E is N. In another example, in Formula (IIIb), (IIIc), (IIId) or (IIIe), the integer p is 0 or 1. In another example, in Formula (IIIb), (IIIc), (IIId) or (IIIe), the integer p is 1.

In another example, the molecule of Formula (III) has a structure selected from:

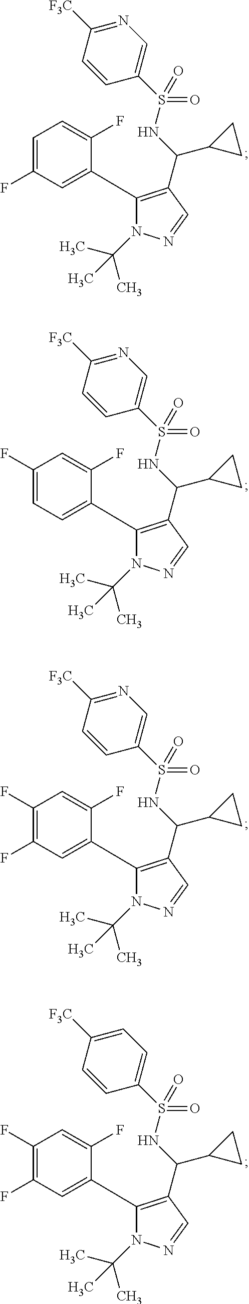

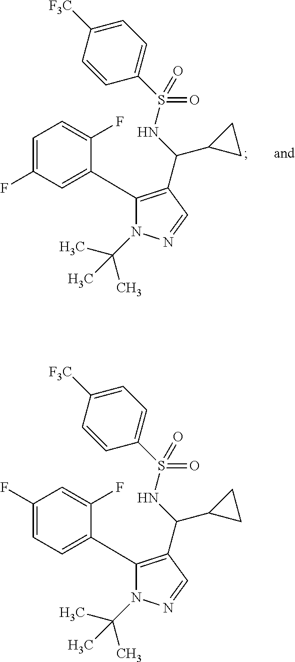

or a salt or solvate thereof.

### Base

The base used in the cyclization of compounds of Formula (III) to compounds of Formula (IV) (method 2) can be any base. Exemplary bases include salts of organic and inorganic anions, such as carbonates, phosphates, acetates and the like. In a particular example, the base is a carbonate, such as potassium carbonate (K₂CO₃), sodium carbonate (Na₂CO₃), cesium carbonate (Cs₂CO₃), or a phosphate, such as potassium phosphate (K₂PO₄) or sodium phosphate (Na₂PO₄). In one example, the base is Cs₂CO₃. In another example, the base is other than acetate. In another example, the base is other than cesium acetate (CsOAc). The base can be present in the reaction mixture in any amount. In one example, the base is used in an amount equivalent to between about 1 equivalent (100 mol %) and about 10 equivalents relative to the molecule of Formula (III). In another example, the base is used in an amount equivalent to between about 1.5 equivalents (150 mol %) and about 5 equivalents relative to the first molecule of Formula (III). In yet another example, the base is used in an amount equivalent to between about 1.5 equivalents and about 3.0 equivalents relative to the first molecule of Formula (III).

### Solvent, Reaction Temperature and Reaction Time

In one example, the compound of Formula (III) is contacted with the base in the presence of a solvent. The solvent can be any solvent. The term “solvent” includes mixtures of at least two different solvents. Exemplary solvents, which are useful in the above method, include DMF, DMA, DMSO, aromatic solvents, such as xylene (i.e., ortho-, meta- and para-xylene), toluene and mixtures thereof. In one example, the solvent has a boiling point of at least about 100° C., at least about 120° C. or at least about 130° C. The reaction mixture can optionally be pressurized enabling the use of a greater variety of solvents with lower boiling points. The reaction mixture is typically heated to a temperature between about 100° C. and about 150° C. In one example, the reaction mixture is heated to between about 110° C. and 140° C. In another example, the reaction mixture is heated to about 120 to about 130° C. In one example, the solvent used in the above method is DMA. In another example, the solvent used in the above method is DMF. In another example, the solvent used in the above method is DMA and the reaction mixture is heated under nitrogen to between about 100 and about 130° C.

In one example, the reaction mixture is heated for a period between about 1 hour (h) and about 100 h. In another example, the reaction mixture is heated for a period between about 1 h and about 50 h. In yet another example, the reaction mixture is heated for a period between about 1 h and about 40 h, between about 1 h and about 30 h or between about 1 h and about 20 h. In a further example, the reaction mixture is heated for a period between about 1 h and about 15 h, between about 1 h and about 12 h or between 1 h and about 10 h. In one example, the reaction mixture is heated to between about 100° C. and about 150° C. for a period between about 2 h and about 12 h.

### Reaction Yield

In one example, the second compound of Formula (IV) is formed from the first molecule of Formula (III) with a reaction yield (e.g., isolated yield, mol/mol) between about 50% and about 100%, between about 60% and about 100%, between about 70% and about 100%, between about 80% and about 100% or between about 90% and about 100% (mol/mol) relative to the amount of starting material (first molecule of Formula (III)) used in the reaction. In another example, the second molecule of Formula (IV) is formed with a reaction yield of at least about 80%, at least about 90% or at least about 95%. In yet another example, the second molecule of Formula (IV) is formed with a reaction yield of at least about 96%, at least about 97%, at least about 98% or at least about 99% (mol/mol) relative to the first molecule of Formula (III). Reaction yields can alternatively be determined using various chromatography methods (e.g., LC, LC/MS). The ratio between the amount of starting material (first molecule) and the amount of product (second molecule) in the final reaction mixture can be determined, for example, using “area under the curve” (AUC) values.

The additional processing steps that are discussed in reference to method 1 (e.g., purification, isolation, crystallization, etc.) may also be employed with this method. In another example, method 2 further includes: removing the amino protecting group R³ from the second molecule of Formula (IV), thereby forming a third molecule having a structure according to Formula (B):

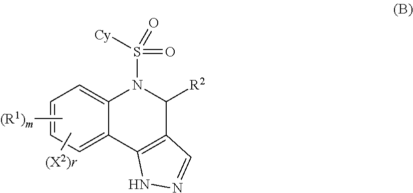

or a salt, solvate, tautomer, mixture of tautomers, stereoisomer or mixture of stereoisomers thereof, wherein Cy, m, r, X², R¹ and R² are defined as hereinabove, e.g., for Formula (I) and Formula (IV).

The above method of cyclizing compounds of Formula (III) can further include processing steps relating to the making of compounds of Formula (III) as outlined hereinbelow (methods 3-5).

### Synthesis of Compounds of Formula (I) and Formula (III)

The invention further provides methods of making compounds of Formula (I) and Formula (III).

### Method 3

An exemplary method includes: (i) contacting a first compound having a structure according to Formula (X):

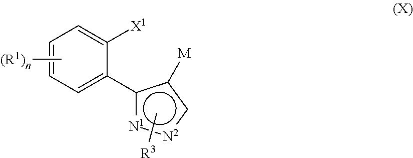

wherein M is Li (lithium) or MgX, wherein X is halogen (e.g., Cl, Br, or I); and N¹, N², X¹, n, R¹ and R³ are defined as in Formula (I) above,
with a sulfinylimine having a structure according to Formula (XI):

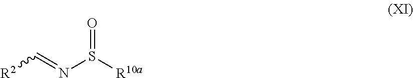

wherein R² is defined as in Formula (I) above,
thereby forming a second compound having a structure according to Formula (XII):

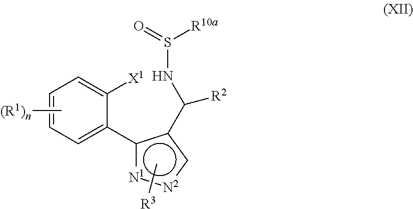

or a salt or solvate thereof, wherein X¹, n, R¹, R², and R³ are defined as herein above.

In Formula (XI) and Formula (XII), R^{10a} is selected from alkyl (e.g., C₁-C₈-alkyl), alkenyl (e.g., C₁-C₈-alkenyl), alkynyl (e.g., C₁-C₈-alkynyl), haloalkyl (e.g., C₁-C₆-haloalkyl), cycloalkyl (e.g., C₃-C₆-cycloalkyl), heteroalkyl (e.g., 2- to 6- membered heteroalkyl), heterocycloalkyl (e.g., 3- to 8-membered heterocycloalkyl), aryl (e.g., phenyl) and heteroaryl (e.g., 5- or 6-membered heteroaryl), each of which is optionally substituted with from 1 to 5 substituents selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴, R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷_{C(S)NR}¹⁴R¹⁵, NR¹⁷S(O)₂R¹⁶, S(O)₂NR¹⁴R¹⁵, S(O)R¹⁶ and S(O)₂R¹⁶, wherein R¹⁴, R¹⁵R¹⁶ and R¹⁷ are as defined as in Formula (I). In one example, in Formula (XI) and Formula (XII), R^{10a} is branched (C₃-C₈-alkyl) (e.g., iso-propyl, iso-butyl or tert-butyl), branched 3- to 8-membered heteroalkyl, cycloalkyl (e.g., C₃-C₁₀-cycloalkyl), 3- to 6-membered heterocycloalkyl, aryl, and 5- or 6-membered heteroaryl. In another example, R^{10a} is tert-butyl.

In one example, in Formula (X) and Formula (XII), R³ is covalently bonded to N¹ of the pyrazole ring. In another example, in Formula (X) and Formula (XII), R³ is covalently bonded to N² of the pyrazole.

It is also contemplated that in Formula (X), the phenyl ring that carries X¹ and R¹ can be replaced with a 6-membered heteroaromatic ring comprising from 1 to 3 nitrogen atoms. Exemplary heteroaromatic rings include pyridine and pyrimidine.

In one example, the compound of Formula (X) has a structure according to Formula (Xa), Formula (Xb), Formula (Xc) or Formula (Xd):

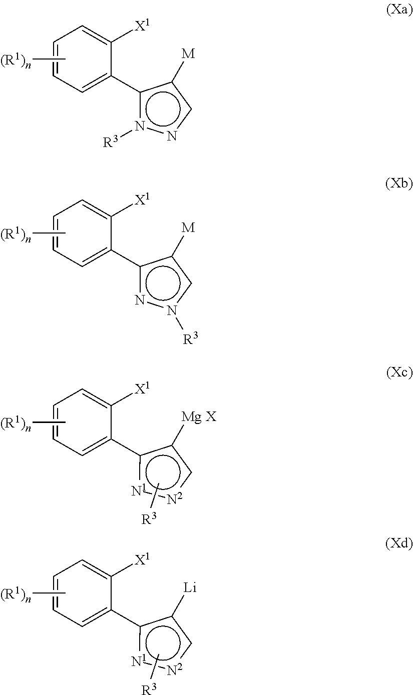

wherein N¹, N², X¹, n, R¹ and R³ are defined as for Formula (I), Mg is magnesium, Li is lithium and X is halogen. In one example, in Formula (Xc), X is Cl or Br or I.

In another example, the compound of Formula (X) has a structure according to Formula (Xe), Formula (Xf), Formula (Xg), Formula (Xh) or Formula (Xi):

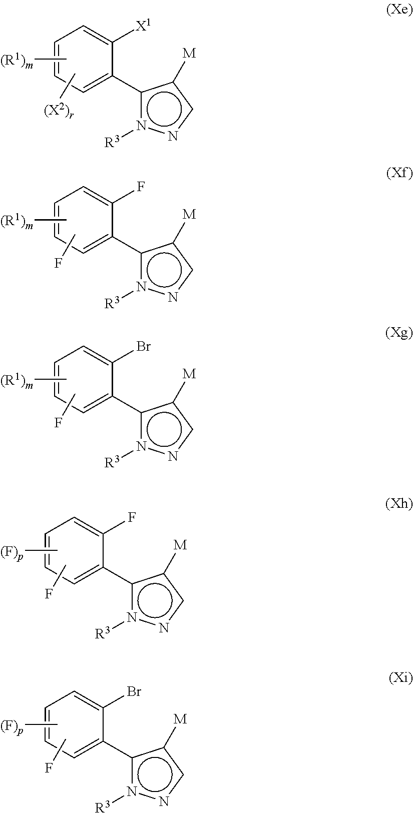

wherein M, X¹, X², r, p, m, R¹ and R³ are as defined herein, e.g., for Formula (I), Formula (IIIa), (IIIb) and Formula (X), respectively. In one example, in Formula (Xh) or Formula (Xi), p is 0 or 1. In another example in the above formulae, R³ is tert-butyl.

In another example, the compound of Formula (X) has a structure according to Formula (Xj), Formula (Xk), Formula (Xl), or Formula (Xm):

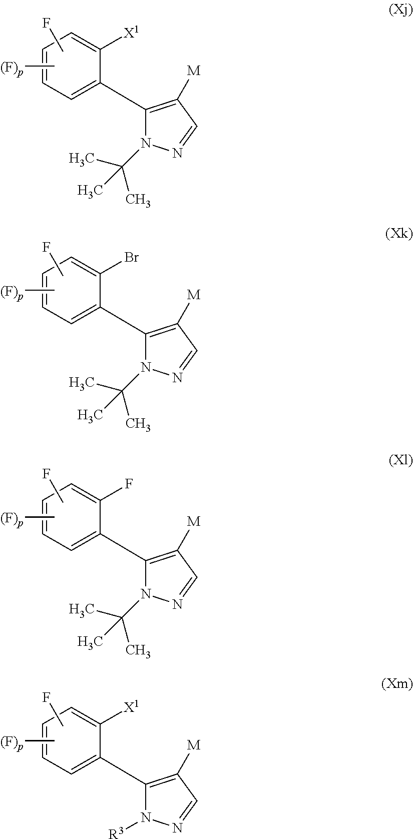

wherein X¹ is defined as above; p is selected from 0 to 3, M is Li or MgX, wherein X is Cl, Br or I.

In another example, the compound of Formula (X) has a structure according to one of the following formulae:

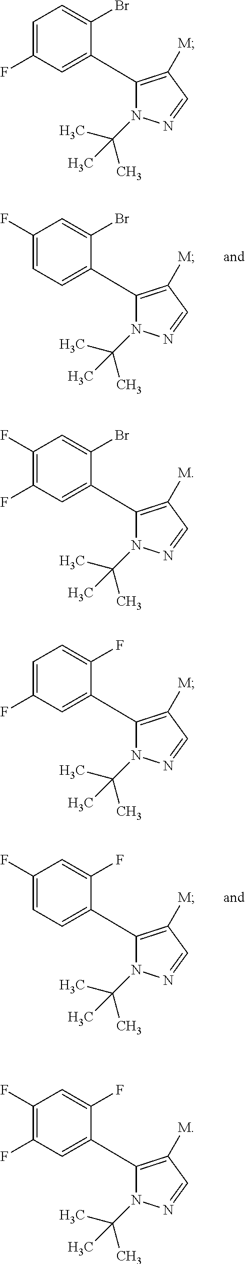

wherein M is Li or MgX, wherein X is Cl, Br or I.

Method 3 can further include:
(iii) removing a sulfinyl moiety from the second compound of Formula (XII), thereby forming a third compound (amine) having a structure according to Formula (XIII):

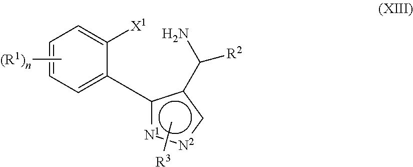

or a salt, solvate, stereoisomer or mixture of stereoisomers thereof, wherein N¹, N², X¹, n, R¹, R² and R³ are defined as herein above. In one example, the sulfinyl moiety is removed using acid, such as HCl.

The method can further include:
(iv) contacting the third compound of Formula (XIII) with a sulfonylchloride having the formula:

Cy-S(O)₂Cl

wherein Cy is as defined as in Formula (I), thereby forming a compound having a structure is defined herein according to Formula (I) or Formula (III).

### Stereoselectivity

In one embodiment, when using the above method (method 3), compounds of Formula (I) or Formula (III) are formed with improved stereoselectivity (with respect to the stereocenter involving R²) when compared to using known methods (see, e.g., US2008/0021056). Stereoselectivity is improved through the use of a chiral sulfinylimine, such as:

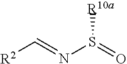

wherein R² and R^{10a} are defined herein. The stereoinducing effect can be enhanced using branched (bulky) residues, such as tert-butyl, or cycloalkyl as R^{10a}.

In one example, the compound of Formula (I), (III), (XII), or (XIII) is formed with a stereoselectivity (e.g., R versus S configuration at the stereocenter involving R²) of at least about 8:1, at least about 9:1 or at least about 10:1 (as determined, e.g., using a chiral chromatography column; AUC/AUC). In another example, the compound of Formula (I) or (III) is formed with a stereoselectivity of at least about 12:1, at least about 14:1, at least about 16:1, at least about 18:1, or at least about 20:1. In yet another example, the compound of Formula (I) or (III) is formed with a stereoselectivity of at least about 22:1, at least about 24:1, at least about 26:1, at least about 28:1, or at least about 30:1. In a further example, the compound of Formula (I) or (III) is formed with a stereoselectivity of at least about 32:1, at least about 34:1, at least about 36:1, at least about 38:1, or at least about 40:1. In one examples, the compound of formula (I), (III), (XII), or (XIII) is formed with a stereoselectivity of at least about 14:1, favoring the R configuration at the stereocenter involving the R².

The above method can further include one or more of the following steps:
(v) cyclizing the compound of Formula (I) or Formula (III) as described hereinabove (see, e.g., method 1 or method 2), thereby forming a compound of Formula (II) or (IV);
(vi) purifying the compound of Formula (II) or (IV), e.g., as described herein above (e.g., method 1 or method 2);
(vii) removing the amino protecting group R³, thereby forming a compound having a structure according to Formula (A) or Formula (B), e.g., as described herein above (e.g., method 1 or method 2); and
(viii) purifying the resulting de-protected analog, e.g., as described hereinabove (e.g., method 1 or method 2).

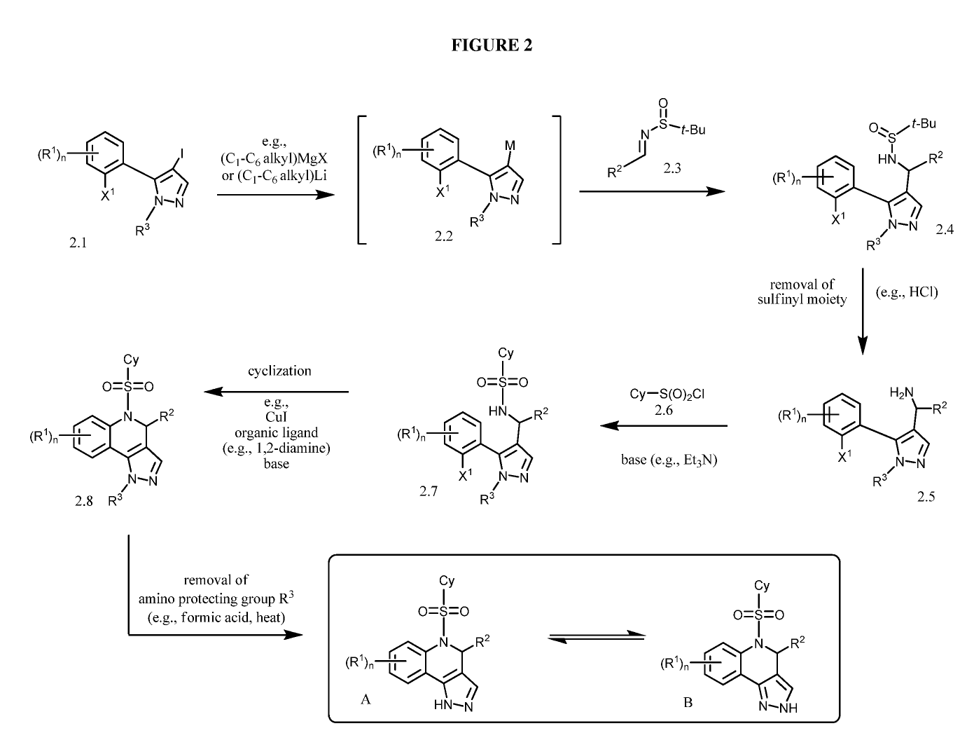

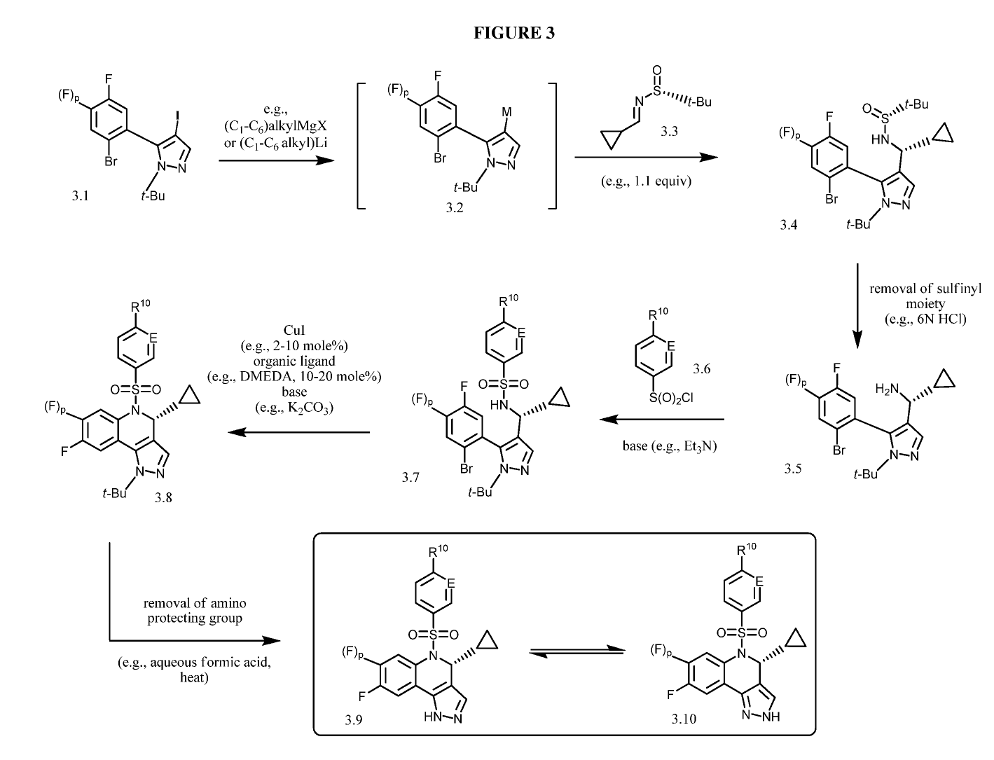

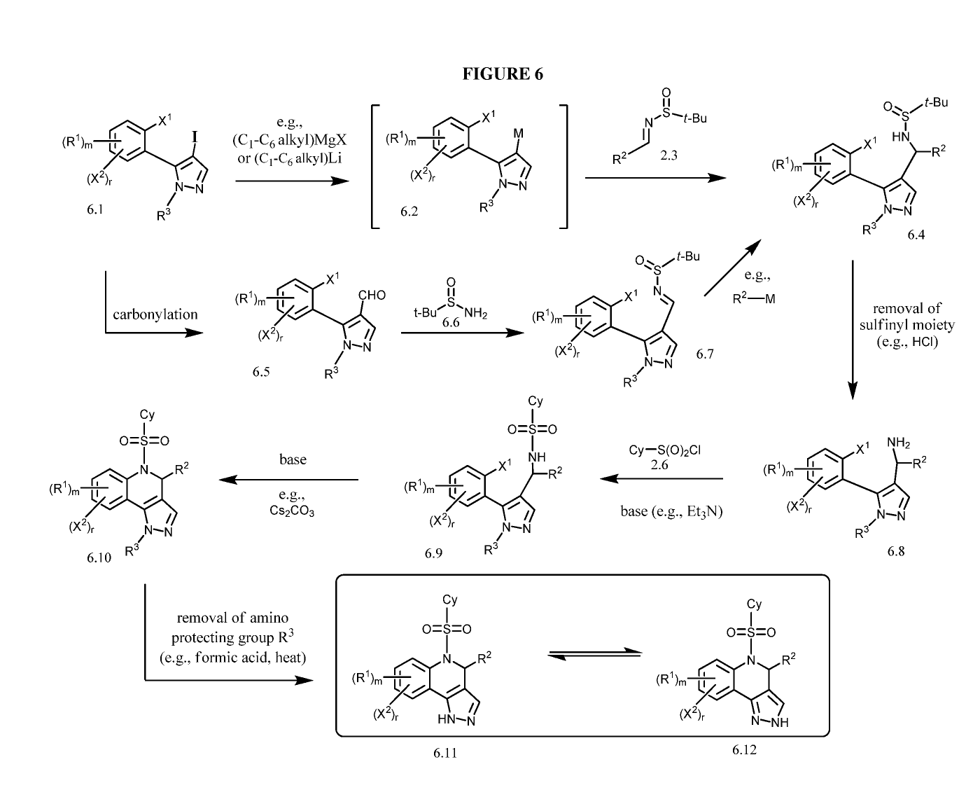

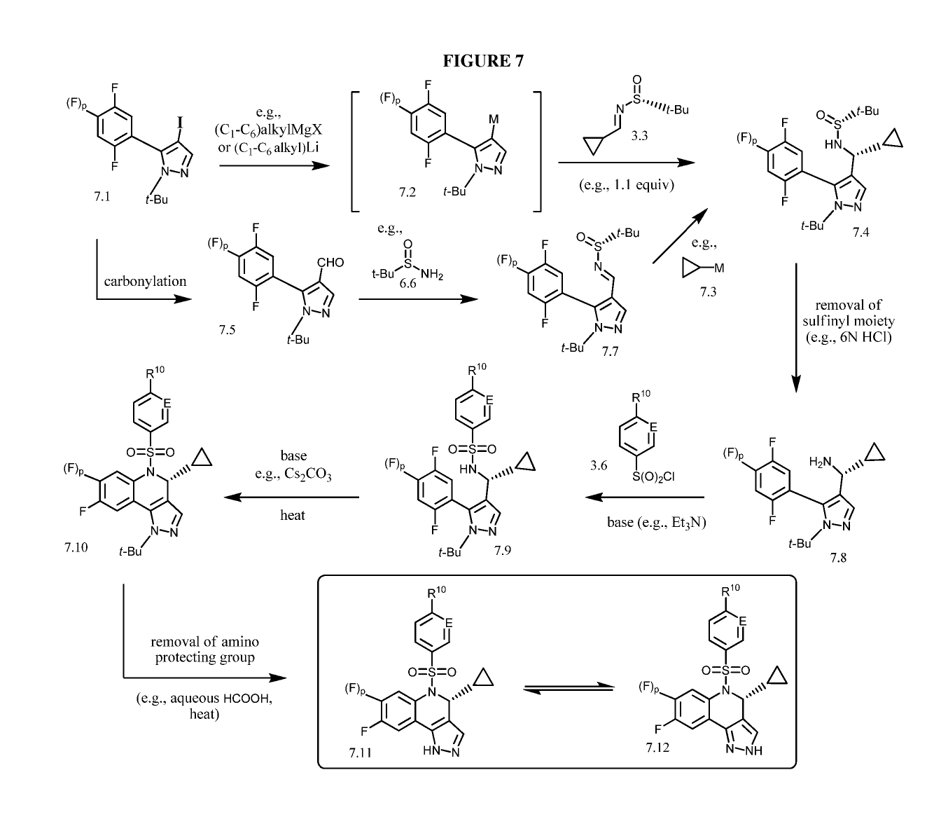

Overall reaction yields, e.g., starting from compounds of Formula (X) and ending with compounds of Formula (II) are significantly improved resulting in more-cost efficient processes. Improved overall reaction yields are partly due to more efficient cyclization procedure used to convert compounds of Formula (I) to compounds of Formula (II) (method 1) or compounds of Formula (III) to compounds of Formula (IV) (method 2), in which the formation of impurities, such as de-brominated side products are significantly reduced. Further, in one embodiment, the current process of converting compounds of Formula (X) to compounds of Formula (I) or (III) (see, e.g., FIGS. 2 and 3, and FIGS. 6 and 7) does not require isolation of intermediate products (e.g., conventional work-up and/or purification). Hence, these processes require a reduced amount of organic solvent for workup and chromatography. In one example, compounds of Formula (XII), compounds of Formula (XIII), and compounds of Formula (I) or Formula (III) are not isolated prior to subsequent reaction steps. For example, compounds of Formula (II) or (IV) can be synthesized from compounds of Formula (X) in a one-pot reaction sequence (e.g., merely involving solvent swapping between reaction steps).

### Method 3a

In one example, the current disclosure provides a process that includes: (i) contacting a first compound having a structure according to Formula (Xm):

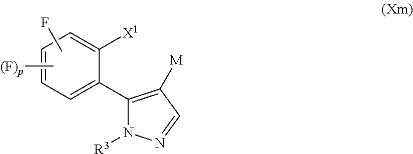

wherein X¹ is F, Cl or Br; p is 0 or 1; M is Li or MgX, wherein X is Cl, Br or I; and R³ is an amino protecting group as defined herein,
with a sulfinylimine having a structure according to Formula (XIa):

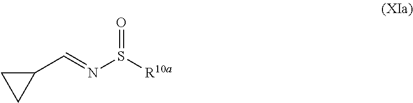

wherein R^{10a} is branched (C₃-C₈-alkyl) (e.g., iso-propyl, iso-butyl or tert-butyl), branched 3- to 8-membered heteroalkyl, cycloalkyl (e.g., C₃-C₁₀-cycloalkyl), 3- to 6-membered heterocycloalkyl, aryl, and 5- or 6-membered heteroaryl, thereby forming a second compound having a structure according to Formula (XIIa):

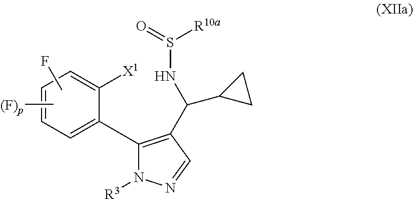

or a salt or solvate thereof, wherein p, X¹, R³ and R^{10a} are defined as above for Formula (Xm) and (Xla), respectively.

The method can further include: (ii) removing a sulfinyl moiety from the second compound of Formula (XIIa), thereby forming a third compound having a structure according to Formula (XIIIa):

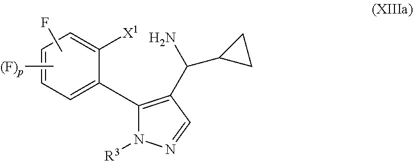

or a salt or solvate, thereof, wherein p is 0 or 1; X¹ is F, Cl or Br; and R³ is defined as for Formula (Xm), above. The removing of the sulfinyl moiety can be accomplished using acid, such as aqueous HCl.

The method can further include: (iii) contacting the third compound of Formula (XIIIa) with a sulfonylchloride having the formula:

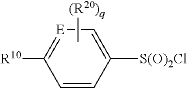

wherein E is N or CH; R¹⁰ is defined as for Formula (Ic) herein above; q is an integer selected from 0 to 3; and R²⁰ is defined below,
thereby forming a fourth compound having a structure according to Formula (C):

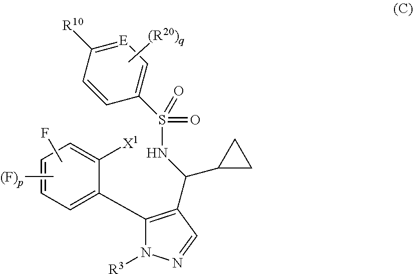

or a salt or solvate thereof, wherein p is 0 or 1; E is CH or N; and R³ and R¹⁰ are defined as above. In one example in Formula (C), R¹⁰ is selected from halogen, CN, C₁-C₃-alkyl (e.g., methyl), and C₁-C₃-halo alkyl (e.g., CF₃).

In Formula (C), q is an integer selected from 0 to 3. In one example, q is selected from 0 and 1. In another example q is 0. In yet another example, q is 1. In Formula (C), R²⁰ is selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶, wherein R¹⁴, R¹⁵, R¹⁶ and R¹⁷ are defined herein, e.g., for Formula (I). In one example, q is 1 and R²⁰ is selected from (C₁-C₃)alkyl, (C₁-C₃)haloalkyl, halogen, and OR¹⁴. In one example, q is 1 and R²⁰ is —OR¹⁴. In another example, q is 1 and R²⁰ is selected from OH and (C₁-C₃)alkoxy (e.g., methoxy).

The method can further include cyclizing the fourth compound of Formula (C) according to a method described herein above (e.g., in method 1 or method 2). For example, the method can further include (iv) contacting the fourth compound of Formula (C) with a catalyst including copper (e.g., a copper ion) and at least one organic ligand (e.g., 1,2-diamine), under reaction conditions sufficient to form a fifth compound having a structure according to Formula (D):

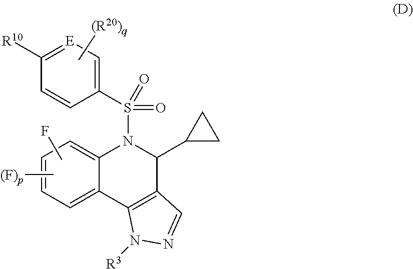

or a salt or solvate thereof, wherein p is 0 or 1; E is CH or N; and q, R³, R¹⁰, and R²⁰ are defined as above. Suitable copper reagents, organic ligands and reaction conditions are described herein above for method 1.

In one example in Formula (Xm), Formula (XIIa), Formula (XIIIa), Formula (C), and Formula (D), R³ is tert-butyl.

The method can further include (v) purifying the compound of Formula (D), e.g., as described herein above in method 1.

The method can further include (vi) removing the amino protecting group from the compound of Formula (D), e.g., as described herein above for the formation of compounds of Formula (A), thereby forming a compound having a structure according to Formula (E):

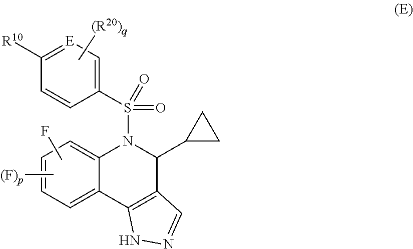

or a salt or solvate thereof, wherein p is 0 or 1; E is CH or N; and q, R¹⁰ and R²⁰ are defined as above.

In one example in Formula (Xm), Formula (XIIa), Formula (XIIIa), Formula (C), Formula (D), and Formula (E), R¹⁰ is haloalkyl (e.g., CF₃). In another example in Formula (Xm), Formula (XIIa), Formula (XIIIa), Formula (C), Formula (D), and Formula (E), E is CH. In yet another example in Formula (Xm), Formula (XIIa), Formula (XIIIa), Formula (C), Formula (D), and Formula (E), E is N. In a further example in Formula (Xm), Formula (XIIa), Formula (XIIIa), Formula (C), Formula (D), and Formula (E), p is 1. In a further example in Formula (Xm), Formula (XIIa), Formula (XIIIa), Formula (C), Formula (D), and Formula (E), p is 0. In yet another example in Formula (Xm), Formula (XIIa), Formula (XIIIa), Formula (C), Formula (D), and Formula (E), q is 0. In yet another example in Formula (Xm), Formula (XIIa), Formula (XIIIa), Formula (C), Formula (D), and Formula (E), q is 1 and R²⁰ is alkoxy (e.g., methoxy). In yet another example in Formula (Xm), Formula (XIIa), Formula (XIIIa), Formula (C), Formula (D), and Formula (E), R¹⁰ is CF₃, E is N and p is 0. In yet another example in Formula (Xm), Formula (XIIa), Formula (XIIIa), Formula (C), Formula (D), and Formula (E), R¹⁰ is CF₃, E is CH and p is 1.

The method can further include: (vii) purifying the de-protected analog of Formula (E), e.g., as described herein above in method 1.

Exemplary methods according to the above embodiments of method 1, method 2, method 3 and method 3a are outlined in FIGS. 1, 2 3, 6 and 7, respectively.

In one example in the above method, the compound of Formula (XIIa), the compound of Formula (XIIIa), and the compound of Formula (C) are not isolated prior to subsequent reaction steps. For example, compounds of Formula (C) can be synthesized from compounds of Formula (X) in a one-pot reaction sequence.

### Synthesis of Compounds of Formula (X)

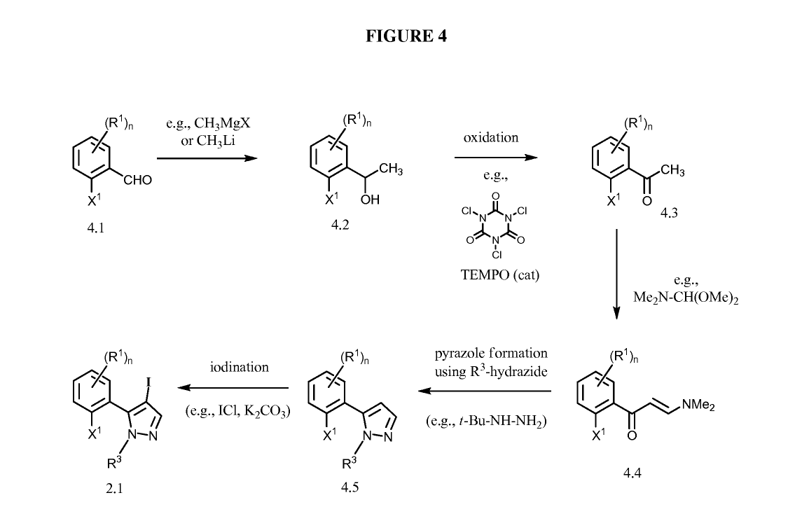

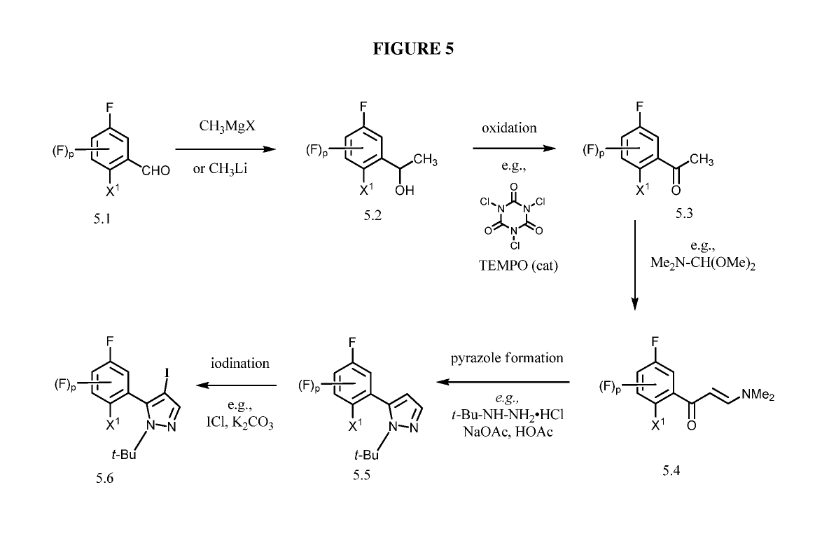

The above described methods can further include processing steps relating to the making of compounds of Formula (X). Compounds of Formula (X) can be synthesized, e.g., using methods outlined herein, e.g., those depicted in FIGS. 4 and 5.

### Method 4

In one example, the compound of Formula (X) is synthesized from a corresponding acetophenone. An exemplary method includes: (i) contacting a compound having structure according to Formula (XXX):

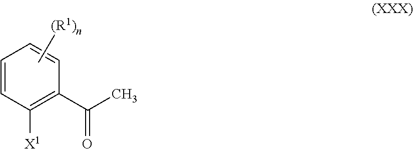

or a salt or solvate thereof, wherein X¹, n and R¹ are defined as herein above for Formula (I),
with a 1,1-dialkoxy-N,N-dialkylmethanamine (e.g., 1,1-dimethoxy-N,N-dimethylmethanamine, also known as dimethylformamide-dimethylacetal or DMF-DMA),
under reaction conditions sufficient to form a compound having a structure according to Formula (XXXI):

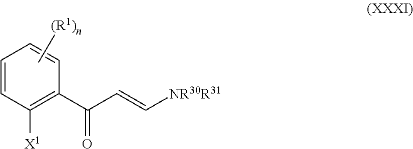

or a salt or solvate thereof, wherein X¹, n and R¹ are defined as herein above, e.g., for Formula (I); and R³⁰ and R³¹ are independently selected from C₁-C₄-alkyl. In on example, R³⁰ and R³¹ are both methyl.

It is also contemplated that in Formula (XXX) and Formula (XXXI), the phenyl ring that carries X¹ and R¹ can be replaced with a 6-membered heteroaromatic ring comprising from 1 to 3 nitrogen atoms. Exemplary heteroaromatic rings include pyridine and pyrimidine.

The above method can further include:
(ii) contacting the compound of Formula (XXXI) with a mono-substituted hydrazine having the formula:

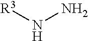

or a salt thereof (e.g., HCl salt), wherein R³ is defined herein, e.g., for Formula (I), under reaction conditions sufficient to form a pyrazole having a structure according to Formula (XXXII):

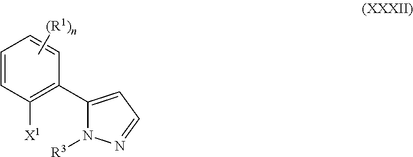

or a salt or solvate thereof, wherein X¹, n, R¹ and R³ are defined as herein above, e.g., for Formula (I).

The above method can further include:
(iii) contacting the pyrazole of Formula (XXXII) with a halogenation reagent under reaction conditions sufficient to form a compound having a structure according to Formula (XXXIII):

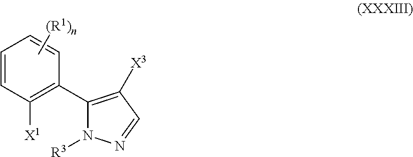

or a salt or solvate thereof, wherein X¹, n, R¹ and R³ are defined as herein above, e.g., for Formula (I); and X³ is halogen (e.g., Br, Cl or I). In one example in Formula (XXXIII), X³ is iodine (I). In another example in Formula (XXXIII), X³ is Br. In yet another example in Formula (XXXIII), X³ is Cl. In one example, X³ is I and the halogenation (iodination) reagent is selected from iodine monochloride (ICl), optionally in combination with a base (e.g., a basic salt, e.g., carbonate, such as K₂CO₃), and iodide (e.g., NaI or KI), optionally in combination with an oxidizing reagent, such as Oxone®.

The above method can further include: (iii) contacting the halo pyrazole of Formula (XXXIII) with an alkyl-magnesium halide (e.g., alkyl-MgCl), Li, or an organolithium reagent (e.g., tert-BuLi, n-BuLi) under reaction conditions sufficient to produce an activated intermediate of Formula (X).

In one example, according to any of the above embodiments, the leaving group X¹ is a member selected from Br, Cl, F, mesylate and tosylate. In another example, according to any of the above embodiments, X¹ is Br. In another example, according to any of the above embodiments, X¹ is F. In another example, according to any of the above embodiments, n is 1 or 2 and each R¹ is F.

### Method 4a

Another exemplary method of this disclosure includes: (i) contacting a compound having structure according to Formula (XXXa):

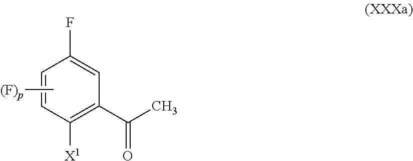

or a salt or solvate thereof, wherein X¹ is F, Cl or Br, and p is an integer selected from 0 and 1,
with a 1,1-dialkoxy-N,N-dialkylmethanamine (e.g., 1,1-dimethoxy-N,N-dimethylmethanamine, DMF-DMA),
under reaction conditions sufficient to form a compound having a structure according to Formula (XXXIa):

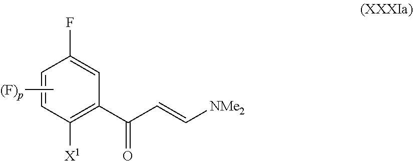

or a salt or solvate thereof, wherein X¹ is F, Cl or Br, and p is an integer selected from 0 and 1. In one example in Formula (XXXa) and (XXXIa), X¹ is Br. In another example in Formula (XXXa) and (XXXIa), X¹ is F.

The above method can further include:
(ii) contacting the compound of Formula (XXXIa) with a mono-substituted hydrazine having the formula:

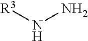

or a salt thereof (e.g., HCl salt), wherein R³ is defined herein, e.g., for Formula (I),
under reaction conditions sufficient to form a pyrazole having a structure according to Formula (XXXIIa):

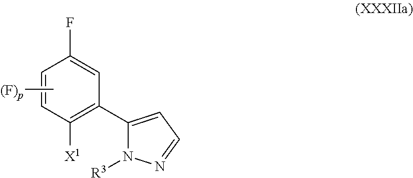

or a salt or solvate thereof, wherein X¹ is F, Cl or Br, and p is an integer selected from 0 and 1. In one example in Formula (XXXIIa), R³ is tert-butyl. In another example in Formula (XXXIIa), X¹ is Br. In yet another example in Formula (XXXIIa), X¹ is F.

The above method can further include:
(iii) contacting the pyrazole of Formula (XXXIIa) with an iodination reagent under reaction conditions sufficient to form an iodopyrazole having a structure according to Formula (XXXIIIa):

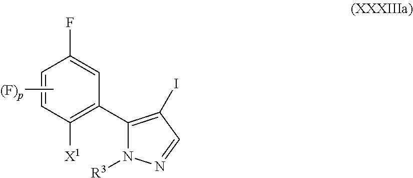

or a salt or solvate thereof, wherein X¹ is F, Cl or Br, p is 0 or 1, and R³ is defined herein, e.g., for Formula (I). In one example, the iodination reagent is selected from iodine monochloride (ICl), optionally in combination with a base (e.g., a basic salt, e.g., carbonate, such as K₂CO₃), and iodide (e.g., NaI or KI), optionally in combination with an oxidizing reagent, such as Oxone®.

The above method can further include:
(iii) contacting the iodo pyrazole of Formula (XXXIIIa) with an alkyl-magnesium halide (e.g., alkyl-MgCl), Li, or an organolithium reagent (e.g., tert-BuLi, n-BuLi) to produce an activated intermediate having the formula:

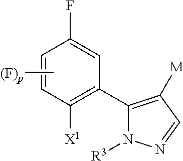

wherein M, X¹, p and R³ are defined as above.

The above methods (method 4 and 4a) are particularly useful for the regio-selective preparation of pyrazoles of Formula (XXXII) and (XXXIIa), and halo- (e.g., iodo-) pyrazoles of Formula (XXXIII) or (XXXIIIa), in which R³ is covalently attached to N¹ of the pyrazole ring, a significant improvement over known methods of producing substituted pyrazoles, in which regioisomers are formed, which have to be separated (see, e.g., US2008/0021056, e.g., page 82).

Acetophenone analogs of Formula (XXX) and Formula (XXXa) (or related molecules), which can be used as starting materials in the above described methods (e.g., method 4 and 4a), can be made using art recognized methods or those described herein, e.g., Examples 1 and 2.

### Method 5

In one example, the acetophenone of formula (XXX) is prepared using a method comprising:
(i) contacting a benzaldehyde having a structure according to Formula (XXXIV):

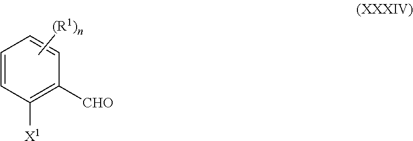

or a salt or solvate thereof, wherein X¹, R¹ and n are defined as herein above, e.g., for Formula (I),
with a methyl-magnesium halide (e.g., CH₃MgX, wherein X is halogen, such as Cl, Br or I) or CH₃Li to produce a compound having a structure according to Formula (XXXV):

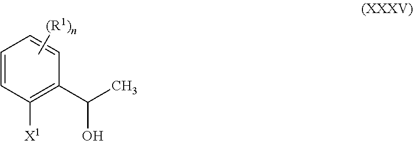

or a salt or solvate thereof, wherein X¹, R¹ and n are defined as herein above, e.g., for Formula (I).

It is also contemplated that in Formula (XXXIV) and Formula (XXXV), the phenyl ring that carries X¹ and R¹ can be replaced with a 6-membered heteroaromatic ring comprising from 1 to 3 nitrogen atoms. Exemplary heteroaromatic rings include pyridine and pyrimidine.

The above method can further include: (ii) contacting the hydroxyethyl derivative of Formula (XXXV) with an oxidizing reagent, under reaction conditions sufficient to form an acetophenone having a structure according to formula (XXX). Oxidizing reagents, which are useful for the oxidation of a secondary hydroxyl group to an oxo (keto) group are known to those of skill in the art. In one example, the oxidizing reagent is trichloroiso-cyanuric acid in combination with a catalyst, such as TEMPO (2,2,6,6-tetramethylpiperidine 1-oxyl).

### Method 5a

In another example, the acetophenone of formula (XXXa) is prepared using a method comprising:
(i) contacting a benzaldehyde having a structure according to Formula (XXXIVa):

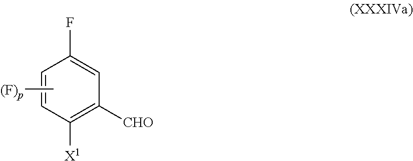

or a salt or solvate thereof, wherein X¹ and p are defined as herein above,
with a methyl-magnesium halide (e.g., CH₃MgX, wherein X is halogen, such as Cl, Br or I) or CH₃Li to produce a compound having a structure according to Formula (XXXVa):

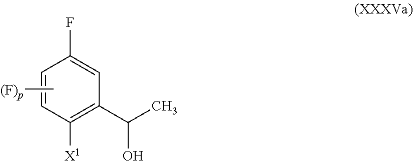

or a salt or solvate thereof, wherein X¹ and p are defined as herein above.

The above method can further include: (ii) contacting the hydroxyethyl derivative of Formula (XXXVa) with an oxidizing reagent, under reaction conditions sufficient to form an acetophenone having a structure according to formula (XXXa). Oxidizing reagents, which are useful for the oxidation of a secondary hydroxyl group to an oxo (keto) group are known to those of skill in the art. In one example, the oxidizing reagent is trichloroiso-cyanuric acid in combination with a catalyst, such as TEMPO (2,2,6,6-tetramethylpiperidine 1-oxyl).

In another example, the acetophenone of formula (XXX) is prepared using the method outlined in Scheme 3, below:

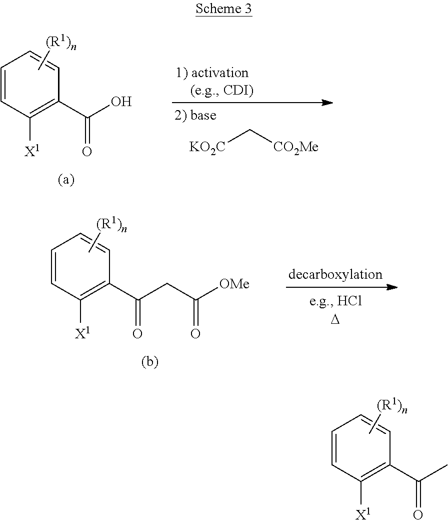

In Scheme 3, the benzoic acid derivative (a) is first treated with an activation reagent, such as carbonyldiimidazole (CDI), thereby forming an activated carboxylic acid derivative. The activated intermediate is further reacted with 3-methoxy-3-oxopropanoate. Subsequent decarboxylation leads to the methyl 3-oxo-3-phenylpropanoate (b). Further decarboxylation, initiated e.g., with acid (e.g., HCl) and heat, leads to the acetophenone.

Exemplary methods according to the above embodiments of methods 4, 4a, 5 and 5a are outlined in FIGS. 4 and 5, respectively.

Other exemplary methods of this disclosure are combinations of the above described methods. For example, method 6 comprises method 3, followed by method 1. Method 7 comprises method 3, followed by method 2. Method 8 comprises method 3a, followed by method 1. Method 9 comprises method 3a, followed by method 2. Method 10 comprises method 4, followed by method 3, followed by method 1. Method 11 comprises method 4, followed by method 3, followed by method 2. Method 12 comprises method 4a, followed by method 3a, followed by method 1. Method 13 comprises method 4a, followed by method 3a, followed by method 2. Each of these methods may be preceded by method 5 or 5a, respectively.

### III. Compositions

In another aspect, the invention further provides molecules, which are useful, e.g., as intermediates in the methods and processes described in this disclosure.

In one example, the invention provides a compound having a structure according to Formula (XX):

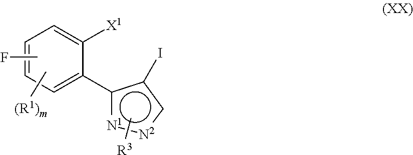

or a salt or solvate thereof, wherein N¹ and N² are nitrogen atoms of a pyrazole ring; I is iodine; m is an integer selected from 0 to 3; X¹ is halogen (e.g., I, Br, Cl or F); and R¹ and R³ are defined as for Formula (I). In one example, X¹ in Formula (XX) is Br. In another example, X¹ in Formula (XX) is F.

In Formula (XX), R³ is an amino protecting group covalently bonded to either N¹ or N² of the pyrazole ring. In one example in Formula (XX), R³ is selected from alkyl (e.g., C₁-C₁₀-alkyl), alkenyl (e.g., C₁-C₁₀-alkenyl), alkynyl (e.g., C₁-C₁₀-alkynyl), haloalkyl (e.g., C₁-C₁₀-haloalkyl), cycloalkyl (e.g., C₃-C₁₀-cycloalkyl), heterocycloalkyl (e.g., 3- to 10-membered heterocycloalkyl), aryl (e.g., phenyl), and heteroaryl (e.g., 5- or 6-membered heteroaryl), each optionally substituted with from 1 to 5 (e.g., from 1 to 3) substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷_{C(S)NR}¹⁴R¹⁵, NR¹⁷S(O)₂R¹⁶, S(O)₂NR¹⁴R¹⁵, S(O)R¹⁶ and S(O)₂R¹⁶. In another example, R³ in Formula (XX) is selected from optionally substituted C₁-C₆ alkyl, optionally substituted C₁-C₆ alkenyl, and optionally substituted C₁-C₆ alkynyl. In yet another example, R³ in Formula (XX) is C₁-C₁₀-alkyl (e.g., tert-butyl) or aryl(C₁-C₃)alkyl (e.g., benzyl). In another example, R³ in Formula (XX) is tert-butyl. In yet another example, R³ in Formula (XX) is 2-(trimethylsilyl)ethoxymethyl (SEM ether) or methoxymethyl (MOM ether).

In one example in Formula (XX), R³ is covalently bonded to N¹ of the pyrazole ring. In another example in Formula (XX), R³ is covalently bonded to N² of the pyrazole ring. In another example, R³ in Formula (XX) is tert-butyl and is covalently bonded to N¹ of the pyrazole ring.

In one example, each R¹ in Formula (XX) is independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶, and S(O)₂R⁶, wherein the alkyl, alkenyl, alkynyl, heteroalkyl, cycloalkyl, heterocycloalkyl, aryl and heteroaryl is optionally substituted, e.g., with from 1 to 5 (e.g., from 1 to 3) substituents independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶, wherein R¹⁴, R¹⁵, R¹⁶ and R¹⁷ are defined as for Formula (I), above. R⁴, R⁵, R⁶ and R⁷ are also defined as for Formula (I), above. In one example, in Formula (XX), each R¹ is independently selected from optionally substituted C₁-C₃ alkyl (e.g., methyl, ethyl or propyl), halogen (e.g., F, Cl or Br) and CN. In another example, m is 1 and R¹ is halogen. In yet another example, m is 1 and R¹ is F. In a further example, m is 0 (and R¹ is absent). In one example, R¹ is selected from halogen, C₁-C₄ alkyl, C₁-C₄ haloalkyl, or C₁-C₄ haloalkoxy.

In one example of Formula (XX), the pyrazole ring is not substituted with C₁-C₂ alkyl (e.g., methyl). In another example in Formula (XX), the R³ is not the following group

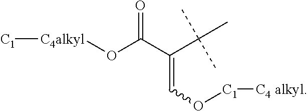

In one example, X¹ in Formula (XX) is Br and the compound has a structure according to Formula (XXIa):

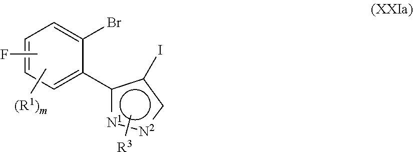

wherein N¹, N², m, R¹ and R³ are defined as for Formula (XX) above.

In another example, X¹ in Formula (XX) is F and the compound has a structure according to Formula (XXIb):

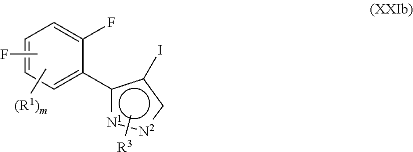

wherein N¹, N², m, R¹ and R³ are defined as for Formula (XX) above.

In yet another example, the compound of Formula (XX) has a structure according to Formula (XXIc) or Formula (XXId):

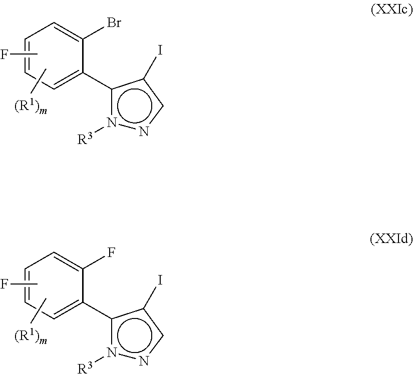

wherein m, R¹ and R³ are defined as for Formula (XX), above.

In a further example, m in Formula (XXIc) or Formula (XXId) is 0 or 1, and R¹ (when present) is F. Exemplary compounds include:

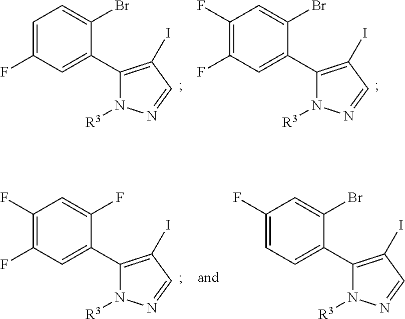

or a salt or solvate thereof, wherein R³ is defined as for Formula (XX) above. In one example, in the above structures, R³ is (C₁-C₆)alkyl (e.g., tert-butyl) or benzyl.

The invention further provides a compound having a structure according to Formula (XXII):

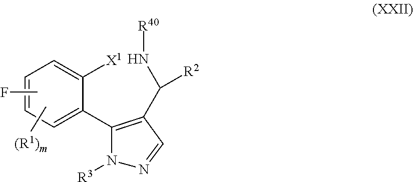

or a salt or solvate thereof, wherein m, X¹, R¹ and R³ are defined as for Formula (XX) above. In one example, in Formula (XXII), X¹ is F. In another example, in Formula (XXII), X¹ is Br.

In one example in Formula (XX), (XXIa), (XXIb), (XXIc), (XXId), and (XXII), R³ is a silyl ether, such as 2-(trimethylsilyl)ethoxymethyl (SEM) ether; or an alkoxymethyl ether, such as methoxymethyl (MOM) ether, tert-butoxymethyl (BUM) ether, benzyloxymethyl (BOM) ether, or methoxyethoxymethyl (MEM) ether. In one example in Formula (XX), (XXIa), (XXIb), (XXIc), (XXId), and (XXII), R³ is selected from C₁-C₁₀-alkyl (e.g., tert-butyl) and benzyl. In another example in Formula (XX), (XXIa), (XXIb), (XXIc), (XXId), and (XXII), R³ is other than OH or alkoxy. In yet another example, R³ in Formula (XX), (XXIa), (XXIb), (XXIc), (XXId), and (XXII) is other than SEM ether (i.e., —CH₂OCH₂CH₂—SiMe₃).

In one example of Formula (XX), (XXIa), (XXIb), (XXIc), (XXId), and (XXII), the pyrazole ring is not substituted with C₁-C₂ alkyl (e.g., methyl). In another example in Formula (XX), (XXIa), (XXIb), (XXIc), (XXId), and (XXII), the R³ is not the following group:

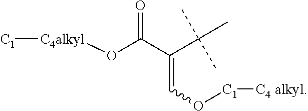

In Formula (XXII), R² is selected from H, alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), each optionally substituted with from 1 to 5 (e.g., from 1 to 3) substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In one example, R² is selected from C₁-C₄-alkyl, C₃-C₆ cycloalkyl, and aryl, all of which are optionally substituted. In one example, R² in Formula (XXII) is optionally substituted (C₃-C₆)-cycloalkyl. In another example, R² in Formula (XXII) is optionally substituted cyclopropyl. In yet another example, R² in Formula (XXII) is cyclopropyl. In one example, R² in Formula (XXII) is other than COOR¹⁴ (e.g., other than COOH). In another example, R² in Formula (XXII) is other than carboxyl-substituted C₁-C₃-alkyl (i.e., —CH₂COOH).

In one example, in Formula (XXII), R⁴⁰ is selected from H, S(O)R^{10a} and S(O)₂Cy, wherein R^{10a} is defined as for Formula (XI), and Cy is defined as for Formula (I). In another example in Formula (XXII), R⁴⁰ is H. In yet another example in Formula (XXII), R⁴⁰ is S(O)R^{10a}, wherein R^{10a} is defined as for Formula (XI). In a further example in Formula (XXII), R⁴⁰ is S(O)₂Cy, wherein Cy is defined as for Formula (I).

In another example in Formula (XXII), R⁴⁰ is selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), each optionally substituted with from 1 to 5 (e.g., from 1 to 3) substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In one example, R⁴⁰ is optionally substituted C₃-C₆-alkyl.

In a further example, R⁴⁰ in Formula (XXII) is S(O)R^{10a}, wherein R^{10a} is selected from alkyl (e.g., C₁-C₈-alkyl), alkenyl (e.g., C₁-C₈-alkenyl), alkynyl (e.g., C₁-C₈-alkynyl), haloalkyl (e.g., C₁-C₆-haloalkyl), cycloalkyl (e.g., C₃-C₆-cycloalkyl), heterocycloalkyl (e.g., 3- to 8-membered heterocycloalkyl), aryl (e.g., phenyl) and heteroaryl (e.g., 5- or 6-membered heteroaryl), each optionally substituted with from 1 to 5 substituents selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶. In another example in Formula (XXII), R⁴⁰ is S(O)R^{10a}, wherein R^{10a} is branched (C₃-C₈-alkyl) (e.g., iso-propyl, iso-butyl or tert-butyl), branched 3- to 8-membered heteroalkyl, cycloalkyl (e.g., C₃-C₁₀-cycloalkyl), 3- to 6-membered heterocycloalkyl, aryl, and 5- or 6-membered heteroaryl. In another example in Formula (XXII), R⁴⁰ is S(O)R^{10a}, wherein R^{10a} is tert-butyl.

In yet another example in Formula (XXII), R⁴⁰ is S(O)₂Cy, wherein Cy is selected from cycloalkyl (e.g., C₃-C₆-cycloalkyl), heterocycloalkyl (e.g., 3- to 8-membered heterocycloalkyl), aryl (e.g., phenyl) and heteroaryl (e.g., 5- or 6-membered heteroaryl), wherein the cycloalkyl, heterocycloalkyl, aryl or heteroaryl is optionally substituted with from 1 to 5 substituents, wherein each substituent is independently selected from alkyl (e.g., C₁-C₆-alkyl), alkenyl (e.g., C₁-C₆-alkenyl), alkynyl (e.g., C₁-C₆-alkynyl), haloalkyl (e.g., C₁-C₆-haloalkyl), heteroalkyl (e.g., 2- to 6-membered heteroalkyl), cycloalkyl (e.g., C₃-C₆-cycloalkyl), heterocycloalkyl (e.g., 3- to 8-membered heterocycloalkyl), aryl (e.g., phenyl), heteroaryl (e.g., 5- or 6-membered heteroaryl), CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶.

In a further example in Formula (XXII), R⁴⁰ is S(O)₂Cy, wherein Cy is selected from aryl (e.g., phenyl), and 5- or 6-membered heteroaryl, wherein the aryl or heteroaryl is optionally substituted with from 1 to 3 substituents selected from C₁-C₃-alkyl, C₁-C₃-alkenyl, C₁-C₃-alkynyl, C₁-C₃-haloalkyl, halogen, CN, OH and methoxy. In one example, Cy is aryl or heteroaryl, each of which is optionally substituted with halogen, C₁-C₄ haloalkyl, or C₁-C₄ haloalkoxy. In one example, R⁴⁰ in Formula (XXII) is S(O)₂Cy, wherein Cy is optionally substituted phenyl. In another example R⁴⁰ in Formula (XXII) is S(O)₂Cy, wherein Cy is optionally substituted pyridyl. In yet another example, R⁴⁰ in Formula (XXII) is S(O)₂Cy, wherein Cy is haloalkyl-substituted phenyl. In a further example, R⁴⁰ in Formula (XXII) is S(O)₂Cy, wherein Cy is haloalkyl-substituted pyridyl. In yet another example, R⁴⁰ in Formula (XXII) is S(O)₂Cy, wherein Cy is CF₃-substituted phenyl or CF₃-substituted pyridyl. In another example, Cy is phenyl or pyridyl, wherein the phenyl or pyridyl is optionally substituted with 1 to 4 substituents selected from halogen, C₁-C₄ haloalkyl (e.g., -CF₃), or C₁-C₄ haloalkoxy (e.g., —OCF₃).

In Formula (XXII) and its embodiments, each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring. Each R¹⁶ is selected from acyl, C₁-C₆-alkyl, C₁-C₆ haloalkyl,C₁-C₆-alkenyl, C₁-C₆-alkynyl, 2- to 6-membered heteroalkyl, aryl, 5- or 6-membered heteroaryl, C₃-C₈ cycloalkyl, and 3- to 8-membered heterocycloalkyl.

In one example, in Formula (XXII), X¹ is Br and the compound has a structure according to Formula (XXIIa):

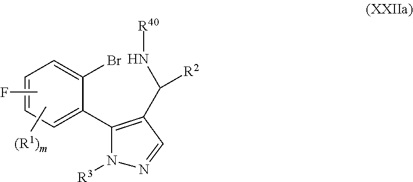

or a salt or solvate thereof, wherein m, R¹, R², R³ and R⁴⁰ are defined as for Formula (XXII) hereinabove.

In another example, in Formula (XXII), X is F and the compound has a structure according to Formula (XXIIb):

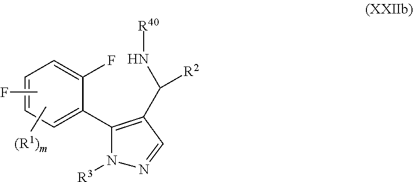

or a salt or solvate thereof, wherein m, R¹, R², R³ and R⁴⁰ are defined as for Formula (XXII) hereinabove.

In another example, the compound of Formula (XXII) has a structure selected from:

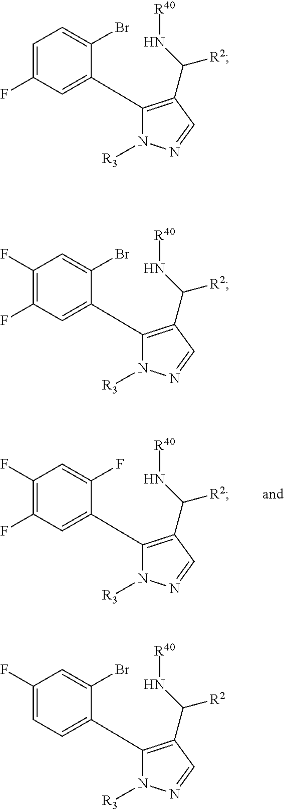

or a salt or solvate thereof, wherein R², R³ and R⁴⁰ are defined as for Formula (XXII) hereinabove.

In one example, according to any of the above embodiments of Formula (XXII), (XXIIa) and (XXIIb), R² is cyclopropyl. In another example, according to any of the above embodiments of Formula (XXII), (XXIIa) and (XXIIb), R⁴⁰ is H. In yet another example, according to any of the above embodiments of Formula (XXII), (XXIIa) and (XXIIb), R⁴⁰ is S(O)₂Cy, wherein Cy is defined as herein above. In a further example, according to any of the above embodiments of Formula (XXII), (XXIIa) and (XXIIb), R⁴⁰ is S(O)₂Cy, wherein Cy is trifluoromethyl-substituted phenylsulfonyl, e.g., 4-(trifluoromethyl)phenylsulfonyl; or trifluoromethyl-substituted pyridylsulfonyl, e.g., 6-(trifluoromethyl)pyridin-3-ylsulfonyl.

The invention further provides a compound selected from:
5-(2-bromo-5-fluorophenyl)-1-tert-butyl-4-iodo-1H-pyrazole;
5-(2-bromo-4-fluorophenyl)-1-tert-butyl-4-iodo-1H-pyrazole;
5-(2-bromo-4,5-difluorophenyl)-1-tert-butyl-4-iodo-1H-pyrazole; and
1-tert-butyl-4-iodo-5-(2,4,5-trifluorophenyl)-1H-pyrazole, or a salt or solvate thereof.

The invention further provides a compound selected from:
(5-(2-bromo-5-fluorophenyl)-1-tert-butyl-1H-pyrazol-4-yl)(cyclopropyl)methanamine;
(5-(2-bromo-4-fluorophenyl)-1-tert-butyl-1H-pyrazol-4-yl)(cyclopropyl)methanamine;
(5-(2-bromo-4,5-difluorophenyl)-1-tert-butyl-1H-pyrazol-4-yl)(cyclopropyl)-methanamine;
(1-tert-butyl-5-(2,4,5-trifluorophenyl)-1H-pyrazol-4-yl)(cyclopropyl)methanamine;
   (1R)-(5-(2-bromo-5-fluorophenyl)-1-tert-butyl-1H-pyrazol-4-yl)(cyclopropyl)methanamine;
(1R)-(5-(2-bromo-4-fluorophenyl)-1-tert-butyl-1H-pyrazol-4-yl)(cyclopropyl)methanamine;
(1R)-(5-(2-bromo-4,5-difluorophenyl)-1-tert-butyl-1H-pyrazol-4-yl)(cyclopropyl)methanamine; and
(1R)-(1-tert-butyl-5-(2,4,5-trifluorophenyl)-1H-pyrazol-4-yl)(cyclopropyl)methanamine, or a salt or solvate thereof.

The invention further provides a compound selected from:
N-((5-(2-bromo-5-fluorophenyl)-1-tert-butyl-1H-pyrazol-4-yl)(cyclopropyl)methyl)-2-methylpropane-2-sulfinamide;
N-((1R)-(5-(2-bromo-5-fluorophenyl)-1-tert-butyl-1H-pyrazol-4-yl)(cyclopropyl)methyl)-2-methylpropane-2-sulfinamide;
N-((5-(2-bromo-4,5-difluorophenyl)-1-tert-butyl-1H-pyrazol-4-yl)(cyclopropyl)methyl)-2-methylpropane-2-sulfinamide;
N-((1R)-(5-(2-bromo-4,5-difluorophenyl)-1-tert-butyl-1H-pyrazol-4-yl)(cyclopropyl)methyl)-2-methylpropane-2-sulfinamide;
N-((5-(2-bromo-4-fluorophenyl)-1-tert-butyl-1H-pyrazol-4-yl)(cyclopropyl)methyl)-2-methylpropane-2-sulfinamide;
N-((1R)-(5-(2-bromo-4-fluorophenyl)-1-tert-butyl-1H-pyrazol-4-yl)(cyclopropyl)methyl)-2-methylpropane-2-sulfinamide;
N-((1-tert-butyl-5-(2,4,5-trifluorophenyl)-1H-pyrazol-4-yl)(cyclopropyl)methyl)-2-methylpropane-2-sulfinamide; and
N-((1R)-(1-tert-butyl-5-(2,4,5-trifluorophenyl)-1H-pyrazol-4-yl)(cyclopropyl)methyl)-2-methylpropane-2-sulfinamide, or a salt or solvate thereof.

The invention further provides a compound selected from:
N-((5-(2-bromo-4-fluorophenyl)-1-tert-butyl-1H-pyrazol-4-yl)(cyclopropyl)methyl)-4-(trifluoromethyl)benzenesulfonamide;
N-((1R)-(5-(2-bromo-4-fluorophenyl)-1-tert-butyl-1H-pyrazol-4-yl)(cyclopropyl)-methyl)-4-(trifluoromethyl)benzenesulfonamide;
N-((5-(2-bromo-4-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4-fluorophenyl)-1-tert-butyl-1H-pyrazol-4-yl)(cyclopropyl)methyl)-6-(trifluoromethyl)pyridine-3-sulfonamide;
N-((5-(2-bromo-4,5-difluorophenyl)-1-tert-butyl-1H-pyrazol-4-yl)(cyclopropyl)methyl)-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-4-(trifluoromethyl)benzenesulfonamide;
N-((5-(2-bromo-4,5-difluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4,5-difluorophenyl)-1-tert-butyl-1H-pyrazol-4-yl)(cyclopropyl)methyl)-6-(trifluoromethyl)pyridine-3-sulfonamide;
N-((5-(2-bromo-5-fluorophenyl)-1-tert-butyl-1H-pyrazol-4-yl)(cyclopropyl)methyl)-4-(trifluoromethyl)benzenesulfonamide;
N-((1R)-(5-(2-bromo-5-fluorophenyl)-1-tert-butyl-1H-pyrazol-4-yl)(cyclopropyl)methyl)-4-(trifluoromethyl)benzenesulfonamide;
N-((5-(2-bromo-5-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide;
N-((1-tert-butyl-5-(2,4,5-trifluorophenyl)-1H-pyrazol-4-yl)(cyclopropyl)methyl)-4-(trifluoromethyl)benzenesulfonamide;
N-((1R)-(1-tert-butyl-5-(2,4,5-trifluorophenyl)-1H-pyrazol-4-yl)(cyclopropyl)methyl)-4-(trifluoromethyl)benzenesulfonamide;
N-((1-tert-butyl-5-(2,4,5-trifluorophenyl)-1H-pyrazol-4-yl)(cyclopropyl)methyl)-6-(trifluoromethyl)pyridine-3-sulfonamide;
N-((1R)-(1-tert-butyl-5-(2,4,5-trifluorophenyl)-1H-pyrazol-4-yl)(cyclopropyl)methyl)-6-(trifluoromethyl)pyridine-3-sulfonamide;
N-((1R)-(1-tert-butyl-5-(2,4,5-trifluorophenyl)-1H-pyrazol-4-yl)(cyclopropyl)methyl)-3-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-5-fluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-3-methoxy-4-(trifluoromethyl)benzenesulfonamide;
N-((1R)-(1-tert-butyl-5-(2,4,5-trifluorophenyl)-1H-pyrazol-4-yl)(cyclopropyl)methyl)-2-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-2-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-2-methoxy-4-(trifluoromethyl)benzenesulfonamide; and
N-((1R)-(5-(2-bromo-5-fluorophenyl)-1-tert-butyl-1H-pyrazol-4-yl)(cyclopropyl)methyl)-2-methoxy-4-(trifluoromethyl)benzenesulfonamide, or a salt or solvate thereof.

The invention further provides a compound selected from:
(R)-1-tert-butyl-4-cyclopropyl-7,8-difluoro-5-(2-methoxy-4-(trifluoromethyl)phenylsulfonyl)-4,5-dihydro-1H-pyrazolo[4,3-c]quinoline; and
(R)-1-tert-butyl-4-cyclopropyl-7,8-difluoro-5-(3-methoxy-4-(trifluoromethyl)phenylsulfonyl)-4,5-dihydro-1H-pyrazolo[4,3-c]quinoline.

The methods and compositions of this invention are exemplified by the following examples, which should not be construed as limiting the scope of this disclosure. Analogous structures and alternative synthetic routes are within the scope of the invention will be apparent to those of ordinary skill in the art.

### Examples

### General

The starting materials and various intermediates described herein may be obtained from commercial sources, prepared from commercially available compounds, and/or prepared using known synthetic methods. For example, certain intermediates, can be synthesized by known processes using either solution- or solid phase techniques as shown below. Representative procedures for preparing the compounds of this disclosure are outlined herein.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups (e.g., amino protecting groups) are described in T. W. Greene and P. G. M. Wuts, Protecting Groups in Organic Synthesis, Third Edition, Wiley, New York, 1999, and references cited therein.

Reagents and solvents obtained from commercial suppliers were used without further purification unless otherwise stated. Thin layer chromatography was performed on precoated 0.25 mm silica gel plates (E. Merck, silica gel 60, F₂₅₄). Visualization was achieved using UV illumination or staining with phosphomolybdic acid, ninhydrin or other common staining reagents. Flash chromatography was performed using either a Biotage Flash 40 system and prepacked silica gel columns or hand packed columns (E. Merck silica gel 60, 230-400 mesh). Preparatory HPLC was performed on a Varian Prepstar high performance liquid chromatograph. ¹H NMR spectra were recorded on either a Varian Gemini 300 MHz spectrometer or a Bruker Avance 300 MHz spectrometer unless otherwise indicated. Chemical shifts are reported in ppm (δ) and were calibrated using the undeuterated solvent resonance as internal standard. Mass spectra were recorded on an Agilent series 1100 mass spectrometer connected to an Agilent series 1100 HPLC. Unless otherwise stated, all temperatures are in degrees Celsius (° C.).

In the examples below, the following abbreviations have the following meanings. If an abbreviation is not defined, it has its generally accepted meaning.
μm=micrometer or micron
AcOH=acetic acid
AUC=area under the curve (referring to a chromatogram)
BINAP=2,2′-bis(diphenylphosphino)-1,1′-binaphthyl
br s=broad singlet
CDI=carbonyldiimidazole
CsOAc=cesium acetate
d=doublet
dba=dibenzylidene acetone
DBU=1,8-diazabicyclo[5.4.0]undec-7-ene
DCE=1,2-dichloroethane
DCM=dichloromethane
dd=doublet of doublets
ddd=doublet of doublet of doublets
DEAD=diethyl azodicarboxylate
DESA=N,N-diethylsalicylamide
DMAP=4-dimethylaminopyridine
DME=1,2-dimethoxyethane
DMEDA or DMED=N,N′-dimethylethylenediamine
DMF=dimethylformamide
DMF-DMA=N,N-dimethylformamide dimethyl acetal
DMSO=dimethylsulfoxide
DPPA=diphenylphosphoryl azide
DSC=differential scanning calorimetry
EDTA=ethylenediamine tetraacetic acid
eq=equivalent
Et=ethyl
Et₃N=triethyl amine
EtOAc=ethyl acetate
EtOH=ethanol
g=gram
GC=gas chromatography
h=hour
HEPES=4-(2-hydroxyethyl)-1-piperazineethanesulfonic acid
HOAc=Acetic acid
HPLC=high performance liquid chromatography
Hz=Hertz
in=inch
iPr=isopropyl
i-PrOH=iso-propanol
kg=kilogram
L=liter
LC/MS=liquid chromatography mass spectroscopy
LCMS=liquid chromatography mass spectroscopy
M=molar
m=multiplet
Me=methyl
MeOH=methanol
min=minute
mL=milliliter
mm=millimeter
mmol=millimole
mol=mole
MTBE=methyl tert-butylether
N=normal
NaOAc=sodium acetate
PBS=phosphate buffered saline
ppm=parts per million
Pr=propyl
q=quartet
R_{f}=retention factor (ratio of distance traveled by substance/distance traveled by solvent front)
Rₜ=retention time
RT=room temperature
SEM=2-(trimethylsilyl)ethoxymethyl
TFA=trifluoroacetic acid
THF=tetrahydrofuran
TLC=thin layer chromatography
uL=microliter
Xantphos=4,5-bis(diphenylphosphino)-9,9-dimethylxanthene

### Example 1

### Synthesis of 5-(2-bromo-5-fluorophenyl)-1-tert-butyl-4-iodo-1H-pyrazole

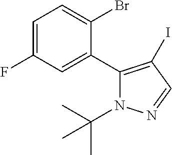

1.1. Preparation of 1-(2-bromo-5-fluorophenyl)ethanol

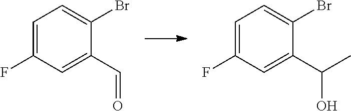

To a solution of 2-bromo-5-fluorobenzaldehyde (49.4 g, 243 mmol) in dry THF (500 mL) under N₂ at −78° C. was added methyl magnesium chloride (3.0 M in THF, 89 mL, 268 mmol) dropwise over a period of about 1 hour (h), such that the internal temperature was maintained below −65° C. After complete addition of the Grignard reagent, the reaction mixture was allowed to warm to room temperature. At 0° C. the reaction was quenched by dropwise addition of 1 M HCl (250 mL) over a period of about 30 minutes (min), during which time the internal temperature was maintained below 10° C. A further portion of 1 M HCl (100 mL) was then added to solubilize residual magnesium salts. After separation of the organic phase, the aqueous phase was extracted with MTBE. The combined organic phases were washed with 1 M HCl, H₂O, and brine, dried over Na₂SO₄, and concentrated in vacuo to give 1-(2-bromo-5-fluorophenyl)ethanol as a yellow oil (53.4 g, 100%) in 95% purity (HPLC). This material was used without further purification in the next reaction step. ¹H-NMR (300 MHz, CDCl₃) δ 7.47 (dd, J=8.7, 5.2 Hz, 1H), 7.35 (dd, J=9.7, 3.0 Hz, 1H), 6.87 (ddd, J=8.7, 7.8, 3.1 Hz, 1H), 5.19 (q, J=6.4 Hz, 1H), 2.05 (br s, 1H), 1.48 (d, J=6.4 Hz, 3H).

1.2. Preparation of 1-(2-bromo-5-fluorophenyl)ethanone

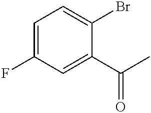

To a solution of 1-(2-bromo-5-fluorophenyl)ethanol (53.4 g, 0.2438 moles) in dichloromethane (500 mL) at 0.2° C. was added trichloroiso-cyanuric acid (59.5 g, 0.256 moles, 1.05 eq). To the resulting suspension was added TEMPO (2,2,6,6-tetramethylpiperidine 1-oxyl; 188 mg, 1.20 mmol, 0.5 mol %). The reaction mixture was allowed to stir at ice bath temperature until the oxidation was complete (HPLC, about 4.5 h). The resulting reaction mixture was diluted with MTBE (about 1300 mL) and washed with 1 N NaOH (2×250 mL), 1 N HCl containing potassium iodide (to remove TEMPO; 8 g KI in 1000 mL 1 N HCl; 2×250 mL), 1 N NaHCO₃ containing sodium thiosulfate (to remove I₂; 15 g Na₂S₂O₃ in 1000 mL 1 N NaHCO₃), 1 N HCl containing potassium iodide (1×200 mL), 1 N NaHCO₃ containing sodium thiosulfate (2×200 mL), and brine (150 mL). After drying (anhydrous MgSO₄), the solvent was removed at reduced pressure to give 1-(2-bromo-5-fluorophenyl)ethanone as a pale amber liquid (52.96g, about 97%) in 98% purity by HPLC, which was used in the next step without further purification. ¹H-NMR (300 MHz, CDCl₃) δ 7.59 (dd, J=8.7, 4.9 Hz, 1H), 7.19 (dd, J=8.5, 3.0 Hz, 1H), 7.04 (ddd, J=9.1, 7.8, 3.0 Hz, 1H), 2.64 (s, 3H).

1.3. Preparation of 1-(2-Bromo-5-fluorophenyl)-3-(dimethylamino)prop-2-en-1-one

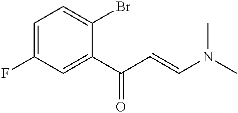

A solution of 1-(2-bromo-5-fluorophenyl)ethanone (15.7 g, 72.2 mmol) in dimethylformamide dimethyl acetal (24.0 mL, 181 mmol) was heated to 80° C. for 3 h. The reaction mixture was allowed to cool to room temperature and was then cooled to about 0° C. Water (100 mL) was slowly added to the reaction mixture while the internal temperature was maintained below about 20° C. The resulting biphasic mixture was further diluted with MTBE and water. The aqueous phase was extracted with MTBE, and the combined organic phases were washed with water and brine, dried over Na₂SO₄, and concentrated in vacuo to give 1-(2-bromo-5-fluorophenyl)-3-(dimethylamino)prop-2-en-1-one as a dark orange solid (19.6 g, 100%). This material was used without further purification in the next reaction step. ¹H-NMR (500 MHz, CDCl₃) δ 7.51 (d, J=8.8, 4.9 Hz, 1H), 7.02-7.12 (m, 2H), 6.93 (dt, J=8.2, 3.0 Hz, 1H), 5.28 (d, J=12.5 Hz, 1H), 3.12 (br s, 3H), 2.89 (s, 3H). Alternatively, a standard aqueous workup was employed to isolate the title compound as a brown oil, which crystallized upon standing (250 g, 96% yield, 97% purity by HPLC).

1.4. Preparation of 5-(2-Bromo-5-fluorophenyl)-1-tert-butyl-1H-pyrazole (4)

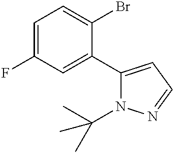

To a solution of 1-(2-bromo-5-fluorophenyl)-3-(dimethylamino)prop-2-en-1-one (19.6 g, 72.0 mmol) in AcOH (75 mL) were added NaOAc (14.8 g, 180 mmol) and tert-butyl-NHNH₂.HCl (17.9 g, 144 mmol). The reaction mixture was heated to 60° C. for 5 h and was then allowed to cool to room temperature. At about 0° C., a 50% NaOH solution (about 50-75 mL) was added dropwise until a pH of about 12 was reached, maintaining the internal temperature below 25° C. The reaction mixture was then extracted with MTBE. The combined organic phases were washed with H₂O (until the pH of the aqueous phase was below 8), 1 M HCl, H₂O, and brine, dried over Na₂SO₄, and concentrated in vacuo to give 5-(2-bromo-5-fluorophenyl)-1-tert-butyl-1H-pyrazole as a brown oil (20.4 g, 95% over two steps). This material was used in the next step without further purification. ¹H NMR (500 MHz, CDCl₃) δ 7.59 (dd, J=8.8, 5.2 Hz, 1H), 7.54 (d, J=1.8 Hz, 1H), 7.11 (dd, J=8.8, 3.0 Hz, 1H), 7.03 (ddd, J=8.5, 7.9, 3.0 Hz, 1H), 6.13 (d, J=1.8 Hz, 1H), 1.50 (s, 9H). On a larger (300 g) scale the yield was about 98% and the product was obtained in 89% purity by HPLC, which solidified upon standing at room temperature.

1.5. Preparation of 5-(2-Bromo-5-fluorophenyl)-1-tert-butyl-4-iodo-1H-pyrazole

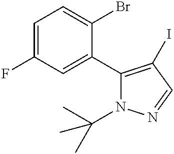

To a solution of 5-(2-bromo-5-fluorophenyl)-1-tert-butyl-1H-pyrazole (18.79 g, 63.2 mmol) in 2-propanol (400 mL) and H₂O (100 mL) was added KI (11.5 g, 69.6 mmol) and Oxone® (42.8 g, 69.6 mmol) and the reaction mixture was stirred at room temperature for 6 h, during which time the product precipitated. It was diluted with H₂O (1.5 L) to dissolve inorganic salts, and the solid product was collected by filtration and allowed to air dry to afford 24.3 g (91%) of a tan solid. Re-crystallization from 2-propanol (about 2.5 mL/g) gave 5-(2-bromo-5-fluorophenyl)-1-tert-butyl-4-iodo-1H-pyrazole (21.8 g, 81%) as an off-white crystalline solid (DSC 134° C.).

Iodination was found to be equally effective with iodine monochloride (ICI) in dichloromethane containing potassium carbonate. The latter conditions were more suitable for large-scale production because the KI/Oxone® procedure generated a large quantity of poorly soluble inorganic salts, complicating the workup.

To a mixture of 5-(2-bromo-5-fluorophenyl)-1-tert-butyl-1H-pyrazole (161.1 g, 0.542 mol), dichloromethane (2.0 L), and K₂CO₃ (1.36 mol, 187.3 g) at 15° C. was added a solution of iodine monochloride (1.08 mol, 176.0 g) in dichloromethane (0.5 L) in one portion. The reaction mixture was stirred at room temperature for 2 h. The contents of the reactor were cooled to 15° C. and 0.5 M Na₂S₂O₃ solution (1 L) was added. The resulting biphasic mixture was stirred vigorously for 1 h (loss of color was observed), and the layers were separated. The organic phase was washed with 0.5 M Na₂S₂O₃ (1 L), H₂O (1 L), and brine (1 L), and was then dried over Na₂SO₄. Concentration in vacuo provided 221.5 g of a tan solid. Re-crystallization from 2-propanol (about 575 mL) gave 5-(2-bromo-5-fluorophenyl)-1-tert-butyl-4-iodo-1H-pyrazole (189.9 g, 83%) as an off-white solid. ¹H NMR (500 MHz, CDCl₃) δ 7.66 (dd, J=8.8, 5.2 Hz, 1H), 7.60 (s, 1H), 7.09 (dt, J=8.5, 3.0 Hz, 1H), 7.03 (dd, J=8.4, 2.9 Hz, 1H), 1.48 (s, 9H).

The above described process for the synthesis of 5-(2-bromo-5-fluorophenyl)-1-tert-butyl-4-iodo-1H-pyrazole from 1-(2-bromo-5-fluorophenyl)ethanone proceeded with an overall yield of about 78%.

### Example 2

### Alternate synthesis of 5-(2-bromo-5-fluorophenyl)-1-tert-butyl-4-iodo-1H-pyrazole

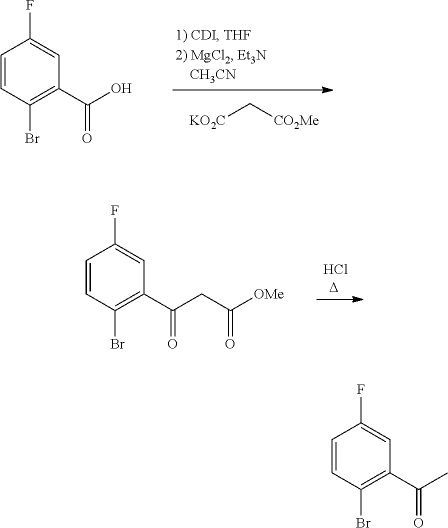

Readily available 2-bromo-5-fluorobenzoic acid was converted to methyl 3-(2-bromo-5-fluorophenyl)-3-oxopropanoate using condensation with 3-methoxy-3-oxopropanoate. Subsequent de-carboxylation gave 1-(2-bromo-5-fluorophenyl)ethanone, which was then converted to 5-(2-bromo-5-fluorophenyl)-1-tert-butyl-4-iodo-1H-pyrazole as described in Example 1.

### Example 3

### Synthesis of (R)-4-cyclopropyl-8-fluoro-5-(6-(trifluoromethyppyridin-3-ylsulfonyl)-4,5-dihydro-1H-pyrazolo[4,3-c]quinoline

3.1. (R,E)-N-(Cyclopropylmethylene)-2-methylpropane-2-sulfinamide

(R,E)-N-(cyclopropylmethylene)-2-methylpropane-2-sulfinamide was prepared through condensation of (R)-(+)-2-methyl-2-propanesulfinamide and cyclopropanecarboxaldehyde (1.15 eq) using CuSO₄ (2.25 eq) in CH₂C1₂ for 4-5 days at room temperature. The title compound was also prepared as described in Example 10.1.

3.2. (R)-N-((1R)-(5-(2-Bromo-5-fluorophenyl)-1-tert-butyl-1H-pyrazol-4-yl)(cyclopropyl)methyl)-2-methylpropane-2-sulfinamide

(R,E)-N -(cyclopropylmethylene)-2-methylpropane-2-sulfinamide was pretreated with approximately 10 mole % of i-PrMgCl at about −45° C. in order to remove any protic species which could quench the Grignard reagent. The mixture was added to (5-(2-bromo-5-fluorophenyl)-1-tert-butyl-1H-pyrazol-4-yl)magnesium chloride, which was prepared from 5-(2-bromo-5-fluorophenyl)-1-tert-butyl-4-iodo-1H-pyrazole (Example 1) by treatment with i-PrMgCl at about −10° C. to about 0° C. to affect a halogen-metal exchange. After slowly warming to room temperature, the reaction mixture was cooled to about −20° C. and quenched with a slight excess (1.2 eq relative to input of i-PrMgCl) of HOAc followed by warming to RT. After a conventional workup, N-((1R)-(5-(2-bromo-5-fluorophenyl)-1-tert-butyl-1H-pyrazol-4-yl)(cyclopropyl)methyl)-2-methylpropane-2-sulfinamide was obtained as a solution in MTBE/THF and was used without purification in the next process step.

The diastereoselectivity for the (R, R)-diastereomer was found to be consistent (about 97%) and the amount of recovered de-iodinated starting material, 5-(2-bromo-5-fluorophenyl)-1-tert-butyl-1H-pyrazole, which is an indicator of the coupling efficiency, was generally found to be less than 5% (typically between 2 and 4%).

3.3. (1R)-(5-(2-Bromo-5-fluorophenyl)-1-tert-butyl-1H-pyrazol-4-yl)(cyclopropyl)-methanamine

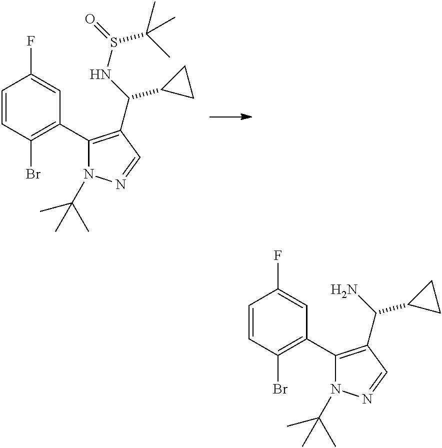

A solution of N-((1R)-(5-(2-bromo-5-fluorophenyl)-1-tert-butyl-1H-pyrazol-4-yl)(cyclopropyl)methyl)-2-methylpropane-2-sulfinamide in MTBE/THF was solvent exchanged into acetonitrile by distillation at reduced pressure. Subsequent treatment with 6 N HCl (2 eq) at about 0-10° C. resulted in complete deprotection in less than 1 hour. Dilution with water and extraction with toluene efficiently removed neutral impurities. The aqueous layer, after being made strongly basic with NaOH, was extracted with MTBE to afforded (1R)-(5-(2-bromo-5-fluorophenyl)-1-tert-butyl-1H-pyrazol-4-yl)(cyclopropyl)methanamine, as a mixture of atropisomers (about 40:60) by HPLC. The MTBE solution was dried by partial concentration at reduced pressure and was used without further purification for the next step.

3.4. N-((1R)-(5-(2-Bromo-4,5-difluorophenyl)-1-tert-butyl-1H-pyrazol-4-yl)(cyclopropyl)methyl)-6-(trifluoromethyl)pyridine-3-sulfonamide

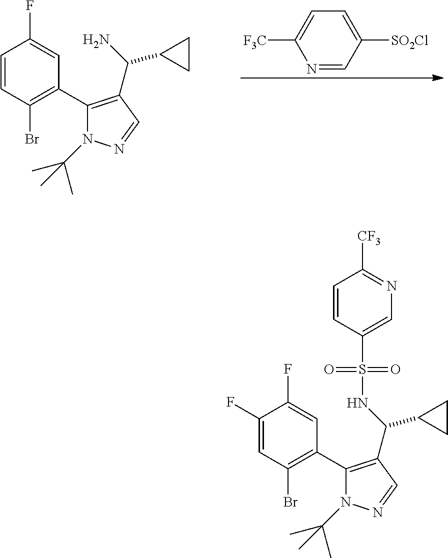

The above MTBE solution (1R)-(5-(2-bromo-5-fluorophenyl)-1-tert-butyl-1H-pyrazol-4-yl)(cyclopropyl)methanamine was treated with a slight excess of 6-(trifluoromethyl)pyridine-3-sulfonyl chloride (1.2 eq) and Et₃N (2 eq) at 0-10° C. and allowed to warm to RT until the reaction was complete by HPLC. The mixture was quenched with water to consume any residual sulfonyl chloride. The aqueous layer was discarded and the organic phase was washed with 0.5 M NaHSO₄, 1 N NaHCO₃, and brine. After concentration to a syrup and partial solidification, slurrying with a minimum amount of MTBE and filtration gave solid N-((1R)-(5-(2-bromo-4,5-difluorophenyl)-1-tert-butyl-1H-pyrazol-4-yl)(cyclopropyl)methyl)-6-(trifluoromethyl)pyridine-3-sulfonamide (45:55 mixture of atropisomers by HPLC) with the filtrate containing the same compound as a different mixture of atropisomers (35:65). The atropisomers do not interconvert, even at elevated temperature (vide infra), but since both cyclize to the same product, they were not isolated. The MTBE solution of the crude product was solvent swapped into toluene for use directly in the ring closure reaction described in Example 3.5. The chemical purity of the crude product was typically about 95% (HPLC) and further purification was not necessary.

3.5. (R)-1-tert-Butyl-4-cyclopropyl-8-fluoro-5-(6-(trifluoromethyl)pyridin-3-ylsulfonyl)-4,5-dihydro-1H-pyrazolo[4,3-c]quinoline

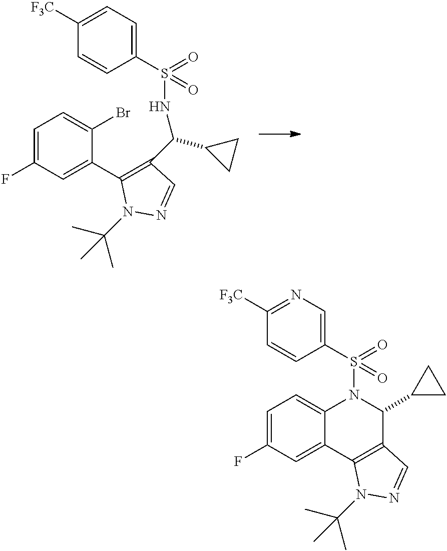

The above ring closure can be affected using stoichiometric amounts of CuI and CsOAc in DMSO at about 160° C. (see, e.g., US2008/0021056 and Angew. Chem. Int. Ed., 2003, 42, 5400-5449). However, the known procedure requires that a heated solution of the starting material be added to a preheated solution of CuI/CsOAc in a minimal amount of boiling DMSO. These reaction conditions were not amenable to large-scale preparations. When using other conditions, the product partly aromatized to a tricyclic-quinoline and significant de-bromination of the starting material was observed.

Iron-mediated (e.g., FeCl₃/DMEDA) and palladium-mediated couplings (e.g., Pd₂(dba)₃/Xantphos, Pd(OAc)₂/Xantphos, Pd(OAc)₂/BINAP, and Pd(OAc)₂/Cy₃P) were investigated to affect the cyclization of N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide. These efforts were unsuccessful leading to little or no conversion of the starting material.

In conjunction with CuI (20 mol %) and K₃PO₄ (2.2. eq) as the reagents various organic ligands were tested, including 1,10-phenanthroline, 2,2′-biquinoline, (E)-2-hydroxybenzaldehyde oxime, 8-hydroxyquinoline, picolinic acid, N,N-dimethylglycine, N-methylglycine, 2,2′-bipyridine, N,N-diethylsalicylamide (DESA), N,N′-dimethylcyclohexane-1,2-diamine and N,N-dimethylethane-1,2-diamine (DMEDA, J. Am. Chem. Soc., 2002, 124, 7421-7428). Certain 1,2-diamines, caused superior conversions (greater than 95%) without degradation of the reactants. In addition, with the improved catalytic system, common solvents, such as toluene could be used at moderate temperatures. Also, the amounts of certain impurities formed through aromatization and de-bromination were significantly reduced or eliminated. The crude product had sufficient purity to be carried forward to the next process step without chromatography.

Employing CuI (20 mol %), DMEDA (25 mol %), and 2.2 eq of K₃PO₄ in toluene at about 135° C. led to clean conversion to the desired product in less than 24 hours and essentially without the formation of degradation products. Additional ligands were explored using the above conditions as a reference. During the screen, DESA (Org. Lett., 2003, 5, 793-796) and N,N′-dimethyl-cyclohexane-1,2-diamine were found to be useful as well. Other tested ligands led to little or no conversion within the first 4 hours of reaction time.

Under otherwise identical conditions, DESA gave rise to very rapid reaction rates. Reactions were typically complete in less than 4 hours under otherwise identical conditions. However, unusual behavior regarding the rate of consumption of individual atropisomers of the starting material was observed. Reactions conducted using DMEDA as the ligand displayed little to no detectable atropisomer preference. In addition, reactions catalyzed by the CuI/DESA system were characterized by an initial “lag” period, with a gradual acceleration in reaction rate leading to an overall “parabolic” kinetic profile. Interestingly, reactions catalyzed by the CuI/DMEDA system displayed linear kinetics instead. Although N,N′-dimethyl-cyclohexane-1,2-diamine was about as effective as DMEDA, it was not explored further because the compound is significantly more expensive.

To further optimize the cyclization, additional exploration of the catalyst/ligand ratio in the case of the CuI/DMEDA system allowed the catalyst loading to be lowered to 2 mol % CuI in conjunction with 10 mol % DMEDA. The influence of the catalyst/ligand ratio in this reaction is quite pronounced, with reaction rates greatly enhanced by the inclusion of a sizeable excess of DMEDA ligand.

Optimized reaction conditions involved the use of 2 mol % Cut 10 mol % DMEDA, and 1.7 eq K₂CO₃ in toluene at about 135° C. The use of toluene as the solvent was chosen, even though the reaction required temperatures above its boiling point, because it forms an azeotrope with Me0H. This was particularly convenient because it allows for a direct solvent-swap from toluene into MeOH, from which the product crystallized in high purity. MeOH tended to give slightly better recoveries and therefore became the solvent of choice and was later incorporated into the final solvent swap/crystallization procedure (vide infra).

The optimized protocol for workup and isolation from the cyclization involves an initial filtration step to remove inorganic material, followed by treatment with a saturated aqueous solution of NH₄Cl for several hours to remove residual copper. Following a standard aqueous workup, the resulting toluene solution is concentrated by vacuum distillation (about140 torr, 65° C.) to a minimum volume. MeOH is then added and the distillation continued at atmospheric pressure until all of the toluene has been removed. Finally, the MeOH solution is reduced and allowed to cool, whereupon white crystals are deposited. The crystallized product contained very low levels of residual copper as determined by ICP-OES and the chemical purity was typically greater than 99.5%.

3.6. Recrystallization of (R)-1-tert-Butyl-4-cyclopropyl-8-fluoro-5-(6-(trifluoromethyl)pyridin-3-ylsulfonyl)-4,5-dihydro-1H-pyrazolo[4,3-e]quinoline

Although the above cyclization product was typically greater than 99% chemically pure (HPLC), the optical purity ranged from about 96% to about 97%. Thus, re-crystallizing was explored to increase the optical purity of the cyclized product. An initial solvent screen (including MeOH, MeOH/H₂O, EtOH/H₂O, IPA/H₂O, heptane, MeCN/H₂O, and acetone/H₂O) revealed MeOH/H₂O (10:1) to be the most promising solvent system in terms of both recovery and increase in optical purity.

3.7. (R)-4-cyclopropyl-8-fluoro-5-(6-(trifluoromethyl)pyridin-3-ylsulfonyl)-4,5-dihydro-1H-pyrazolo[4.3-c]quinoline

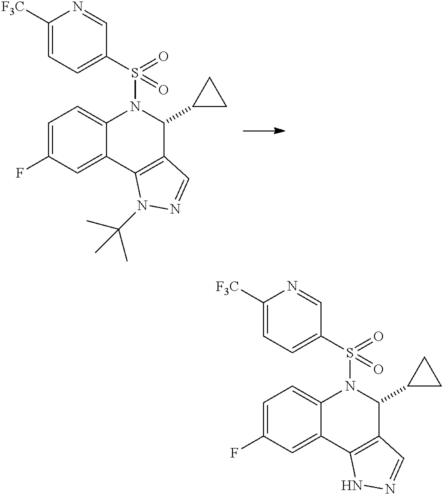

Deprotection of (R)-1-tert-butyl-4-cyclopropyl-8-fluoro-5-(6-(trifluoromethyl)pyridin-3-ylsulfonyl)-4,5-dihydro-1H-pyrazolo[4,3-c]quinoline was effected by heating in 5:1 formic acid:H₂O (6 volumes) at 60° C. for about 2 h. When the reaction mixture was cooled (<5° C.) and water was added dropwise, the product failed to precipitate as a solid. Several variations on the workup were examined in order to achieve a clean precipitation of the title compound from the reaction mixture. An optimized procedure includes (a) work-up in MTBE and solvent-swap into EtOH; (b) dropwise addition of ethanolic solution to cold (<5° C.) water, and resulted in an amorphous solid material in high chemical purity with 680 ppm EtOH and 20 ppm MTBE.

The formation of a single major impurity, (R)-2-tert-butyl-4-cyclopropyl-8-fluoro-5-(6-(trifluoromethyl)pyridin-3-ylsulfonyl)-4,5-dihydro-2H-pyrazolo[4,3-c]quinoline (tert-butyl regioisomer), was typically observed during the course of the deprotection reaction. Alternative reaction conditions were investigated that would diminish the rate of its formation. Four acidic solvent mixtures were examined as alternatives to 5:1 formic acid/H₂O. The use of methanolic HCl led to conversion to the desired product without formation of the previously observed impurity. However, when this reaction was stressed for a longer period of time (up to 24 h) several new impurities began to form. Acetic acid as the solvent led to no conversion after 2 h. Finally, two different concentrations of formic acid/H₂O were examined. A 1:1 system proved to be unsuitable, because starting material did not dissolve. The 2:1 formic acid/H₂O system, however, led to complete conversion in a slightly longer time than the 5:1 system. Even after stressing the reaction for 24 h, the impurity only formed to the extent of 0.8%, compared with 3-4% in the original 5:1 system.

Exemplary deprotection protocol: 40 g of compound 12 in a 1 L jacketed reactor were deprotected using 12 volumes of 2:1 formic acid/H₂O. The reaction mixture was heated to 60° C.; after heating at this temperature for 15 min, all of the solids had dissolved, and reaction sampling was then started at a rate of once every 30 min. The reaction was deemed complete after the fourth sampling (total reaction time at 60° C. of ˜2 h), and was then cooled to <25° C. The reaction mixture was taken up in MTBE (20 volumes). The organic phase was washed with H₂O (20 volumes×2), 1 M NaHCO₃ (20 volumes×2), and H₂O (20 volumes×2). Workups employing aqueous NaOH washes to remove the formic acid led to much slower phase splits. The MTBE solution was solvent-swapped into EtOH to a final concentration of ˜4 volumes. This solution was added dropwise (˜0.25 L/h) to a rapidly stirred (300 rpm) portion of cold (<5° C.) H₂O (40 volumes, i.e., a 10-fold excess relative to EtOH amount). The resulting fine, white precipitate was collected by filtration, and the cake was washed twice with cold (<5° C.) H₂O. The solids were transferred to a vacuum oven and dried at 50° C. until the weight remained constant to give the product in 97.5% yield and in greater than 99.5% chemical purity (ee: 97.9%).

Using the above described optimized procedures, (R)-4-cyclopropyl-8-fluoro-5-(6-(trifluoromethyl)pyridin-3-ylsulfonyl)-4,5-dihydro-1H-pyrazolo[4,3-c]quinoline is obtained from 5-(2-bromo-5-fluorophenyl)-1-tert-butyl-4-iodo-1H-pyrazole in at least 50% (e.g., about 54%) overall yield (mol/mol). The optimized process steps are summarized in Scheme 4, below.

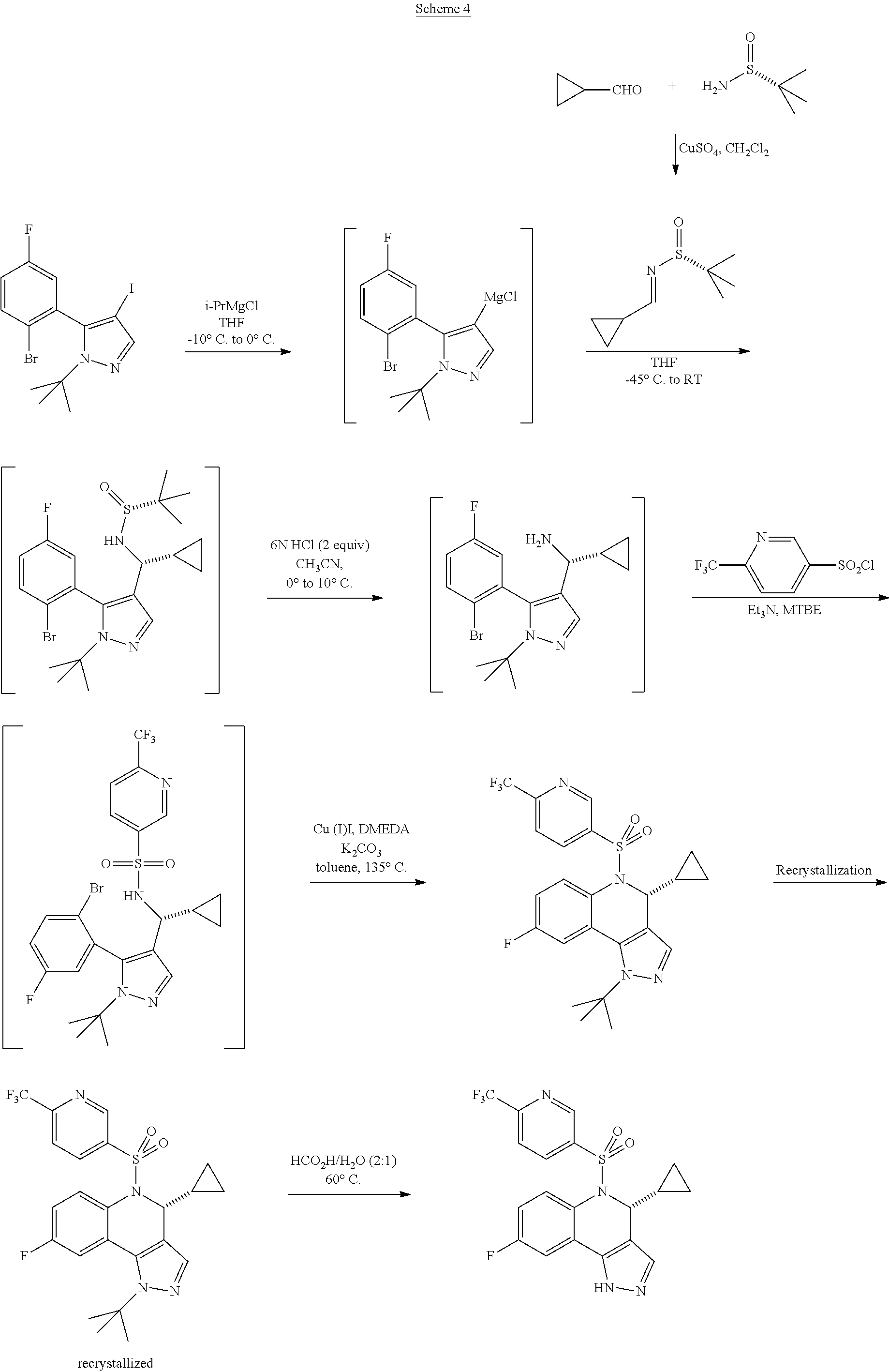

### Example 4

### Synthesis of (R)-4-cyclopropyl-8-fluoro-5-(6-(trifluoromethyppyridin-3-ylsulfonyl)-4,5-dihydro-1H-pyrazolo[4,3-c]quinoline

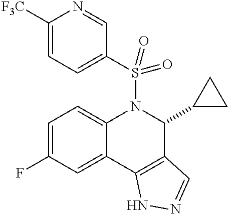

4.1. (R)-N-((1R)-(5-(2-bromo-5-fluorophenyl)-1-tert-butyl-1H-pyrazol-4-yl)(cyclopropyl)methyl)-2-methylpropane-2-sulfinamide

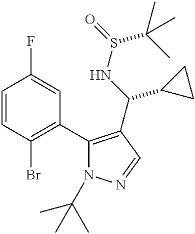

To a solution of (R,E)-N-(cyclopropylmethylene)-2-methylpropane-2-sulfinamide (0.45 kg, 2.6 moles) in THF (1.70 L) at −45° C. was slowly added isopropyl-magnesium chloride (0.14 kg, 0.28 moles) while maintaining the temperature below −40° C. The reaction mixture was stirred at −40 to −45° C. for at least 45 min (reaction mixture B).

To a solution of 5-(2-bromo-5-fluorophenyl)-1-tert-butyl-4-iodo-1H-pyrazole (1.0 kg, 2.36 moles) in THF (2.80 L) at −10 to −5° C. was slowly added isopropyl-magnesium chloride (1.15 kg, 2.36 moles) while maintaining the temperature below −2° C. The reaction mixture was stirred at −2° C. to 2° C. for about 90 min (reaction mixture A).

Reaction mixture A was slowly added to reaction mixture B while maintaining a temperature between −40° C. to −45° C. The reaction mixture was stirred at −40 to −45° C. for about 1 h and was then allowed to warm to 15° C. to 25° C. over 12 hours. It was stirred at this temperature for about 1 h before the reaction mixture was cooled to −25° C. to −20° C. and acetic acid (0.19 kg) was added while maintaining the reaction temperature below 0° C. Water (8.8 kg) was added and the aqueous phase was extracted twice with methyl tert-butyl (MTB) ether (8.66 L and 6.29 L). The combined organic phases were washed with water (6.29 kg), 2× with 1N sodium bicarbonate solution (6.3 L, 4.7) and saturated NaCl solution (4.7 L) to afford a solution of (R)-N-((1R)-(5-(2-bromo-5-fluorophenyl)-1-tert-butyl-1H-pyrazol-4-yl)(cyclopropyl)methyl)-2-methylpropane-2-sulfinamide, which was used without further purification in the next reaction step.

4.2. (1R)-(5-(2-bromo-5-fluorophenyl)-1-tert-butyl-1H-pyrazol-4-yl)(cyclopropyl)methanamine

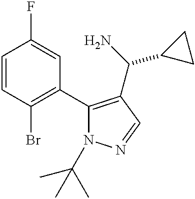

The above crude (R)-N-((1R)-(5-(2-bromo-5-fluorophenyl)-1-tert-butyl-1H-pyrazol-4-yl)(cyclopropyl)methyl)-2-methylpropane-2-sulfinamide was solvent swapped into acetonitrile using vacuum distillation (e.g., 400 mmHg, 40° C). to obtain an acetonitrile solution with a concentration of about 5.4-6.7 L solvent/kg crude product. To this solution at about 0° C. to about 10° C. was added 6 N HCl (0.79 L) and the reaction mixture was stirred at this temperature for about 0.5 h.

Water (20 kg) was added and the aqueous layer was washed with toluene (2×3.85 kg). It was then cooled to between about 0° C. and about 10° C. and the pH was adjusted to above 12 using 25% aqueous NaOH solution (about 1.13 kg). The product was extracted with MTB ether (3×2.55 kg). The combined organic phases were washed with saturated NaCl solution (6.3 L) and about 1.2 kg of the solvent was removed to afford an MTB ether solution of (1R)-(5-(2-bromo-5-fluorophenyl)-1-tert-butyl-1H-pyrazol-4-yl)(cyclopropyl)methanamine, which was used without further purification in the next reaction step. The yield was determined by drying an aliquot of this solution.

4.3. N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide

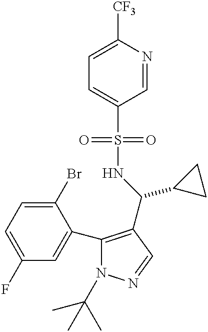

To the above solution of (1R)-(5-(2-bromo-5-fluorophenyl)-1-tert-butyl-1H-pyrazol-4-yl)(cyclopropyl)methanamine at 0° C. to 10° C. was added triethylamine (0.43 kg) and a solution of 6-(trifluoromethyl)pyridine-3-sulfonyl chloride (0.63 kg) in MTB ether (1.42 kg) while maintaining the reaction temperature between 0° C. and 10° C. The reaction mixture was stirred at this temperature for about 20 min, was then allowed to warm to room temperature (20° C. to 25° C.) and stirred at that temperature for another 0.5 h. Water (4.0 kg) was added and the mixture was stirred for about 2 h. The aqueous layer was discarded and the organic phase was washed with 0.5 M sodium bisulfate solution (2×4.0 L), 1N sodium bicarbonate solution (4.0 L) and saturated NaCl solution (2.5 L). The crude product was solvent swapped into toluene (e.g., 17.3 kg) using vacuum distillation (e.g., 140 mm Hg, 58° C.) to give a toluene solution of N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide, which was used without further purification in the next reaction step.

4.4. (R)-1-tert-butyl-4-cyclopropyl-8-fluoro-5-(6-(trifluoromethyl)pyridin-3-ylsulfonyl)-4,5-dihydro-1H-pyrazolo[4,3-c]quinoline

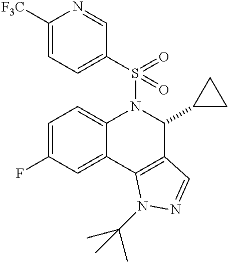

To the above toluene solution of N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide at 20° C. to 25° C. was added copper iodide (8 g), N,N′-dimethylethylenediamine (DMED) (19 g) and potassium carbonate (0.52 kg) and the reaction mixture was heated to between about 132° C. and 137° C. (e.g., 135° C.) at a pressure of less than about 2 bar. Under these conditions, the reaction mixture was stirred for 17 h and was then allowed to cool to room temperature. It was subsequently filtered through celite (0.25 kg). To the filtrate was added saturated ammonium chloride solution and the mixture was stirred for 4 h. The aqueous phase was discarded and the organic phase was washed with 1 N HCl (2×4.0 L) and water (3×4.0 kg). The crude product was solvent swapped into methanol using vacuum distillation to afford a solution containing about 4.0 L methanol/kg crude product. The methanol solution was cooled to between about 20° C. and 25° C. over 4 hours with gentle stirring to initialize crystallization and was held at this temperature for 2 hours. It was then further cooled to between about 0° C. and 5° C. and was held at that temperature for another 2 hours. The product was isolated by filtration, washed with cold MeOH/water (10:1 v/v, 2 L) and dried to a constant weight at 50° C. to give (R)-1-tert-butyl-4-cyclopropyl- 8-fluoro-5-(6-(trifluoromethyl)pyridin-3-ylsulfonyl)-4,5-dihydro-1H-pyrazolo[4,3-c]quinoline (0.86 kg, 1.74 mol, 74% overall yield from 5-(2-bromo-5-fluorophenyl)-1-tert-butyl-4-iodo-1H-pyrazole).

The above product (1 kg) was re-crystallized by first heating the material in methanol/water (10:1 v/v, 18.2 L, 1.8 L) to about 65° C. for at least 30 min with stifling until dissolved and by cooling the mixture to between about 50° C. and 55 ° C. over 1 h to initiate crystallization. The mixture was held at that temperature for about 1 h before it was further cooled to between about 20° C. and 25° C. over 3 h. The mixture was held at that temperature for another 3 h. The product was filtered off, washed with 2 L of cold methanol/water (10:1 v/v) and dried at 50° C. to afford purified (R)-1-tert-butyl-4-cyclopropyl-8-fluoro-5-(6-(trifluoromethyl)pyridin-3-ylsulfonyl)-4,5-dihydro-1H-pyrazolo[4,3-c]quinoline (0.76 kg, 76% re-crystallization yield).

4.5. (R)-4-cyclopropyl-8-fluoro-5-(6-(trifluoromethyl)pyridin-3-ylsulfonyl)-4,5-dihydro-1H-pyrazolo[4,3-c]quinoline

To a mixture of formic acid (8.0 L) and water (4.0 L) was added purified (R)-1-tert-butyl-4-cyclopropyl-8-fluoro-5-(6-(trifluoromethyl)pyridin-3-ylsulfonyl)-4,5-dihydro-1H-pyrazolo[4,3-c]quinoline (1.0 kg) and the reaction mixture was heated to about 60° C. with stifling until the starting material was dissolved and was then further heated for about 1 h. It was then rapidly cooled to about 20-25° C. (e.g., about 45 min). It was diluted with MTB ether (20 L) and washed with water (2×20 L) and 1N sodium bicarbonate solution (2×20 L, 2×1 h) (CO₂ formation). It was further washed with water (2×20 L) and the solvent was partially removed by vacuum distillation. The product was solvent swapped into ethanol to a final concentration of about 4 L ethanol/kg crude product and cooled to room temperature. The ethanolic solution was slowly (e.g., 0.25 L/h) added to 40 L of cooled (about 3° C.) deionized water. The mixture was stirred at about 5° C. for 3 h. The solid product was filtered off, washed with cold deionized water (5 L) and dried at 50° C. to give 0.84 kg of deprotected and purified (R)-4-cyclopropyl-8-fluoro-5-(6-(trifluoromethyl)pyridin-3-ylsulfonyl)-4,5-dihydro-1H-pyrazolo[4,3-c]quinoline. MS m/z 439 (M+H)⁺; 461(M+Na)⁺. ¹H NMR (CDCl₃) δ 10.13 (broad s, 1H), 8.51 (s, 1H), 7.85 (dd, J=12.0 and 6.4 Hz, 1H), 7.40 (m, 2H), 7.36 (d, J=3.0 Hz, 1H), 7.28 (s, 1H), 7.14 (dt, J=3.0 and 8.7 Hz, 1H), 4.99 (d, J=7.8 Hz, 1H), 1.01 (m, 1H), 0.54 (m, 1H), 0.39 (m, 2H), 0.09 (m, 1H).

### Example 5

### Synthesis of 5-(2-bromo-4,5-difluorophenyl)-1-tert-butyl-4-iodo-1H-pyrazole

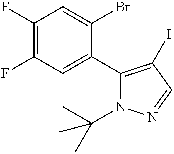

The title compound was prepared from 1-(2-bromo-4,5-difluorophenyl)ethanone according to the procedures outlined in Example 1. ¹H NMR (CDCl₃) δ 7.57 (s, 1H), 7.53 (m, 1H), 7.14 (m, 1H), 1.46 (s, 9H).

### Example 6

### Synthesis of (R)-1-tert-Butyl-4-cyclopropyl-7,8-difluoro-5-(4-(trifluoromethyl)phenylsulfonyl)-4,5-dihydro-1H-pyrazolo[4,3-c]quinoline

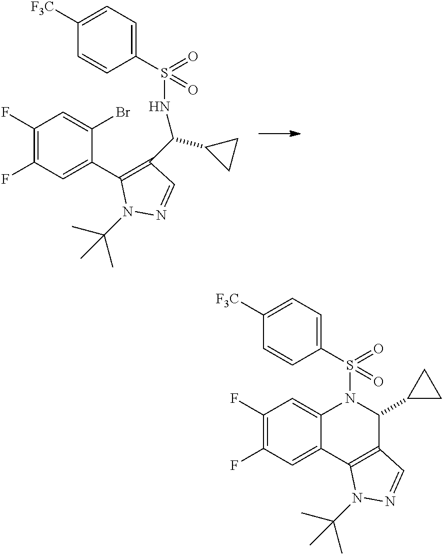

To a mixture of N-((1R)-(5-(2-bromo-4,5-difluorophenyl)-1-tert-butyl-1H-pyrazol-4-yl)(cyclopropyl)methyl)-4-(trifluoromethyl)benzenesulfonamide (412 mg, 0.686 mmol), copper (I) iodide (98% purity, 13 mg, 0.068 mmol, 10% loading) and potassium phosphate (295 mg, 1.35 mmol) in dry o-xylene (2 mL) under nitrogen was added N,N′dimethylethylenediamine (DMEDA, 31 mg, 37 uL, 0.35 mmol, about 50 mol %) and the mixture was heated at 130° C. for 15 h, after which time the starting material was consumed (LCMS). The mixture was cooled to ambient temperature and passed through a silica gel pad (eluted with 1:1 ethyl acetate/hexanes). The solvent was evaporated in vacuo to give (R)-1-tert-butyl-4-cyclopropyl-7,8-difluoro-5-(4-(trifluoromethyl)phenylsulfonyl)-4,5-dihydro-1H-pyrazolo[4,3-c]quinoline (280 mg, 78%) in 93% purity (LCMS) containing about 2% of aromatized quinoline and about 5% of de-brominated acyclic sulfonamide which was already present in the starting material. Note: The starting material was an approximately 55:45 mixture of rotational isomers. In this experiment each rotational isomer cyclized with equal efficiency.

### Example 7

### Synthesis of ((R)-4-cyclopropyl-7,8-difluoro-5-(4-(trifluoromethyl)phenylsulfonyl)-4,5-dihydro-1H-pyrazolo[4,3-c]quinoline

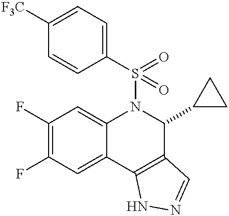

7.1. (R)-N-((1R)-(5-(2-bromo-4,5-difluorophenyl)-1-tert-butyl-1H-pyrazol-4-yl)(cyclopropyl)methyl)-2-methylpropane-2-sulfinamide

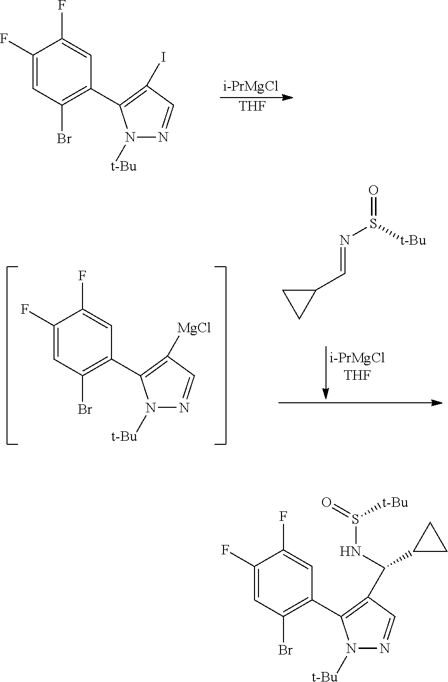

To a solution of (R,E)-N-(cyclopropylmethylene)-2-methylpropane-2-sulfinamide (0.43 kg, 2.48 moles) in dry THF (1.72 kg, 1.93 L) between about −45° C. and about −40 ° C. under nitrogen, was slowly added isopropyl-magnesium chloride (0.25 moles, 0.12 kg, 0.12 L) maintaining a temperature below −40° C. The reaction mixture was stirred at between about −45° C. and about −40° C. for at least 45 minutes (reaction mixture B).

To a solution of 5-(2-bromo-4,5-difluorophenyl)-1-tert-butyl-4-iodo-1H-pyrazole (1.0 kg, 2.27 moles) in THF (2.00 L) at −10 to −5° C. under nitrogen was slowly added isopropyl-magnesium chloride (1.11 kg, 2.27 moles) while maintaining the temperature below −2° C. The reaction mixture was stirred at −2° C. to 2° C. for about 1 hour (reaction mixture A).

Reaction mixture A was slowly added to reaction mixture B while maintaining a temperature between about −40° C. and −45° C. The reaction mixture was stirred at between about −40 and about −45° C. for about 1 h and was then allowed to warm to between about 15° C. and about 25° C. over 12 hours. It was stirred at this temperature for about 1 h and a sample was taken for analysis. The diastereoselectivity of the above “reverse Ellman” coupling was 95.6%. The reaction mixture was then cooled to between about −25° C. and about −20° C. and acetic acid (0.18 kg, 0.17 L) was added while maintaining a reaction temperature below 0° C. The reaction mixture was warmed to between about 15° C. and about 20° C. and methyl tert-butyl (MTB) ether (6.1 L; 4.5 kg) and tap water (8.5 L) were added. The mixture was stirred for about 15 min and was then filtered through a 0.45 μm filter cartridge. The aqueous phase was separated from the organic phase and was extracted with methyl tert-butyl (MTB) ether (6.1 L, 4.5 kg). The combined organic phases were washed with water (6 L), twice with 1N sodium bicarbonate solution (6.0 L and 4.5 L) and saturated NaCl solution (4.5 L). The combined organic phases were filtered through a 0.45 μm filter cartridge to afford a MTB ether solution of (R)-N-((1R)-(5-(2-bromo-4,5-difluorophenyl)-1-tert-butyl-1H-pyrazol-4-yl)(cyclopropyl)methyl)-2-methylpropane-2-sulfinamide, which was used without further purification in the next reaction step.

7.2. (1R)-(5-(2-bromo-4,5-difluorophenyl)-1-tert-butyl-1H-pyrazol-4-yl)(cyclopropyl)methanamine

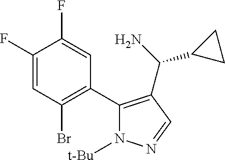

The above crude (R)-N-((1R)-(5-(2-bromo-4,5-difluorophenyl)-1-tert-butyl-1H-pyrazol-4-yl)(cyclopropyl)methyl)-2-methylpropane-2-sulfinamide was solvent swapped into acetonitrile using vacuum distillation (e.g., 400 mm Hg, 40° C). to obtain an acetonitrile solution with a concentration of about 5.2-6.4 L solvent/kg crude product. To the solution at between about 0° C. and about 10° C. was added 6 N HCl (0.76 L) while maintaining the temperature between about 0° C. and about 10° C. The reaction mixture was stirred at this temperature for about 0.5 h, at which time the deprotection was complete (HPLC).

Water (18.1 kg) was added and the aqueous layer was washed with toluene (2×3.9 kg). It was then cooled to between about 0° C. and about 10° C. and the pH was adjusted to above 12 using 25% aqueous NaOH solution (about 1.13 kg). The product was extracted with MTB ether (3×3.8 L). The combined organic phases were washed with saturated NaCl solution (2.0 L) and about 1.2 kg of the solvent was removed to afford an MTB ether solution of (1R)-(5-(2-bromo-4,5-difluorophenyl)-1-tert-butyl-1H-pyrazol-4-yl)(cyclopropyl)methanamine, which was used without further purification in the next reaction step. The yield was determined by drying an aliquot of this solution (700.43 g, 1.823 mol, 80.3% yield).

7.3. N-((1R)-(5-(2-bromo-4,5-difluorophenyl)-1-tert-butyl-1H-pyrazol-4-yl)(cyclopropyl)methyl)-4-(trifluoromethyl)benzenesulfonamide

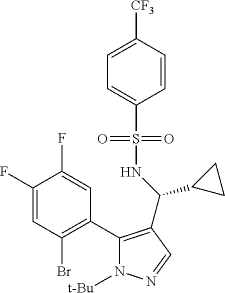

To the above MTB ether solution of (1R)-(5-(2-bromo-4,5-difluorophenyl)-1-tert-butyl-1H-pyrazol-4-yl)(cyclopropyl)methanamine at 0° C. to 10° C. was added triethylamine (0.38 kg) and a solution of 4-(trifluoromethyl)benzene-1-sulfonyl chloride (0.55 kg) in MTB ether (1.36 kg) while maintaining a reaction temperature between about 0° C. and about 10° C. The reaction mixture was stirred at this temperature for about 20 min and was then allowed to warm to room temperature (between about 20° C. and about 25° C.). Water (3.8 kg) was added and the mixture was stirred for about 2 h in order to hydrolyze residual sulfonyl chloride. The aqueous layer was discarded and the organic phase was washed with 0.5 M sodium bisulfate solution (2×3.8 L), 1N sodium bicarbonate solution (2×3.8 L) and saturated NaCl solution (2.3 L). The crude product was solvent swapped into toluene (e.g., 16.6 kg) using vacuum distillation (e.g., 140 mm Hg, 90° C.) to give a toluene solution of N-((1R)-(5-(2-bromo-4,5-difluorophenyl)-1-tert-butyl-1H-pyrazol-4-yl)(cyclopropyl)methyl)-4-(trifluoromethyl)benzene sulfonamide, which was used without further purification in the next reaction step.

7.4. (R)-1-tert-butyl-4-cyclopropyl-7,8-difluoro-5-(4-(trifluoromethyl)phenylsulfonyl)-4,5-dihydro-1H-pyrazolo[4,3-e]quinoline

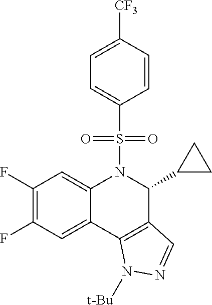

To the above toluene solution of N-((1R)-(5-(2-bromo-4,5-difluorophenyl)-1-tert-butyl-1H-pyrazol-4-yl)(cyclopropyl)methyl)-4-(trifluoromethyl)benzenesulfonamide at 20° C. to 25° C. was added copper iodide (7 g) and N,N′-dimethylethylenediamine (DMED) (16 g) and potassium carbonate (0.44 kg) and the reaction mixture was heated to between about 132° C. and 137° C. (e.g., 135° C.) for 14 h at a pressure of less than about 2 bar. The batch was cooled to less than 60° C. for sampling. The reaction mixture still contained starting material and another 7 g of copper iodide and 16 g of DMED were added in two batches. The reactor was cooled to between about 20° C. and about 25° C. for each addition, and after each addition, heating to between about 132° C. and 137° C. was continued (about 10 h each). The reaction mixture was then allowed to cool to room temperature and was subsequently filtered through celite (0.23 kg). The filtrate was further filtered through a 1.0 μm filter bag and a 0.45 μm filter cartridge.

To the filtrate was added saturated ammonium chloride solution (3.8 L) and the mixture was stirred for 4 h. The aqueous phase was discarded and the organic phase was washed with 1 N HCl (2×3.8 L) and water (3×3.8 L) to give a pale yellow solution. The crude product was solvent swapped into methanol (about 15.2 L) using vacuum distillation (e.g., 150 mm Hg, 64° C.) to afford a solution containing about 12 L methanol/kg crude product and less than 1% toluene by GC. To the methanol solution was added water (0.9 kg) and the mixture was cooled to between about 20° C. and about 25° C. over 4 hours while stifling slowly to affect crystallization. Crystallization occurred at about 48° C. Additional water (0.9 kg) was added and the mixture was stirred slowly for about 1 h. It was then cooled to between about 0° C. and about 5° C. over 2 hours and was stirred for another hour at this temperature. The solid product was obtained by filtration through an oyster filter equipped with a 3-5 μm filter cloth. The filter cake was washed with cold MeOH/water (5:1 v/v, 2 L) and dried to a constant weight at 50° C. (about 29 h) to give (R)-1-tert-butyl-4-cyclopropyl-7,8-difluoro-5-(4-(trifluoromethyl)-phenylsulfonyl)-4,5-dihydro-1H-pyrazolo[4,3-c]quinoline (0.764 kg, 1.49 mol, 66% overall yield starting from 5-(2-bromo-4,5-difluorophenyl)-1-tert-butyl-4-iodo-1H-pyrazole).

7.5. (R)-4-Cyclopropyl-7,8-difluoro-5-(4-(trifluoromethyl)phenylsulfonyl)-4,5-dihydro-1H-pyrazolo[4,3-c]quinoline

To a mixture of formic acid (12.0 L) and water (4.0 L) was added the above (R)-1-tert-butyl-4-cyclopropyl-7,8-difluoro-5-(4-(trifluoromethyl)phenylsulfonyl)-4,5-dihydro-1H-pyrazolo[4,3-c]quinoline (1.0 kg) and the reaction mixture was heated to about 60° C. with stifling until the starting material was dissolved and was then further heated for about 1.5 hours. It was then rapidly cooled to about 20-25° C. (e.g., about 45 min). Crystallization was observed at about 58° C. It was further cooled to between about 0° C. and about 10° C. To the mixture was then slowly added 25 L of water while stirring vigorously and while maintaining a temperature of between about 0° C. and about 10° C. The mixture was held at between about 3° C. and about 7° C. for another hour before the solids were filtered off. The filter cake was washed with 2×1.5 kg of water and dried to a constant weight at less than 60° C. to give (R)-4-cyclopropyl-7,8-difluoro-5-(4-(trifluoromethyl)phenylsulfonyl)-4,5-dihydro-1H-pyrazolo[4,3-c]quinoline as a white powder (883.5 g, 1.94 mol, 99% yield, 98% AUC purity by HPLC and 93% AUC optical purity by HPLC).

7.6. Purified (R)-4-cyclopropyl-7,8-difluoro-5-(4-(trifluoromethyl)phenylsulfonyl)-4,5-dihydro-1H-pyrazolo[4,3-e]quinoline

A suspension of the above (R)-4-cyclopropyl-7,8-difluoro-5-(4-(trifluoromethyl)phenylsulfonyl)-4,5-dihydro-1H-pyrazolo[4,3-c]quinoline (1 kg) in dichloromethane (6.6 kg) was heated to reflux for about 1 hour and was then cooled to between about 20° C. and about 25° C. Potassium carbonate (325 mesh, 0.3 kg) was added and the mixture was held at this temperature for about 1 hour. The solids were filtered off using a 0.45 μm filter bag and a 0.45 μm filter cartridge. The filtrate was solvent swapped into absolute ethanol (less than 50 ppm dichloromethane by GC) by distillation to a final volume of about 3.4 L. The ethanolic solution was cooled to about 40° C. and deionized water (2.1 kg) was added slowly (e.g., about 1 h) while maintaining the temperature at about 40° C. An additional 3.0 kg of deionized water was slowly added and the mixture was then cooled to about 20° C. for 1 hour. The solid product was filtered off, washed with cold deionized water (3.4 kg) and dried at not more than 60° C. to constant weight to give purified (R)-4-cyclopropyl-7,8-difluoro-5-(4-(trifluoromethyl)phenylsulfonyl)-4,5-dihydro-1H-pyrazolo[4,3-c]quinoline as a white powder (715.6 g, 1.57 mol, 81% yield, 99.8% AUC purity by HPLC and 99.8% AUC optical purity by HPLC). MS m/z 456.0(M+H)⁺, 478(M+Na)⁺. ¹H NMR (CDCl₃) δ 7.76 (dd, J=11.1, 7.2 Hz, 1H), 7.47 (m, J=10.2, 8.7 Hz, 1H), 7.39 (d, J=8.7 Hz, 2H), 7.32 (d, J=8.7 Hz, 2H), 7.20 (s, 1H), 4.95 (d, J=7.8 Hz, 1H), 1.03 (m, 1H), 0.54 (m, 1H), 0.41 (m, 2H), 0.08 (m, 1H).

In this example, the overall yield for the preparation of purified (R)-4-cyclopropyl-7,8-difluoro-5-(4-(trifluoromethyl)phenylsulfonyl)-4,5-dihydro-1H-pyrazolo[4,3-c]quinoline from 5-(2-bromo-4,5-difluorophenyl)-1-tert-butyl-4-iodo-1H-pyrazole was about 53% mol/mol.

### Example 8

### Synthesis of 1-tert-butyl-4-iodo-5-(2,4,5-trifluorophenyl)-1H-pyrazole

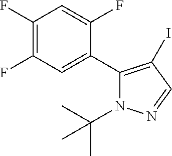

8.1. 1-(2,4,5-trifluorophenyl)-3-(dimethylamino)prop-2-en-1-one

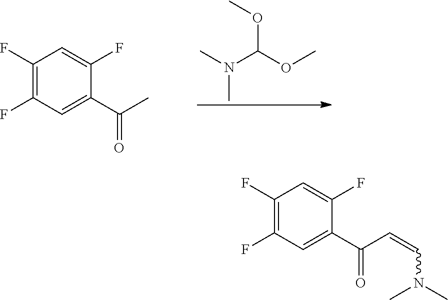

A mixture of 2′,4′,5′-trifluoroacetophenone (386 g, 2.21 mol) and DMF-DMA (1,1-dimethoxy-N,N-dimethylmethanamine, 791 g, 900 mL, 6.6 mol) was stirred and heated to a gentle reflux for 3 h. The solvent was removed under vacuum to give an orange crystalline solid (517 g). The product was used immediately in the next step. ¹H-NMR (CDCl₃); δ: 1.49 (s, 9H), 6.18 (d, 1H), 6.92-7.05 (m, 1H), 7.12-7.20 (m, 1H), 7.56 (narrow d, 1H). ¹³C-NMR (CDCl₃); δ: 30.54, 61.36, 105.37, 105.65, 105.75, 106.02, 110.08, 119.99, 120.75, 133.22, 137.03, 147.75. LC-MS m/z 255.0 (M+H)⁺.

8.2. 5-(2,4,5-trifluorophenyl)-1-tert-butyl-1H-pyrazole

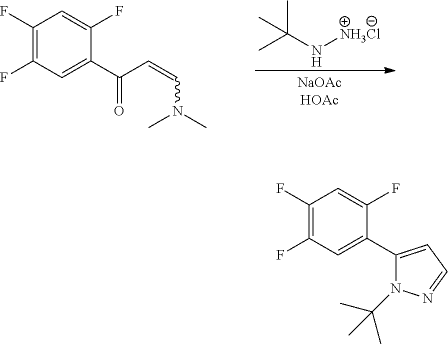

To a solution of the above crude 1-(2,4,5-trifluorophenyl)-3-(dimethylamino)prop-2-en-1-one (2.2 mol) in a mixture of dichloromethane (300 mL) and glacial acetic acid (1.0 L) under nitrogen was added additional acetic acid (1.0 L) and anhydrous sodium acetate (501 g, 6.1 mol). After the temperature dropped to about 36° C., tert-butyl hydrazine hydrochloride (359 g, 2.87 mol) was added as a solid in portions. The orange slurry was stirred at ambient temperature for about 60 h. The slurry was concentrated to about half of the initial volume and the residual solution was diluted with hexane:ethyl acetate (4:1, 4 L) and washed with water (4 L). The organic phase was separated and the aqueous layer was extracted with hexane:ethyl acetate (4:1, 2×2 L). The combined organic extracts were washed with water (2×2 L), saturated aqueous sodium bicarbonate (2×2 L), brine (1×1 L) and dried with anhydrous sodium sulfate. The solution was filtered and evaporated to dryness to give 560 g of a dark orange liquid.

¹H-NMR (CDCl₃); δ: 1.48 (s, 9H), 7.04-7.14 (m, 2H), 7.59 (s, 1H). ¹³C-NMR (CDCl₃); δ: 30.29, 62.49, 65.15, 105.86, 106.13, 106.23, 106.50, 120.05, 120.75, 135.93, 142.03. LC-MS m/z 380.9 (M+H)⁺.

8.3. 1-tert-Butyl-4-iodo-5-(2,4,5-trifluorophenyl)-1H-pyrazole

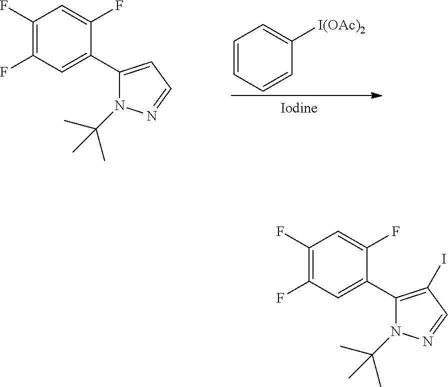

To a mixture of iodine (293 g, 1.15 mol, 0.53 eq) and DCM (1 L) under nitrogen was added iodosobenzene diacetate (380 g, 1.12 mol). The mixture was stirred for 25 min before a solution of 5-(2,4,5-trifluorophenyl)-1-tert-butyl-]H-pyrazole (558 g, 2.19 mol) in dichloromethane (1.2 L) was added. The dark mixture was stirred for 30 min. The solution was washed with aqueous sodium thiosulfate (3×500 mL), saturated aqueous sodium bicarbonate (2×500 mL) and brine (500 mL), and the organic layer was dried with anhydrous sodium sulfate. The solvent was removed to give crude 1-tert-butyl-4-iodo-5-(2,4,5-trifluorophenyl)-1H-pyrazole (904 g), which was crystallized from hexane. NMR (CDCl₃); δ: 1.48 (s, 9H), 7.04-7.14 (m, 2H), 7.59 (s, 1H). ¹³C-NMR (CDCl₃); δ: 30.29, 62.49, 65.15, 105.86, 106.13, 106.23, 106.50, 120.05, 120.75, 135.93, 142.03. LC-MS m/z 380.9 (M+H)⁺.

### Example 9

### Synthesis of (R)-4-cyclopropyl-7,8-difluoro-5-(4-(trifluoromethyl)phenylsulfonyl)-4,5-dihydro-1H-pyrazolo[4,3-c]quinoline

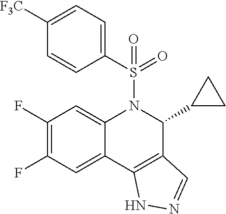

9.1. 1-tert-Butyl-5-(2,4,5-trifluorophenyl)-1H-pyrazole-4-carbaldehyde

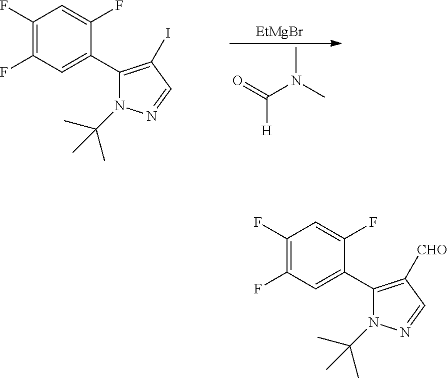

To a solution of 1-tert-butyl-4-iodo-5-(2,4,5-trifluorophenyl)-1H-pyrazole (385 g, 1.01 mol) in anhydrous THF (750 mL) at about 2° C. was added a solution of ethyl magnesium bromide (3M in THF, 398 mL, 1.19 mmol) over 45 min. The reaction mixture was stirred for an additional 60 minutes. An additional amount of ethyl magnesium bromide (60 mL, 0.18 mol) was added and stifling was continued for an additional 40 min. Anhydrous DMF (235 mL, 3.04 mol) was added over 25 min maintaining the temperature between about 4° and about −5° C. The reaction was stirred at −5° C. for 70 min, then aqueous ammonium chloride (half saturated, 300 mL) was added and stifling was continued for an additional 1 h. The inorganic salts separated, forming a slurry while the organic phase separated as a clear yellow liquid. The organic solution was decanted and the residue was washed with ethyl acetate (5×300 mL) and decanted. The combined organic solutions were washed with brine (650 mL) and dried with anhydrous sodium sulfate. The solution was filtered and evaporated to dryness to give a pale yellow solid (275 g). ¹H-NMR (CDCl₃); δ: 1.48 (s, 9H), 7.04-7.14 (m, 2H), 7.59 (s, 1H). ¹³C-NMR (CDCl₃); δ: 30.29, 62.49, 65.15, 105.86, 106.13, 106.23, 106.50, 120.05, 120.75, 135.93, 142.03. LC-MS m/z 310.0 (M+H)⁺.

9.2. (2S)-N-((1-tert-Butyl-5-(2,4,5-trifluorophenyl)-1H-pyrazol-4-yl)methylene)-2-methylpropane-2-sulfinamide

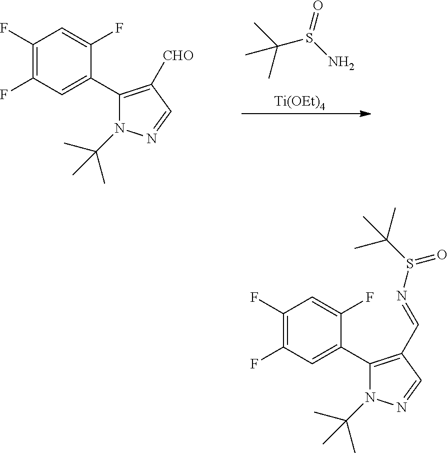

To a solution of 1-tert-butyl-5-(2,4,5-trifluorophenyl)-1H-pyrazole-4-carbaldehyde (275 g, 0.975 mol) in anhydrous THF (1.5 L) under nitrogen at ambient temperature was added titanium tetraethoxide (neat, 300 mL, 1.42 mol) followed within 10 min, by solid S-tert-butyl-sulfinamide (139 g, 1.14 mol). The yellow solution was stirred at ambient temperature for 18 h. The reaction mixture was poured slowly into stirred brine (2 L). The slurry was stirred for 45 min and was then filtered through a celite pad (3×8 in) using ethyl acetate to rinse the filter pad. The layers were separated and the aqueous layer was extracted with ethyl acetate. The combined organic layers were washed with brine, dried with anhydrous sodium sulfate, filtered and evaporated to give a yellow syrup (410 g), which was dissolved in hexane (700 mL) containing a small amount of ethyl acetate (approx. 100 mL, used to speed dissolution). The yellow solution was filtered through a pad of silica gel, which was washed with hexane and hexane:ethyl acetate (5:1). The filtrates were evaporated to give a yellow oil (362 g). ¹H-NMR (CDCl₃) δ: 1.10 (two poorly resolved s, 9H), 1.46 (s, 9H), 6.97-7.22 (m, 2H), 7.97 (d, 1H), 8.13 (d, 1H). ¹³C-NMR (CDCl₃); δ: 22.27, 30.49, 57.13, 57.22, 62.81, 106.71, 119.88, 120.17, 120.43, 138.28, 138.51, 153.90, 153.98, 183.89.

9.3. N-((1R)-(1-tert-butyl-5-(2,4,5-trifluorophenyl)-1H-pyrazol-4-yl)(cyclopropyl)methyl)-2-methylpropane-2-sulfinamide

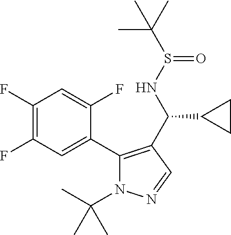

To a solution of (2S)-N-((1-tert-butyl-5-(2,4,5-trifluorophenyl)-1H-pyrazol-4-yl)methylene)-2-methylpropane-2-sulfinamide (111.0 g, 289 mmol) in DCM (1 L) under nitrogen at −74° C. was dropwise added cyclopropyl magnesium bromide solution (ca. 0.75 M in THF, 1550 mL, 1.15 mol) was added, maintaining a temperature of −74° to −73° over about 8.5 h. The mixture was stirred at −74° C. for 0.5 h. Aqueous ammonium chloride solution (ca. 500 mL) was added while the temperature rose to about −30°. The slurry was slowly warmed to ambient temperature. The yellow organic solution was decanted from a viscous residue. The residue was triturated with ethyl acetate (3×300 mL). The combined organic solutions were washed with brine (1×100 mL) and dried with anhydrous sodium sulfate. The solution was filtered and evaporated to give 433 g of a viscous, yellow oil. ¹H-NMR (CDCl₃); δ: −0.04-0.09 (m), 0.18-0.29 (m), 0.31-0.66 (m), 1.17 (s), 1.23 (s), 1.47 (s), 2.99-3.08 (m), 3.35-3.36 (m), 3.46-3.47 (m), 3.51-3.90 (m), 6.17-6.18 (m), 6.96-7.18 (m), 7.20-7.31 (m), 7.51-7.70 (m). LC-MS m/z 428 (M+H)⁺.

The title compound was also prepared from 1-tert-butyl-4-iodo-5-(2,4,5-trifluorophenyl)-1H-pyrazole using the procedures outlined in Example 4.

9.4. (1R)-(1-tert-butyl-5-(2,4,5-trifluorophenyl)-1H-pyrazol-4-yl)(cyclopropyl)-methanamine

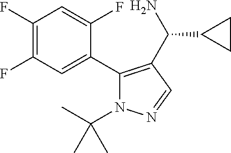

To a solution of N-((1R)-(1-tert-butyl-5-(2,4,5-trifluorophenyl)-1H-pyrazol-4-yl)(cyclopropyl)methyl)-2-methylpropane-2-sulfinamide (431 g) in methanol (1.4 L, anhydrous) at 3° C. was dropwise added HCl solution (4M in dioxane, 340 mL) over 35 min. The solution was stirred for an additional 25 min. The reaction mixture was concentrated under reduced pressure to remove most of the methanol and the residue was diluted with water (1 L). The dark aqueous solution was extracted with a hexane:ether (1:1, 3×500 mL). The aqueous phase was carefully adjusted to pH 12 with NaOH (ca. 5%) and extracted with ether (3×900 mL). The combined organic extracts were washed with brine (1×300 mL), dried with anhydrous sodium sulfate, filtered and evaporated to give an amber oil (254 g). ¹H-NMR (CDCl₃); δ: −0.04 to −0.006 (m, 1H), 0.06-0.16 (m, 1H), 0.32-0.54 (m, 2H), 0.93-1.11 (m, 1H), 1.45 (s, 9H), 2.67-2.71 (dd, 1H), 6.97-7.18 (m, 2H), 7.58-7.63 (d, 1H). ¹³C-NMR (CDCl₃); δ: 2.97, 3.12, 3.47, 3.64, 19.02, 19.38, 28.23, 30.49, 30.55, 51.59, 51.77, 56.03, 61.24, 105.52, 105.57, 105.80, 105.89, 105.95, 106.18, 120.42, 120.62, 120.78, 121.05, 127.66, 127.85, 130.19, 134.92, 134.99, 152.47. LC-MS m/z 324 (M+H)⁺.

9.5. N-((1R)-(1-tert-Butyl-5-(2,4,5-trifluorophenyl)-1H-pyrazol-4-yl)(cyclopropyl)methyl)-4-(trifluoromethyl)benzenesulfonamide

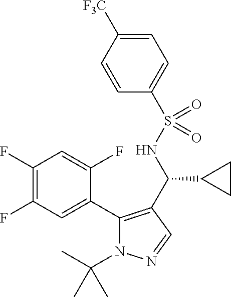

To a solution of the above (1R)-(1-tert-butyl-5-(2,4,5-trifluorophenyl)-1H-pyrazol-4-yl)(cyclopropyl)methanamine (213 g; 659 mmol) in DCM (1.4 L) was added triethylamine (400 mL, 2.97 M) and the mixture was cooled under nitrogen to approximately 3° C. To the mixture was added 4-(trifluoromethyl)phenyl sulfonyl chloride (212 g, 867 mmol, 1.3 eq) and the reaction mixture was allowed to warm to ambient temperature overnight (20 h). The reaction mixture was washed with water (3×1 L), saturated aqueous sodium bicarbonate (2×500 mL), water (1×1 L), 0.2N citric acid (2×1 L) and water (1×1 L) and was dried with anhydrous sodium sulfate, filtered and evaporated to give 347 g of a viscous oil. ¹H-NMR (CDCl₃); δ: −0.03-0.18 (m, 2H), 0.30-0.49 (m, 2H), 0.89-1.10 (m, 1H), 1.46 (s, 9H), 3.33-3.52 (m, 1H), 5.11-5.43 (m, 1H), 6.8-6.97 (m, 2H), 7.57-7.83 (m, 4H). LC-MS m/z 531 (M+H)⁺.

9.6. (R)-1-tert-butyl-4-cyclopropyl-7,8-difluoro-5-(4-(trifluoromethyl)phenylsulfonyl)-4,5-dihydro-1H-pyrazolo[4,3-e]quinoline

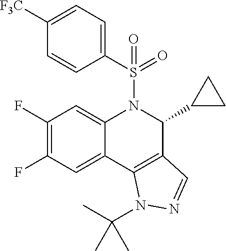

A solution of N-((1R)-(1-tert-butyl-5-(2,4,5-trifluorophenyl)-1H-pyrazol-4-yl)(cyclopropyl)methyl)-4-(trifluoromethyl)benzenesulfonamide (345 g, 0.58 mol) in anhydrous dimethylacetamide (2 L) was treated with cesium carbonate (498 g, 1.53 mol) and heated under nitrogen to between about 120 and 125° C. for 8 h. The mixture was cooled to ambient temperature and concentrated at 60°/4 mm Hg. To the residue were added ethyl acetate (4 L) and water (4 L). The organic layer was separated and the aqueous layer was extracted with ethyl acetate (2×500 mL). The combined organic extracts were washed with brine, dried with anhydrous sodium sulfate, filtered and evaporated to give 296 g of a brown, glassy residue. LCMS m/z 511 (M+H)⁺.

9.7. (R)-4-cyclopropyl-7,8-difluoro-5-(4-(trifluoromethyl)phenylsulfonyl)-4,5-dihydro-1H-pyrazolo[4,3-e]quinoline

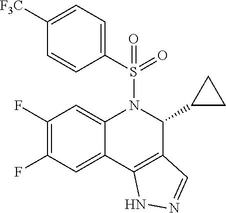

A solution of (R)-1-tert-butyl-4-cyclopropyl-7,8-difluoro-5-(4-(trifluoromethyl) phenylsulfonyl)-4,5-dihydro-1H-pyrazolo[4,3-c]quinoline (291 g, 0.56 mol) in formic acid (1250 mL) was stirred under nitrogen at 60° C. for 60 min. The mixture was cooled to ambient temperature and concentrated under reduced pressure to dryness. The dark brown residue was diluted with DCM (300 mL) and filtered through a plug of silica gel (4×8 in; settled in hexane) using hexane:DCM 1:2 (1.5 L), 1:1 (1.5 L) mixture, followed by DCM (2 L). The collected fractions were combined and concentrated to dryness to give a pale yellow oil (180 g), which was treated with hot DCM (about 250 mL) for 15 min and then let stand for 30 min. Precipitated colorless solids were filtered off, rinsed with cold DCM (100 mL) and air dried (24.9 g).

To a stirred solution of the above solids in DCM (200 mL) at ambient temperature was slowly added hexane (450 mL over 1 h). A fine precipitate appeared after about 1 to 2 h and the mixture was stirred for another 10 h. The solids were filtered off, washed with hexane (2×100 mL) and air dried to give (R)-4-cyclopropyl-7,8-difluoro-5-(4-(trifluoromethyl)phenylsulfonyl)-4,5-dihydro-1H-pyrazolo[4,3-c]quinoline as a white powder (64 g; mp. 137-138° C.). The filtrate was concentrated and treated as above to obtain a second crop of an off-white powder (13.9 g). MS m/z 456.0 (M+H)⁺, 478 (M+Na)⁺. ¹H NMR (CDCl₃) δ 7.76 (dd, J=11.1, 7.2 Hz, 1H), 7.47 (m, J=10.2, 8.7 Hz, 1H), 7.39 (d, J=8.7 Hz, 2H), 7.32 (d, J=8.7 Hz, 2H), 7.20 (s, 1H), 4.95 (d, J=7.8 Hz, 1H), 1.03 (m, 1H), 0.54 (m, 1H), 0.41 (m, 2H), 0.08 (m, 1H).

### Example 10

### Alternate synthesis of (R)-N-((1R)-(1-tert-Butyl-5-(2,4,5-trifluorophenyl)-1H-pyrazol-4-yl)(cyclopropyl) methyl)-2-methylpropane-2-sulfinamide

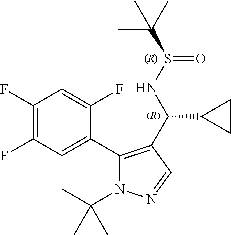

10.1. (E)-N-(Cyclopropylmethylene)-2-methylpropane-2-(R)-sulfinamide

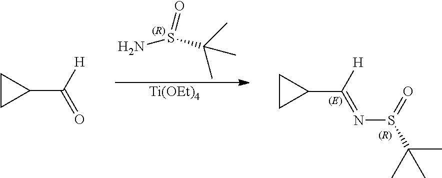

A solution of cyclopropane-carboxyaldehyde (6.6 g, 0.094 mol) in anhydrous THF (100 mL) was treated with titanium tetraethoxide (30 mL, 0.144 mol) and (R)-2-methylpropane-2-sulfinamide (13.85 g, 0.114 mol). The pale-yellow solution was stirred at ambient temperature for 20 h. The mixture was poured into stirred brine (200 mL) and stirring was continued for 1 h. The suspension was filtered through a Celite pad (2×2 in) and the cake was washed with EtOAc (200 mL) and THF (200 mL). After separation of the aqueous layer, the filtrate was stripped under reduced pressure, diluted with EtOAc (250 mL), washed with brine, dried with anhydrous sodium sulfate, filtered and evaporated to give 18.6 g of colorless oil. The crude product was diluted with hexane (25 mL) and filtered through a pad of silica gel (1×1 in) in hexane. The pad was washed with 500 mL of EtOAc:hex 1:2. The filtrate was evaporated to give (R,E)-N-(cyclopropylmethylene)-2-methylpropane-2-sulfinamide (14.9 g, 91%) as a colorless oil.

¹H-NMR (CDCl₃; δ): 0.89 (m, 2H), 1.02 (m, 2H), 1.12 (s, 9H), 1.92 (m, 1H), 7.38 (d, 1H). ¹³C-NMR (CDCl₃; δ): 8.40, 8.51, 17.54, 22.14, 22.19, 56.55, 171.63. LC-MS: m/z=174, 100%.

10.2. (R)-N-((1R)-(1-tert-butyl-5-(2,4,5-trifluorophenyl)-1H-pyrazol-4-yl)(cyclopropyl)methyl)-2-methylpropane-2-sulfinamide

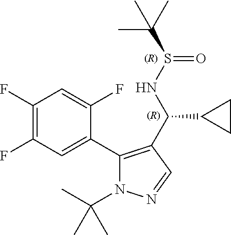

A solution of 1-tert-butyl-4-iodo-5-(2,4,5-trifluorophenyl)-1H-pyrazole (5.04 g, 13.3 mmol) in anhydrous THF (10 mL) was stirred under nitrogen and cooled in an ice-bath for 15 min. A solution of iso-propyl magnesium chloride (2M in THF, 8.1 mL) was added dropwise over 5 min and the reaction mixture was stirred for 1.5 h.

The above cold Grignard solution was added to a solution of (E)-N-(cyclopropylmethylene)-2-methylpropane-2-(R)-sulfinamide (2.55 g, 14.7 mmol) in DCM (50 mL) at −78° C. under nitrogen in four portions over 1 h. The reaction mixture was allowed to slowly warm to ambient temperature over 18 h. The reaction was quenched with saturated aqueous ammonium chloride (10 mL) and stirred for 1 h. The mixture was diluted with ethyl acetate (200 mL), washed with water, brine, dried with anhydrous sodium sulfate, filtered and evaporated to give 6.6 g of (R)-N-((1R)-(1-tert-butyl-5-(2,4,5-trifluorophenyl)-1H-pyrazol-4-yl)(cyclopropyl)methyl)-2-methylpropane-2-sulfinamide as a colorless oil. This material was used without further purification as described in Example 9.

### Example 11

### Preparation of (R)-1-tert-butyl-4-cyclopropyl-7,8-difluoro-5-(2-methoxy-4-(trifluoromethyl)phenylsulfonyl)-4,5-dihydro-1H-pyrazolo[4,3-c]quinoline

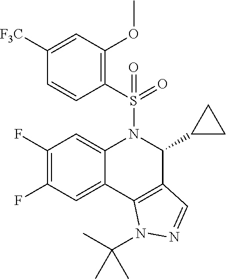

To a solution of N-((1R)-(5-(2-bromo-4,5-difluorophenyl)-1-tert-butyl-1H-pyrazol-4-yl)(cyclopropyl)methyl)-2-methoxy-4-(trifluoromethyl)benzenesulfonamide (820 mg, 1.32 mmol) in o-xylene (15 mL) under nitrogen was added copper(I) iodide (37.6 mg, 15 mol %) followed by N,N′-dimethylethylene diamine (87 mg, 75mol %). The mixture was stirred under nitrogen for several minutes, then anhydrous potassium carbonate (373 mg, 2.7 mmol, 2 eq) was added and flask was heated to between about 132 and 135° C. for 20 h. The mixture was cooled to ambient temperature and treated with saturated aqueous ammonium chloride (5 mL) for 2 h, diluted with ethyl acetate (100 mL) and water (50 mL). The organic layer was separated, washed with 1N HCl (50 mL), water (50 mL) and brine (50 mL), and was dried with sodium sulfate. The solvent was removed and the crude product was purified by flash chromatography (ethyl acetate/hexane; 1:2) to give the title compound as a colorless oil (614 mg, 86% yield). LC-MS m/z=486.1, 542.1, 564.1. ¹H-NMR (CDCl₃): 7.77 (m, 1H), 7.52 (m, 2H), 7.12 (s, 1H), 7.06 (m, 1H), 6.76 (m, 1H), 4.85 (m, 1H), 3.81 (s, 3H), 1.54 (s, 9H), 0.86 (m, 1H), 0.39 (m, 3H), 0.07 (m, 1H).

### Example 12

### Preparation of (R)-1-tert-butyl-4-cyclopropyl-7,8-difluoro-5-(3-methoxy-4-(trifluoromethyl)phenylsulfonyl)-4,5-dihydro-1H-pyrazolo[4,3-c]quinoline

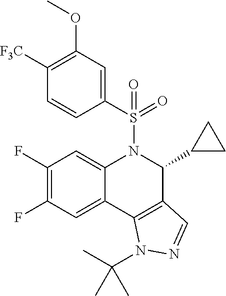

The title compound was synthesized from N-((1R)-(5-(2-bromo-4,5-difluorophenyl)-1-tert-butyl-1H-pyrazol-4-yl)(cyclopropyl)methyl)-3-methoxy-4-(trifluoromethyl)benzenesulfonamide (1500 mg, 2.41 mmol) according to the procedure outlined in Example 11, above (830 mg, 63% yield, colorless oil). LC-MS m/z=486.1, 542.1, 564.1. ¹H-NMR (CDCl₃): 7.82 (m, 1H), 7.50 (m, 1H), 7.33 (m, 1H), 7.24 (s, 1H), 6.90 (m, 1H), 6.74 (bs, 1H), 4.97 (m, 1H), 3.70 (s, 3H), 1.45 (s, 9H), 0.87 (m, 1H), 0.40 (m, 3H), 0.08 (m, 1H).

### Example 13

### Preparation of (R)-1-(t-butyl)-8-fluoro-4-deuterio-4-(1,2,2,3,3-pentadeuterio-cyclopropyl)-5-((6-(trifluoromethyppyridin-3-yl)sulfonyl)-4,5-dihydro-1H-pyrazolo[4,3-c]quinoline

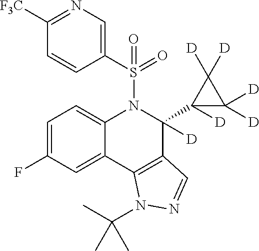

The title compound was synthesized from N-((1R)-(5-(2-bromo-5-fluorophenyl)-1-tert-butyl-1H-pyrazol-4-yl)deuterio(1,2,2,3,3-pentadeuteriocyclopropyl)methyl)-6-(trifluoromethyl)pyridine-3-sulfonamide (15 g, 25.8 mmol) according to the procedure outlined in Example 11, above, except that copper(I) iodide was used at 10mol %, and N,N′-dimethylethylene diamine was used at 20mol % (9.9 g, 77% yield, colorless solid). LC-MS m/z=445.1, 501.2, 523.2. ¹H-NMR (CDCl₃): 8.39 (bs, 1H), 7.91 (m, 1H), 7.75 (m, 1H), 7.46 (m, 2H), 7.20 (s, 1H), 7.15 (m, 1H), 1.47 (s, 9H).

### Example 14

### Preparation of (R)-1-tert-butyl-4-cyclopropyl-8-fluoro-5-(6-(trifluoromethyl)pyridin-3-ylsulfonyl)-4,5-dihydro-1H-pyrazolo[4,3-c]quinoline using a copper catalyst without an organic ligand

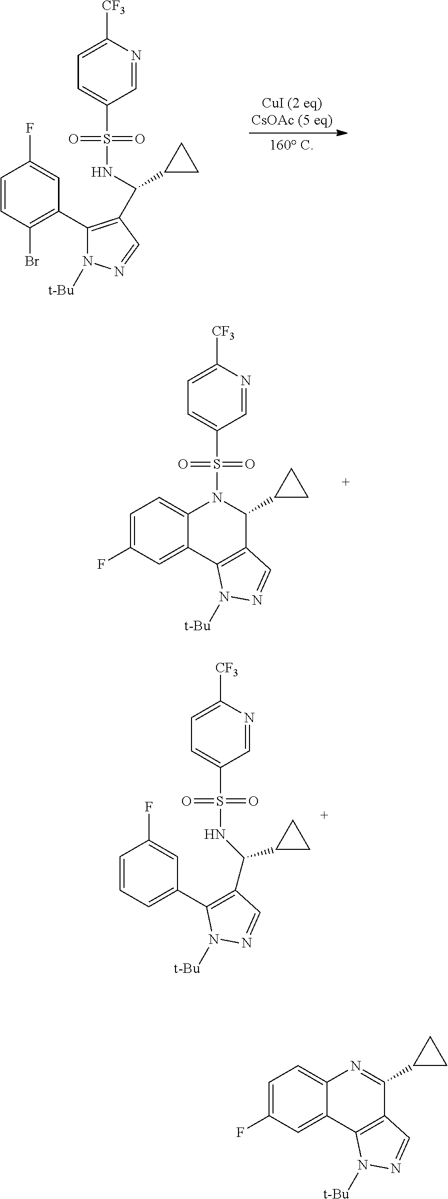

The title compound was prepared essentially as described in US Patent Application Publication No. US2008/0021056. To a slurry of copper iodide (2 equivalents) and cesium acetate (5 equivalents) in DMSO at 160° C. was rapidly added a solution of N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide in warm DMSO. The mixture was stirred for 10 minutes. It was then slowly cooled to ambient temperature and poured into saturated NaCl solution (brine). Water was added and the product was extracted with EtOAc. The combined organic phases were washed with water and brine, dried over Na₂SO₄, filtered and dried. The crude product was analyzed by LCMS and contained about 77.6% (AUC) of the cyclized title compound, 13% (AUC) of (R)-N-((1-tert-butyl-5-(3-fluorophenyl)-1H-pyrazol-4-yl)(cyclopropyl)methyl)-6-(trifluoromethyl)pyridine-3-sulfonamide) (de-brominated side-product) and 3.4% (AUC) of 1-tert-butyl-4-cyclopropyl-8-fluoro-1H-pyrazolo[4,3-c]quinoline (aromatized side product). The crude product was triturated with petroleum ether and reanalyzed by LCMS indicating about 89% (AUC) of the title compound and about 11% (AUC) de-brominated side product. Crude reaction mixtures generated using an improved method described in this disclosure (see, e.g., Example 4.4.) contain no detectable amounts of de-brominated side-product (e.g., less than 1% AUC), no detectable amounts of aromatized side-product (e.g., less than 1% AUC), and at least 80% (AUC) of the desired product.

What is claimed is:

1. A method of affecting an intra-molecular cyclization, the method comprising:
(i) contacting a first molecule having a structure according to Formula (I): 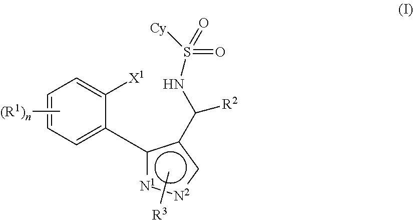
or a salt or solvate thereof,
wherein
n is an integer selected from 0 to 4;
N¹ and N² are nitrogen atoms of a pyrazole ring;
X¹ is F, Cl, Br, I, tosylate or mesylate;
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein each of the alkyl, alkenyl, alkynyl, heteroalkyl, cycloalkyl, heterocycloalkyl, aryl and heteroaryl is optionally substituted with from 1 to 3 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
R² is a member selected from H, alkyl, alkenyl, alkynyl, haloalkyl, cycloalkyl, heterocycloalkyl, aryl, heteroaryl, wherein each of the alkyl, alkenyl, alkynyl, haloalkyl, cycloalkyl, heterocycloalkyl, aryl, and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶;
R³ is an amino protecting group covalently bonded to N¹ or N² of the pyrazole; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
with a catalyst comprising copper and at least one organic ligand, under reaction conditions sufficient to form a second molecule having a structure according to Formula (II): 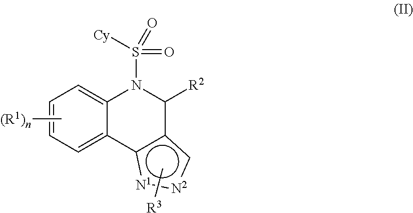
or a salt or solvate thereof, wherein Cy, n, R¹, R² and R³ are defined as for Formula (I).

2. The method of claim 1, wherein R³ is covalently bound to N¹ of the pyrazole ring.

3. The method of claim 1, wherein the contacting occurs in the presence of a base.

4. The method of claim 3, wherein the base is a member selected from carbonate, phosphate and acetate.

5. The method of claim 1, wherein the organic ligand is a member selected from 1,2-diamines and N,N-dialkylsalicylamides.

6. The method of claim 1, wherein the organic ligand is a member selected from N¹,N²-dialkylcyclohexane-1,2-diamines and N¹,N²-dialkylethane-1,2-diamines.

7. The method of claim 1, wherein the organic ligand is N,N′-dimethylethylenediamine (DMEDA) or N,N-diethylsalicylamide (DESA).

8. The method of claim 1, wherein the copper is present in an amount equivalent to between about 0.1 mol % and about 10 mol % relative to the first molecule.

9. The method of claim 1, wherein the copper is present in an amount equivalent to between about 0.5 mol % and about 5 mol % relative to the first molecule.

10. The method of claim 1, wherein the copper is present in an amount equivalent to between about 1 mol % and about 3 mol % relative to the first molecule.

11. The method of claim 1, wherein the organic ligand is present in an amount equivalent to between about 1 mol % and about 20 mol % relative to the first molecule.

12. The method of claim 1, wherein the organic ligand is present in an amount equivalent to between about 5 mol % and about 15 mol % relative to the first molecule.

13. The method of claim 1, wherein the second molecule is formed with a reaction yield between about 80% and about 100% (mol/mol) relative to the first molecule.

14. The method of claim 1, wherein the contacting occurs in the presence of a solvent selected from xylene and toluene.

15. The method of claim 1, wherein the reaction conditions comprise heating to between about 100° C. and about 150° C.

16. The method of claim 1, wherein the reaction conditions comprise heating to between about 100° C. and about 150° C. for a period between about 2 h and about 72 h.

17. The method of claim 1, wherein R³ is selected from (C₁-C₆)alkyl and benzyl.

18. The method of claim 17, wherein R³ is tert-butyl.

19. The method of claim 1, wherein X¹ is Br.

20. The method of claim 1, wherein X¹ is F.

21. The method of claim 1, wherein n is 1 or 2 and each R¹ is halogen.

22. The method of claim 1, wherein Cy is selected from optionally substituted phenyl and optionally substituted pyridinyl.

23. The method of claim 22, wherein Cy is CF₃-substituted phenyl or CF₃-substituted pyridinyl.

24. The method of claims 1, wherein R² is (C₁-C₃)cycloalkyl.

25. The method of claims 24, wherein R² is cyclopropyl.

26. The method of claim 1, wherein the first molecule has a structure according to Formula (Ie): 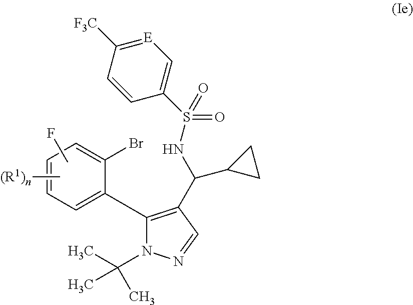
or a salt or solvate thereof,
wherein
p is 0 or 1; and
E is N or CH.

27. The method of claim 1, further comprising purifying the second molecule using a method comprising:
(a) heating the second molecule in a mixture comprising methanol and water, thereby forming a solution;
(b) cooling the solution of step (a), thereby forming a precipitate of the second molecule; and
(c) isolating the precipitate of step (b).

28. The method of claim 27, wherein the mixture of step (a) comprises water in an amount equivalent to between about 5% (v/v) and about 15% (v/v).

29. The method of claim 28, wherein the mixture of step (a) comprises water in an amount equivalent to between about 8% (v/v) and about 12% (v/v).

30. The method of claim 1 further comprising:
(ii) removing the amino protecting group R³ from the second molecule, thereby forming a third molecule having a structure according to Formula (A): 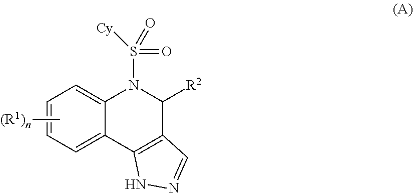
or a salt or solvate thereof, wherein Cy, n, R¹ and R² are defined as for Formula (I) in claim 1.

31. The method of claim 30, wherein the removing is accomplished using aqueous formic acid and heat, thereby forming an acidic reaction mixture.

32. The method of claim 31, further comprising isolating the third molecule of Formula (A) by:
(a) mixing the acidic reaction mixture with a sufficient amount of water, thereby forming a precipitate; and
(b) isolating the precipitate.

33. The method of claim 31, further comprising: purifying the third molecule using a method comprising:
(a) forming a solution of the third molecule in ethanol;
(b) adding the solution of step (a) to water, thereby forming a precipitate of the third molecule; and
(c) isolating the precipitate.

34. A method comprising:
(ii) contacting a first compound having a structure according to Formula (X): 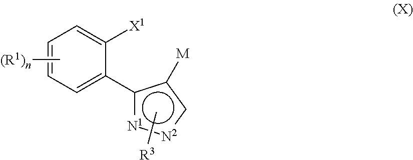
wherein
M is selected from Li and MgX, wherein X is halogen;
n is an integer selected from 0 to 4;
N¹ and N² are nitrogen atoms of a pyrazole ring;
X¹ is F, Cl, Br, I, tosylate or mesylate;
R³ is an amino protecting group covalently bonded to N¹ or N²; and
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein
each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
with a sulfinylimine having a structure according to Formula (XI): 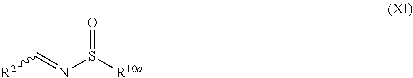
wherein
R² is selected from H, alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶; and
R^{10a} is selected from alkyl, alkenyl, alkynyl, haloalkyl, cycloalkyl, heterocycloalkyl, aryl and heteroaryl, each optionally substituted with from 1 to 5 substituents selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
thereby forming a second compound having a structure according to Formula (XII): 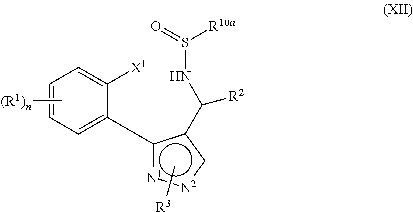
or a salt or solvate thereof.

35. The method of claim 34, wherein R³ is covalently bonded to N¹ of the pyrazole.

36. The method of claim 34, wherein X¹ is Br.

37. The method of claim 34, wherein X¹ is F.

38. The method of claim 34, wherein M is MgX and X is Cl or Br.

39. The method of claim 34, wherein n is 1 or 2 and each R¹ is F.

40. The method of claim 34, wherein the first compound has a structure according to Formula (Xh) or (Xi): 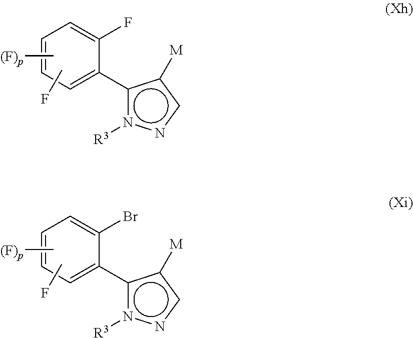
or a salt or solvate thereof, wherein p is an integer selected from 1, 2 and 3.

41. The method of claims 34, further comprising:
(ii) removing a sulfinyl moiety from the second compound of Formula (XII), thereby forming a third compound having a structure according to Formula (XIII): 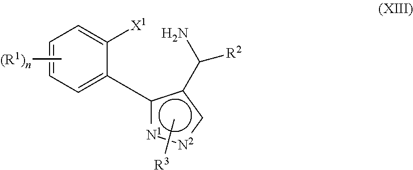
or a salt or solvate thereof, wherein N¹, N², X¹, n, R¹, R² and R³ are defined as in claim 34.

42. The method of claim 41, wherein the removing of the sulfinyl moiety is accomplished using one or more acids.

43. The method of claim 42, wherein the acid is HCl.

44. The method of claim 41, further comprising:
(iii) contacting the third compound of Formula (XIII) with a sulfonylchloride having the formula:
Cy-S(O)₂Cl
wherein
Cy is a member selected from aryl, heteroaryl, cycloalkyl, and heterocycloalkyl, each optionally substituted with from 1 to 5 substituents selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
thereby forming a fourth compound having a structure according to Formula (I): 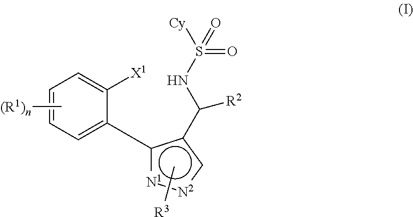
or a salt or solvate thereof, wherein X¹, n, R¹, R² and R³ are defined as in claim 34.

45. The method of claim 44, further comprising cyclizing the compound of Formula (I) to form a compound of formula II

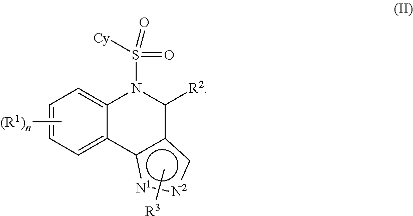

46. The method of claim 45, further comprising removing the amino protecting group R³.

47. A method comprising:
(j) contacting a first compound having a structure according to Formula (Xm): 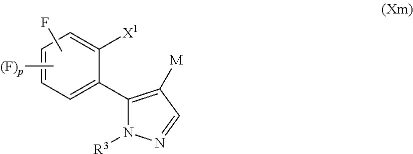
or a salt or solvate thereof,
wherein
M is Li or MgX, wherein X is Cl, Br or I;
X¹ is F, Cl or Br;
p is 0 or 1; and
R³ is an amino protecting group,
with a sulfinylimine having a structure according to Formula (XIa): 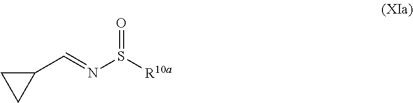
wherein R^{10a} is branched (C₃-C₈)alkyl, branched 3- to 8-membered heteroalkyl, (C₃-C₁₀)cycloalkyl, 3- to 6-membered heterocycloalkyl, aryl, and 5- or 6-membered heteroaryl,
under reaction conditions sufficient to form a second compound having a structure according to Formula (XIIa): 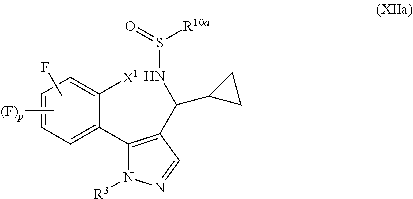
or a salt or solvate thereof; and
(ii) removing a sulfinyl moiety from the second compound of Formula (XIIa), thereby forming a third compound having a structure according to Formula (XIIIa): 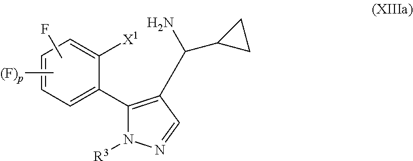
or a salt or solvate thereof.

48. The method of claim 47, wherein the removing of step (ii) is accomplished using acid.

49. The method of claim 48, wherein the acid is HCl.

50. The method of any of claim 47, wherein the first compound has a structure selected from:

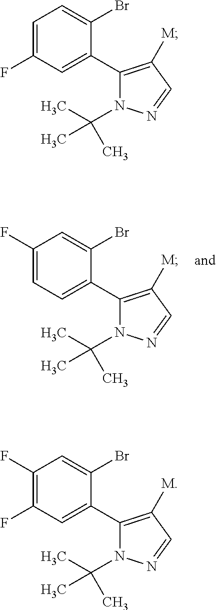

51. The method of claim 47 further comprising:
(iii) contacting the third compound of Formula (XIIIa) with a sulfonylchloride having the formula: 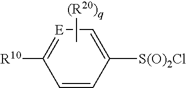
wherein
E is CH or N;
q is an integer selected from 0 and 1;
R¹⁰ is selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR²⁴, SR²⁴, NR²⁴R²⁵, C(O)R²⁶, C(O)NR²⁴R²⁵, OC(O)NR²⁴R²⁵, C(O)OR²⁴, NR²⁷C(O)R²⁶, NR²⁷C(O)OR²⁴, NR²⁷C(O)NR²⁴R²⁵, NR²⁷C(S)NR²⁴R²⁵, NR²⁷S(O)₂R²⁶, S(O)₂NR²⁴R²⁵, S(O)R²⁶ and S(O)₂R²⁶,
wherein
R²⁴, R²⁵ and R²⁷ are independently selected from H, acyl, C₁-C₆-alkyl, C₁-C₆ haloalkyl, 2- to 6-membered heteroalkyl, aryl, 5- or 6-membered heteroaryl, C₃-C₈ cycloalkyl and 3- to 8-membered heterocycloalkyl, wherein R²⁴ and R²⁵, together with the nitrogen atom to which they are bound are optionally joined to form a 5- to 7-membered heterocyclic ring; and
R²⁶ is independently selected from acyl, C₁-C₆-alkyl, C₁-C₆ haloalkyl, 2- to 6-membered heteroalkyl, aryl, 5- or 6-membered heteroaryl, C₃-C₈ cycloalkyl and 3- to 8-membered heterocycloalkyl; and
R²⁰ is selected from OH and (C₁-C₃)alkoxy,
under reaction conditions sufficient to form a fourth compound having a structure according to Formula (C): 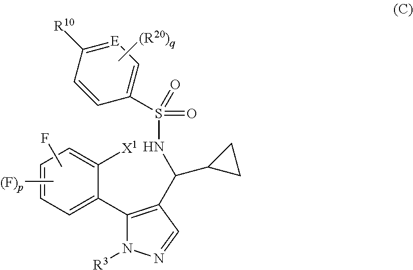
or a salt or solvate thereof; and
(iv) contacting the fourth compound of Formula (C) with a catalyst comprising copper and at least one organic ligand selected from N,N-dialkylsalicylamides, N¹,N²-dialkylcyclohexane-1,2-diamines and N¹,N²-dialkylethane-1,2-diamines,
under reaction conditions sufficient to form a fifth compound having a structure according to Formula (D): 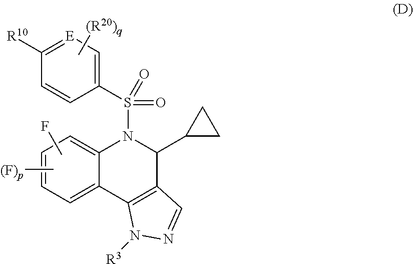
or a salt or solvate thereof.

52. The method of claim 47 or 51, wherein R³ is selected from (C₁-C₆)alkyl and benzyl.

53. The method of claim 47 or 51, wherein R³ is tert-butyl.

54. The method of claim 47 or 51, wherein X¹ is Br.

55. The method of claim 51, wherein R¹⁰ is selected from C₁-C₃-alkyl, C₁-C₃-haloalkyl, CN and halogen.

56. The method of claim 51, wherein R¹⁰ is CF₃.

57. The method of claim 51, wherein the contacting of step (iv) occurs in the presence of a base.

58. The method of claim 57, wherein the base is a member selected from carbonate, phosphate and acetate.

59. The method of claims 51, wherein the organic ligand is N,N′-dimethylethylenediamine (DMEDA) or N,N-diethylsalicylamide (DESA).

60. The method of claim 51, wherein the copper is present in an amount equivalent to between about 1 mol % and about 10 mol % relative to the first molecule.

61. The method of claim 51, wherein the copper is present in an amount equivalent to between about 1 mol % and about 5 mol % relative to the first molecule.

62. The method of claim 51, wherein the organic ligand is present in an amount equivalent to between about 1 mol % and about 20 mol % relative to the first molecule.

63. The method of claim 51, wherein the organic ligand is present in an amount equivalent to between about 5 mol % and about 15 mol % relative to the first molecule.

64. The method of claim 51, wherein the copper is present in an amount equivalent to between about 1 mol % and about 3 mol % relative to the first molecule, and the organic ligand is present in an amount equivalent to between about 8 mol % and about 12 mol % relative to the first molecule.

65. The method of claim 51, wherein the contacting of step (iv) occurs in the presence of a solvent selected from xylene and toluene.

66. The method of claim 51, wherein the reaction conditions of step (iv) comprise heating to between about 100° C. and about 150° C. for a period between about 2 h and about 72 h.

67. The method of claim 51, wherein the fourth compound has a structure selected from: 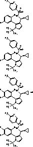
or a salt or solvate thereof.

68. The method of claim 51, wherein the second compound of Formula (XIIa), the third compound of Formula (XIIIa), and the fourth compound of Formula (C) are not isolated prior to subsequent reaction steps.

69. The method of claim 51 further comprising: purifying the fifth compound using a method comprising:
(a) heating the fifth compound in a mixture comprising water in an amount equivalent to between about 5% (v/v) and about 15% (v/v) and methanol, thereby forming a solution;
(b) cooling the solution of step (a), thereby forming a precipitate of the fifth compound; and
(c) isolating the precipitate of step (b).

70. The method of claim 69, wherein the fifth compound is isolated with an overall reaction yield of at least about 50% (mol/mol) relative to the first compound of Formula (I).

71. The method of claim 51 further comprising:
(v) removing the amino protecting group from the fifth molecule, thereby forming a reaction mixture comprising a sixth compound having a structure according to Formula (E): 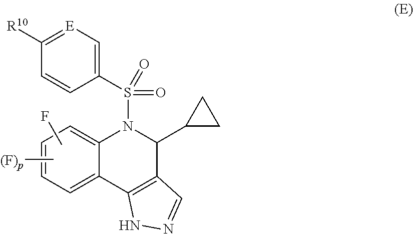
or a tautomer or mixture of tautomers thereof.

72. The method of claim 71, wherein the removing of step (v) is accomplished using aqueous formic acid and heat.

73. The method of claim 72, further comprising: isolating the sixth compound by
(a) mixing the reaction mixture of step (v) with a sufficient amount of water, thereby forming a precipitate; and
(b) isolating the precipitate.

74. The method of claim 71, further comprising: purifying the sixth compound using a method comprising:
(a) forming a solution of the sixth compound in a solvent comprising ethanol;
(b) contacting the solution of step (a) with a sufficient amount of water to form a precipitate of the sixth compound; and
(c) isolating the precipitate.

75. A method of affecting an intra-molecular cyclization, the method comprising:
(i) contacting a first molecule having a structure according to Formula (III): 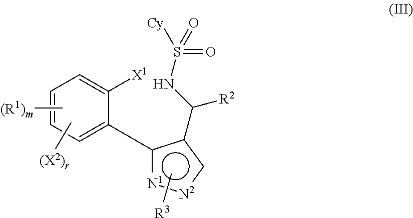
or a salt or solvate thereof,
wherein
r is an integer selected from 2 to 4;
m is an integer selected from 0 to 2, provided that the sum of m and r is not greater than 4;
N¹ and N² are nitrogen atoms of a pyrazole ring;
X¹ is F, Cl, Br, I, tosylate or mesylate;
X² is F, Cl or Br;
R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein each of the alkyl, alkenyl, alkynyl, heteroalkyl, cycloalkyl, heterocycloalkyl, aryl and heteroaryl is optionally substituted with from 1 to 3 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
R² is a member selected from H, alkyl, alkenyl, alkynyl, haloalkyl, cycloalkyl, heterocycloalkyl, aryl, heteroaryl, wherein each of the alkyl, alkenyl, alkynyl, haloalkyl, cycloalkyl, heterocycloalkyl, aryl, and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶, and S(O)₂R¹⁶;
R³ is an amino protecting group covalently bonded to N¹ or N² of the pyrazole; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl,
with a base, in the absence of a metal catalyst, under reaction conditions sufficient to form a second molecule having a structure according to Formula (IV): 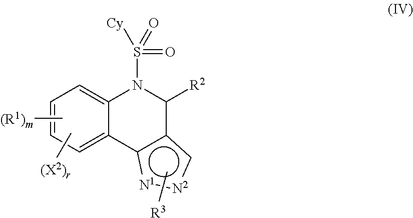
or a salt or solvate thereof, wherein Cy, m, r, X², R¹, R² and R³ are defined as for Formula (I).

76. The method of claim 75, wherein the base of step (i) is selected from potassium carbonate, sodium carbonate and cesium carbonate.

77. The method of claim 75, wherein X¹ is F.

78. The method of claim 77, wherein r is 2 and each X² is F.

79. The method of claim 77, wherein R³ is covalently bonded to N¹ of the pyrazole ring.

80. A compound having a structure according to Formula (XX): 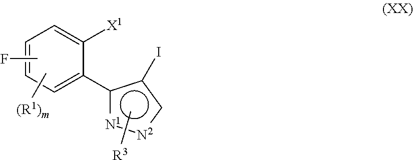
or a salt or solvate thereof,
wherein
N¹ and N² are nitrogen atoms of a pyrazole ring;
I is iodine;
X¹ is halogen;
R³ is an amino protecting group covalently bonded to N¹ or N² of the pyrazole ring;
m is an integer selected from 0 to 3; and
each R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
R¹⁴, R¹⁵, and R¹⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl;
R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.

81. The compound of claim 80, wherein R³ is covalently bonded to N¹ of the pyrazole ring.

82. The compound of claim 80, wherein R^{3a} is (C₁-C₆)alkyl or benzyl.

83. The compound of claim 80, wherein X¹ is Br.

84. The compound of claim 80, wherein X¹ is F.

85. The compound of claim 80, wherein m is 0.

86. The compound of claim 80, wherein m is 1 or 2 and each R¹ is halogen.

87. The compound of claim 80 having a structure according to Formula (XXIc) or Formula (XXId): 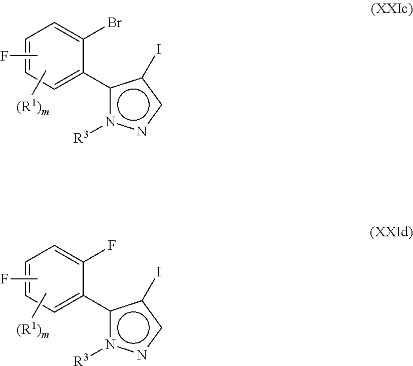
wherein m, R¹ and R³ are defined as for Formula (XX) in claim 75.

88. The compound of claim 80 having a structure selected from: 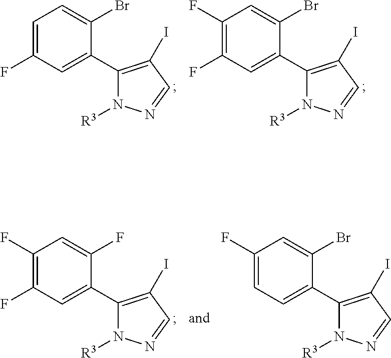
or a salt or solvate thereof.

89. A compound having a structure according to Formula (XXII): 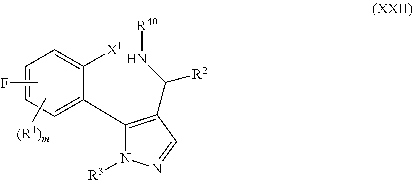
or a salt or solvate thereof,
wherein
X¹ is halogen;
R³ is an amino protecting group;
m is an integer selected from 0 to 3;
each R¹ is a member independently selected from alkyl, alkenyl, alkynyl, haloalkyl, heteroalkyl, cycloalkyl, heterocycloalkyl, aryl, heteroaryl, CN, halogen, OR⁴, SR⁴, NR⁴R⁵, C(O)R⁶, C(O)NR⁴R⁵, OC(O)NR⁴R⁵, C(O)OR⁴, NR⁷C(O)R⁶, NR⁷C(O)OR⁴, NR⁷C(O)NR⁴R⁵, NR⁷C(S)NR⁴R⁵, NR⁷S(O)₂R⁶, S(O)₂NR⁴R⁵, S(O)R⁶ and S(O)₂R⁶,
wherein each of the alkyl, alkenyl, alkynyl, heteroalkyl, cycloalkyl, heterocycloalkyl, aryl and heteroaryl is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
R⁴, R⁵, and R⁷ are independently selected from H, acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R⁴ and R⁵, together with the nitrogen atom to which they are bound, are optionally joined to form a 5- to 7-membered heterocyclic ring; and
R⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.
R² is selected from H, alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶, with the proviso that R² is other than carboxyl or carboxyl-substituted C₁-C₃-alkyl;
R⁴⁰ is selected from H, alkyl, alkenyl, alkynyl, haloalkyl, cycloalkyl, heterocycloalkyl, aryl, heteroaryl, S(O)R^{10a}, and S(O)₂Cy,
wherein
each of the alkyl, alkenyl, alkynyl, haloalkyl, cycloalkyl, heterocycloalkyl, aryl, and heteroaryl of R⁴⁰ is optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶;
R^{10a} is selected from alkyl, alkenyl, alkynyl, haloalkyl, cycloalkyl, heterocycloalkyl, aryl, heteroaryl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶; and
Cy is a member selected from aryl, heteroaryl, cycloalkyl, and heterocycloalkyl, each optionally substituted with from 1 to 5 substituents independently selected from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR¹⁴, SR¹⁴, NR¹⁴R¹⁵, C(O)R¹⁶, C(O)NR¹⁴R¹⁵, OC(O)NR¹⁴R¹⁵, C(O)OR¹⁴, NR¹⁷C(O)R¹⁶, NR¹⁷C(O)OR¹⁴, NR¹⁷C(O)NR¹⁴R¹⁵, NR¹⁷C(S)NR¹⁴R¹⁵, NR¹⁷S(O)₂R¹⁶, S(O)₂NR¹⁴R¹⁵, S(O)R¹⁶ and S(O)₂R¹⁶,
wherein
each R¹⁴, each R¹⁵, and each R¹⁷ is independently selected from H, acyl, C₁-C₆-alkyl, C₁-C₆ haloalkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl, wherein R¹⁴ and R¹⁵, together with the nitrogen atom to which they are bound, are optionally joined to form a 5- to 7-membered heterocyclic ring; and
each R¹⁶ is selected from acyl, C₁-C₆-alkyl, C₁-C₆-alkenyl, C₁-C₆-alkynyl, 2- to 6-membered heteroalkyl, aryl, 5- or 6-membered heteroaryl, C₃-C₈ cycloalkyl and 3- to 8-membered heterocycloalkyl.

90. The compound of claim 89, wherein R⁴⁰ is selected from H, S(O)R^{10a}, and S(O)₂Cy.

91. The compound of claim 89, wherein R⁴⁰ is H.

92. The compound of claim 89, wherein R⁴⁰ is S(O)R^{10a}wherein e^{a} is branched (C₃-C₈)alkyl, branched 3- to 8-membered heteroalkyl, (C₃-C₁ₒ)cycloalkyl, 3- to 6-membered heterocycloalkyl, aryl, and 5- or 6-membered heteroaryl.

93. The compound of claim 89, wherein R⁴⁰ is S(O)₂Cy, wherein Cy is selected from aryl, and 5- or 6-membered heteroaryl, wherein the aryl or heteroaryl is optionally substituted with from 1 to 3 substituents selected from C₁-C₃-alkyl, C₁-C₃-alkenyl, C₁-C₃-alkynyl, C₁-C₃-haloalkyl, halogen, CN, OH and methoxy

94. A compound selected from:
5-(2-bromo-5-fluorophenyl)-1-tert-butyl-4-iodo-1H-pyrazole;
5-(2-bromo-4-fluorophenyl)-1-tert-butyl-4-iodo-1H-pyrazole;
5-(2-bromo-4,5-difluorophenyl)-1-tert-butyl-4-iodo-1H-pyrazole; and
1-tert-butyl-4-iodo-5-(2,4,5-trifluorophenyl)-1H-pyrazole;
(5-(2-bromo-5-fluorophenyl)-1-tert-butyl-1H-pyrazol-4-yl)(cyclopropyl)methanamine;
(5-(2-bromo-4-fluorophenyl)-1-tert-butyl-1H-pyrazol-4-yl)(cyclopropyl)methanamine;
(5-(2-bromo-4,5-difluorophenyl)-1-tert-butyl-1H-pyrazol-4-yl)(cyclopropyl)-methanamine;
(1-tert-butyl-5-(2,4,5-trifluorophenyl)-1H-pyrazol-4-yl)(cyclopropyl)methanamine;
(1R)- (5-(2-bromo-5-fluorophenyl)-1-tert-butyl-1H-pyrazol-4-yl)(cyclopropyl)methanamine;
(1R)- (5-(2-bromo-4-fluorophenyl)-1-tert-butyl-1H-pyrazol-4-yl)(cyclopropyl)methanamine;
(1R)-(5-(2-bromo-4,5-difluorophenyl)-1-tert-butyl-1H-pyrazol-4-yl)(cyclopropyl)methanamine; and
(1R)-(1-tert-butyl-5-(2,4,5-trifluorophenyl)-1H-pyrazol-4-yl)(cyclopropyl)methanamine;
N-((5-(2-bromo-5-fluorophenyl)-1-tert-butyl-1H-pyrazol-4-yl)(cyclopropyl)methyl)-2-methylpropane-2-sulfinamide;
N-((1R)-(5-(2-bromo-5-fluorophenyl)-1-tert-butyl-1H-pyrazol-4-yl)(cyclopropyl)methyl)-2-methylpropane-2-sulfinamide;
N-((5-(2-bromo-4,5-difluorophenyl)-1-tert-butyl-1H-pyrazol-4-yl)(cyclopropyl)methyl)-2-methylpropane-2-sulfinamide;
N-((1R)-(5-(2-bromo-4,5-difluorophenyl)-1-tert-butyl-1H-pyrazol-4-yl)(cyclopropyl)methyl)-2-methylpropane-2-sulfinamide;
N-((5-(2-bromo-4-fluorophenyl)-1-tert-butyl-1H-pyrazol-4-yl)(cyclopropyl)methyl)-2-methylpropane-2-sulfinamide;
N-((1R)-(5-(2-bromo-4-fluorophenyl)-1-tert-butyl-1H-pyrazol-4-yl)(cyclopropyl)methyl)-2-methylpropane-2-sulfinamide;
N-((1-tert-butyl-5-(2,4,5-trifluorophenyl)-1H-pyrazol-4-yl)(cyclopropyl)methyl)-2-methylpropane-2-sulfinamide; and
N-((1R)-(1-tert-butyl-5-(2,4,5-trifluorophenyl)-1H-pyrazol-4-yl)(cyclopropyl)methyl)-2-methylpropane-2-sulfinamide;
N-((5-(2-bromo-4-fluorophenyl)-1-tert-butyl-1H-pyrazol-4-yl)(cyclopropyl)methyl)-4-(trifluoromethyl)benzenesulfonamide;
N-((1R)-(5-(2-bromo-4-fluorophenyl)-1-tert-butyl-1H-pyrazol-4-yl)(cyclopropyl)-methyl)-4-(trifluoromethyl)benzenesulfonamide;
N-((5-(2-bromo-4-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4-fluorophenyl)-1-tert-butyl-1H-pyrazol-4-yl)(cyclopropyl)methyl)-6-(trifluoromethyl)pyridine-3-sulfonamide;
N-((5-(2-bromo-4,5-difluorophenyl)-1-tert-butyl-1H-pyrazol-4-yl)(cyclopropyl)methyl)-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-4-(trifluoromethyl)benzenesulfonamide;
N-((5-(2-bromo-4,5-difluorophenyl)-1-tert-butyl-1H-pyrazol-4-yl)(cyclopropyl)methyl)-6-(trifluoromethyl)pyridine-3-sulfonamide;
N-((1R)-(5-(2-bromo-4,5-difluorophenyl)-1-tert-butyl-1H-pyrazol-4-yl)(cyclopropyl)methyl)-6-(trifluoromethyl)pyridine-3-sulfonamide;
N-((5-(2-bromo-5-fluorophenyl)-1-tert-butyl-1H-pyrazol-4-yl)(cyclopropyl)methyl)-4-(trifluoromethyl)benzenesulfonamide;
N-((1R)-(5-(2-bromo-5-fluorophenyl)-1-tert-butyl-1H-pyrazol-4-yl)(cyclopropyl)methyl)-4-(trifluoromethyl)benzenesulfonamide;
N-((5-(2-bromo-5-fluorophenyl)-1-tert-butyl-1H-pyrazol-4-yl)(cyclopropyl)methyl)-6-(trifluoromethyl)pyridine-3-sulfonamide;
N-((1R)-(5-(2-bromo-5-fluorophenyl)-1-tert-butyl-1H-pyrazol-4-yl)(cyclopropyl)methyl)-6-(trifluoromethyl)pyridine-3-sulfonamide;
N-((1-tert-butyl-5-(2,4,5-trifluorophenyl)-1H-pyrazol-4-yl)(cyclopropyl)methyl)-4-(trifluoromethyl)benzenesulfonamide;
N-((1R)-(1-tert-butyl-5-(2,4,5-trifluorophenyl)-1H-pyrazol-4-yl)(cyclopropyl)methyl)-4-(trifluoromethyl)benzenesulfonamide;
N-((1-tert-butyl-5-(2,4,5-trifluorophenyl)-1H-pyrazol-4-yl)(cyclopropyl)methyl)-6-(trifluoromethyl)pyridine-3-sulfonamide;
N-((1R)-(1-tert-butyl-5-(2,4,5-trifluorophenyl)-1H-pyrazol-4-yl)(cyclopropyl)methyl)-6-(trifluoromethyl)pyridine-3-sulfonamide,
N-((1R)-(1-tert-butyl-5-(2,4,5-trifluorophenyl)-1H-pyrazol-4-yl)(cyclopropyl)methyl)-3-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-5-fluorophenyl)-1-tert-butyl-1H-pyrazol-4-yl)(cyclopropyl)methyl)-3-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-3-methoxy-4-(trifluoromethyl)benzenesulfonamide;
N-((1R)-(1-tert-butyl-5-(2,4,5-trifluorophenyl)-1H-pyrazol-4-yl)(cyclopropyl)methyl)-2-methoxy-4-(trifluoromethyl)benzenesulfonamide;
N-((1R)-(5-(2-bromo-4,5-difluorophenyl)-1-tert-butyl-1H-pyrazol-4-yl)(cyclopropyl)methyl)-2-methoxy-4-(trifluoromethyl)benzenesulfonamide;
N-((1R)-(5-(2-bromo-4-fluorophenyl)-1-tert-butyl-1H-pyrazol-4-yl)(cyclopropyl)methyl)-2-methoxy-4-(trifluoromethyl)benzenesulfonamide; and
N-((1R)-(5-(2-bromo-5-fluorophenyl)-1-tert-butyl-1H-pyrazol-4-yl)(cyclopropyl)methyl)-2-methoxy-4-(trifluoromethyl)benzenesulfonamide,
or a salt, solvate, tautomer or mixture of tautomers thereof.
